SEQUENCE LISTING (1 sequence)

SEQ ID NO: 1 - protein, 21 aa, Mouse
RLGWWRFYTLRRARQGNTKQR

(19) United States
(12) Patent Application Publication
(10) Pub. No.: US 2014/0288043 A1
(43) Pub. Date: Sep. 25, 2014

(54) PYRAZOLOPYRIDINE COMPOUNDS

(71) Applicant: Genentech, Inc., South San Francisco, CA (US)

(72) Inventors: Bryan Chan, San Carlos, CA (US); Anthony Estrada, San Carlos, CA (US); Daniel Shore, San Francisco, CA (US); Zachary Sweeney, Redwood City, CA (US)

(73) Assignee: Genentech, Inc., South San Francisco, CA (US)

(21) Appl. No.: 13/847,409

(22) Filed: Mar. 19, 2013

(51) Int. Cl. C07D 471/04; C07D 519/00

(52) U.S. Cl. CPC C07D 471/04; C07D 519/00
USPC 514/210.18; 514/234.2; 514/256; 514/269; 514/303; 544/122; 544/127; 544/319; 544/333; 546/119

(57) ABSTRACT

A first aspect of the invention relates to a compound of formula (1A) or (1B), or a pharmaceutically acceptable salt thereof,

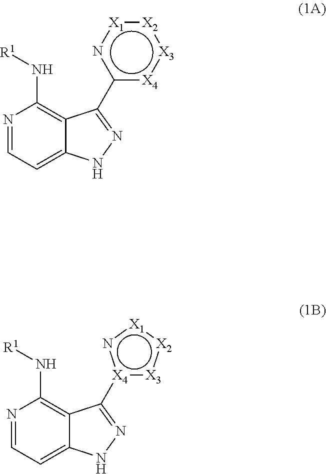

Wherein R¹ is a group selected from alkyl, monocyclic heterocycloalkyl, bicyclic heterocycloalkyl and cycloalkyl, each of which is optionally substituted, and wherein X₁, X₂, X₃ and X₄ are as defined herein.
   Further aspects relate to pharmaceutical compositions, therapeutic uses and process for preparing compounds of formula (1A) and (1B).

The present invention relates to pyrazolopyridine compounds that are capable of inhibiting one or more kinases, more particularly, LRRK2. The compounds find applications in the treatment of a variety of disorders, including cancer and neurodegenerative diseases such as Parkinson's disease.

### BACKGROUND TO THE INVENTION

There has been much interest raised by the recent discovery that different autosomal dominant point mutations within the gene encoding for LRRK2 predispose humans to develop late-onset PD (OMIM accession number 609007), with a clinical appearance indistinguishable from idiopathic PD [1-3]. The genetic analysis undertaken to date indicates that mutations in LRRK2 are relatively frequent, not only accounting for 5-10% of familial PD, but also being found in a significant proportion of sporadic PD cases [4, 5]. Little is known about how LRRK2 is regulated in cells, what its physiological substrates are and how mutations cause or increase risk of PD.

The domain structure of LRRK2 is shown in FIG. 1, which also depicts the mutations that have thus far been reported in patients with PD. The defining feature of the LRRK2 enzyme is a Leucine Rich Repeat (LRR) motif (residues 1010-1291), a Ras-like small GTPase (residues 1336-1510), a region of high amino acid conservation that has been termed the C-terminal Of Ras of complex (COR) domain (residues 1511-1878), a protein kinase catalytic domain (residues 1879-2132) and a C-terminal WD40 motif (2231-2276) [6, 7]. The protein kinase domain of LRRK2 belongs to the tyrosine-like serine/threonine protein kinases and is most similar to the kinase RIP (Receptor Interacting Protein), which play key roles in innate immunity signalling pathways [8]. To date, almost 40 single amino acid substitution mutations have been linked to autosomal-dominant PD and the location of these mutations is illustrated in FIG. 1A ([2, 3]). The most prevalent mutant form of LRRK2 accounting for approximately 6% of familial PD and 3% of sporadic PD cases in Europe, comprises an amino acid substitution of Gly2019 to a Ser residue. Gly2019 is located within the conserved DYG-Mg²⁺-binding motif, in subdomain-VII of the kinase domain [2]. Recent reports suggest that this mutation enhances the autophosphorylation of LRRK2, as well as its ability to phosphorylate myelin basic protein 2-3-fold [9, 10], a finding confirmed by the Applicant [11]. These observations suggest that over-activation of LRRK2 predisposes humans to develop PD, implying that drugs which inhibited LRRK2, could be utilised to halt progression or even perhaps reverse symptoms of some forms of PD.

The study of LRRK2 has been hampered by the difficulty in expressing active recombinant enzyme and by the lack of a robust quantitative assay. In work undertaken by the Applicant, an active recombinant fragment of LRRK2 containing the GTPase-COR and kinase domains encompassing residues 1326-2527 was expressed in 293 cells [11]. The more active G2019S mutant of this LRRK2 fragment was utilised in a KinasE Substrate TRacking and ELucidation (KESTREL) screen in an initial attempt to identify physiological substrates (reviewed in [14]). This led to the identification of a protein termed moesin, which was efficiently phosphorylated by LRRK2 in vitro [11]. Moesin is a member of the Ezrin/Radixin/Moesin (ERM) family of proteins which functions to anchor the actin cytoskeleton to the plasma membrane and plays an important role in regulating membrane structure and organization [15, 16]. It was found that LRRK2 phosphorylated moesin at Thr558 [11], a previously characterised physiologically relevant phosphorylation site [15, 16]. LRRK2 also phosphorylated ezrin and radixin at the equivalent Thr residue. Phosphorylation of ERM proteins at the residue equivalent to Thr558, opens up the structures of these proteins and enables them to interact with actin microfilaments at their C-terminal residues and phosphoinositides and plasma membrane proteins through an N-terminal FERM domain. These findings were utilised to develop a robust and quantitative assay for LRRK2, based upon the phosphorylation of moesin or a short peptide that encompasses the Thr558 residue of moesin which is also efficiently phosphorylated by LRRK2 [11]. These assays were further adapted to develop an improved assay based on the use of the Nictide peptide [17].

Small molecule inhibitors of LRRK are described in WO 2010/106333 and WO 2011/141756, both in the name of Medical Research Council Technology.

The present invention seeks to provide further compounds that are capable of inhibiting one or more kinases, more particularly LRRK2.

### SUMMARY OF THE INVENTION

A first aspect of the invention relates to a compound of formula (IA) or (IB), or a pharmaceutically acceptable salt thereof,

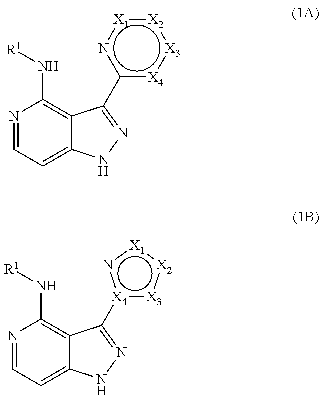

wherein:

R¹ is a group selected from alkyl, monocyclic heterocycloalkyl, bicyclic heterocycloalkyl and cycloalkyl, each of which is optionally substituted by one or more groups selected from alkyl, halo and cycloalkyl;

where for formula (1A):
one, two or three of X₁, X₂, X₃ and X₄ are N, and the remainder are each independently CR²; or
X₁, X₂, X₃ and X₄ are each independently CR²; or

where for formula (1B):
X⁴ is C or N; and
one or two of X₁, X₂ and X₃ are independently selected from N and NR⁸, and the remainder are each independently CR²;
such that X₁, X₂, X₃, X₄ and N form a heteroaryl group;

each R² is independently selected from H, alkyl, CN, halo, heteroaryl, heterocycloalkyl, cycloalkyl, OR⁴, CONR⁵R⁶ and SO₂R⁷, wherein said alkyl, heteroaryl, heterocycloalkyl and cycloalkyl groups are each optionally further substituted by one or more groups selected from alkyl, halo and OR⁹;

each R⁸ is independently selected from H and alkyl, wherein said alkyl group is optionally further substituted by one or more groups selected from CN, halo, heteroaryl, heterocycloalkyl, cycloalkyl, OR⁴, CONR⁵R⁶ and SO₂R⁷;

R⁴, R⁵, R⁶, R⁷ and R⁹ are each independently selected from H and alkyl; or

R⁵ and R⁶ together with the nitrogen to which they are attached are linked to form a cyclic group which optionally further comprises one or more heteroatoms selected from O, N and S;

with the proviso that when the compound is of formula (IB), the compound is other than 3-(5-isopropyl-4H-1,2,4-triazol-3-yl)-N-(tetrahydro-2H-pyran-4-yl)-1H-pyrazolo[4,3-c]pyridin-4-amine or 3-(5-cyclopropyl-4H-1,2,4-triazol-3-yl)-N-(tetrahydro-2H-pyran-4-yl)-1H-pyrazolo[4,3-c]pyridin-4-amine.

Another aspect of the invention relates to a compound of formula (1A) or (1B), or a pharmaceutically acceptable salt thereof,

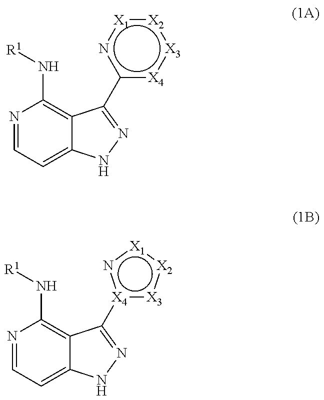

wherein:

R¹ is a group selected from alkyl, monocyclic heterocycloalkyl, bicyclic heterocycloalkyl and cycloalkyl, each of which is optionally substituted by one or more groups selected from alkyl, halo and cycloalkyl;

where for formula (1A):
one, two or three of X₁, X₂, X₃ and X₄ are N, and the remainder are each independently CR²; or

where for formula (1B):
X⁴ is C or N; and
one or two of X₁, X₂ and X₃ are independently selected from N and NR⁸, and the remainder are each independently CR²;
such that X₁, X₂, X₃, X₄ and N form a heteroaryl group;

each R² is independently selected from H, alkyl, CN, halo, heteroaryl, heterocycloalkyl, cycloalkyl, OR⁴, CONR⁵R⁶ and SO₂R⁷, wherein said alkyl, heteroaryl, heterocycloalkyl and cycloalkyl groups are each optionally further substituted by one or more groups selected from alkyl, halo and OR⁹;

each R⁸ is independently selected from H and alkyl, wherein said alkyl group is optionally further substituted by one or more groups selected from CN, halo, heteroaryl, heterocycloalkyl, cycloalkyl, OR⁴, CONR⁵R⁶ and SO₂R⁷;

R⁴, R⁵, R⁶, R⁷ and R⁹ are each independently selected from H and alkyl; or

R⁵ and R⁶ together with the nitrogen to which they are attached are linked to form a cyclic group which optionally further comprises one or more heteroatoms selected from O, N and S;

with the proviso that when the compound is of formula (IB), the compound is other than 3-(5-isopropyl-4H-1,2,4-triazol-3-yl)-N-(tetrahydro-2H-pyran-4-yl)-1H-pyrazolo[4,3-c]pyridin-4-amine or 3-(5-cyclopropyl-4H-1,2,4-triazol-3-yl)-N-(tetrahydro-2H-pyran-4-yl)-1H-pyrazolo[4,3-c]pyridin-4-amine.

A second aspect of the invention relates to a pharmaceutical composition comprising at least one compound as described above and a pharmaceutically acceptable carrier, diluent or excipient.

A third aspect of the invention relates to a compound as described above for use in medicine.

A fourth aspect of the invention relates to a compound as described above for use in treating a disorder selected from cancer and neurodegenerative diseases such as Parkinson's Disease.

A fifth aspect of the invention relates to the use of a compound as described above in the preparation of a medicament for treating or preventing a disorder selected from cancer and neurodegenerative diseases such as Parkinson's Disease.

A sixth aspect of the invention relates to the use of a compound as described above in the preparation of a medicament for the prevention or treatment of a disorder caused by, associated with or accompanied by any abnormal kinase activity wherein the kinase is LRRK2.

A seventh aspect of the invention relates to a method of treating a mammal having a disease state alleviated by inhibition of a kinase such as LRRK2, wherein the method comprises administering to a mammal a therapeutically effective amount of a compound as described above.

An eighth aspect of the invention relates to the use of a compound as described above in an assay for identifying further candidate compounds capable of inhibition of a kinase such as LRRK2.

A ninth aspect of the invention relates to a process for preparing a compound as described above.

### DETAILED DESCRIPTION

The present invention relates to pyrazolopyridine compounds that are capable of inhibiting one or more kinases, more particularly LRRK, even more particularly LRRK2. Specifically, the invention relates to substituted pyrazolo[4,3-c]pyridine derivatives.

The presently claimed compounds (i) are unsubstituted at the 7-position of the pyrazolo[4,3-c]pyridine core; (ii) have an amine group at the 4-position; and (iii) have an optionally substituted heteroaryl group at the 3-position oriented such that a hydrogen bond may form between a nitrogen of the heteroaryl group and the hydrogen of the amine group in the 4-position. The hydrogen bonding arrangement is shown below:

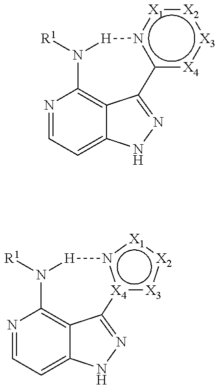

Advantageously, the presently claimed compounds exhibit unexpectedly high LRRK2 affinity compared to compounds described in the art to date. In particular, it is believed that this particular arrangement of hydrogen bonding and substitution provides unexpectedly higher affinity values compared to analogous compounds that are substituted at the 7-position and/or have a heteroaryl in the 3-position that is not oriented in the correct position to form a hydrogen bond with the hydrogen of the amine group in the 4-position.

“Alkyl” is defined herein as a straight-chain or branched alkyl radical, for example, methyl, ethyl, propyl, isopropyl, butyl, isobutyl, tert-butyl, pentyl or hexyl. Specific examples are methyl, ethyl and isopropyl. In certain embodiment the alkyl group is a C₁₋₂₀ alkyl group, more preferably a C₁₋₁₅ and in some embodiments, a C₁₋₁₂ alkyl group, and in still other embodiments, a C₁₋₁₀ alkyl group, a C₁₋₆ alkyl group or a C₁₋₃ alkyl group. A particular “alkyl” group is C₁₋₆-alkyl.

“Cycloalkyl” is defined herein as a monocyclic alkyl ring, such as, cyclopropyl, cyclobutyl, cyclopentyl, cyclohexyl or cycloheptyl, or a fused bicyclic ring system such as norbornane. Specific examples are cyclopropyl and cyclohexyl. The cycloalkyl group may be a C₃₋₁₂ cycloalkyl group, such as a C₃₋₆ cycloalkyl group.

“Halogen” or “halo” is defined herein as chloro, fluoro, bromo or iodo. A specific “halo” is chloro (Cl).

As used herein, the term “aryl” refers to a C₆₋₁₂ aromatic group, which may be benzocondensed, for example, phenyl or naphthyl.

“Heteroaryl” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc. Specific examples are pyrazolyl and pyridinyl.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. In certain embodiments the heterocycloalkyl group is a C₃₋₇-heterocycloalkyl, more preferably a C₃₋₆-heterocycloalkyl. Alternatively, the heterocycloalkyl group is a C₄₋₇-heterocycloalkyl, such as a C₄₋₆-heterocycloalkyl. In certain embodiments heterocycloalkyl groups include, but are not limited to, piperazinyl, piperidinyl, morpholinyl, thiomorpholinyl, pyrrolidinyl, tetrahydrofuranyl and tetrahydropyranyl. Specific examples are azetidinyl, morpholinyl, tetrahydropyranyl, tetrahydrofuranyl, pyrrolidinyl and piperidinyl.

In one embodiment, the compound of the invention is of formula (1B), but with the proviso that when X¹ and X³ are N, then X² is other than carbon substituted with isopropyl or cyclopropyl.

In one embodiment, the invention relates to a compound of formula (1A), wherein X₄ is N and X₁, X₂ and X₃ are each independently CR².

In one embodiment, the invention relates to a compound of formula (1A), wherein X₂ is N and X₁, X₃ and X₄ are each independently CR².

In one embodiment, the invention relates to a compound of formula (1A), wherein X₁, X₂, X₃ and X₄ are each independently CR².

In one embodiment, the invention relates to a compound of formula (1A) wherein X₄ is N, X₁ and X₂ are CH, and X₃ is CR².

In one embodiment, the invention relates to a compound of formula (1A) wherein X₂ is N, X₁ and X₄ are CH, and X₃ is CR².

In one embodiment, the invention relates to a compound of formula (1A) wherein X₁, X₂ and X₄ are CH and X₃ is CR².

In a certain embodiment the invention relates to a compound of formula (1A) wherein X₁ is CR² and R² is H, methyl or morpholinyl.

In a certain embodiment the invention relates to a compound of formula (1A) wherein X₁ is CR² and R² is H.

In a certain embodiment the invention relates to a compound of formula (1A) wherein X₁ is CR² and R² is methyl.

In a certain embodiment the invention relates to a compound of formula (1A) wherein X₁ is CR² and R² is morpholinyl.

In a certain embodiment the invention relates to a compound of formula (1A) wherein X₁ is CR² and R² is H, methyl or morpholinyl and X₂ is CH.

In a certain embodiment the invention relates to a compound of formula (1A) wherein X₁ is CR² and R² is H, methyl or morpholinyl and X₂ is N.

In a certain embodiment the invention relates to a compound of formula (1A) wherein X₁ is CR² wherein R² is H, methyl or morpholinyl, X₂ is CH and X₃ is CR² wherein R² is (3R,4R)-3,4-difluoropyrrolidin-1-yl, 1,3-dimethyl-1H-pyrazol-4-yl, 1,5-dimethyl-1H-pyrazol-4-yl, 1-methyl-1H-pyrazol-3-yl, 1-methyl-1H-pyrazol-4-yl, 1-methyl-1H-pyrazol-5-yl, 1-methylpiperidin-4-yl, 2,2,6,6-tetrafluoromorpholino, 2-fluoro-pyridin-3-yl, —C(O)-azetidin-1-yl, —C(O)-morpholinyl, —C(O)N(CH₃)₂, —CF₃, —Cl, —CN, ethoxy, H, morpholinyl, —SO₂-isopropyl, —SO₂CH₃, tetrahydro-2H-pyran-4-yl, tetrahydrofuran-2-yl or tetrahydrofuran-3-yl.

In a certain embodiment the invention relates to a compound of formula (1A) wherein X₁ is CR² wherein R² is H, methyl or morpholinyl, X₂ is CH and X₃ is CH.

In a certain embodiment the invention relates to a compound of formula (1A) wherein X₁ is CR² wherein R² is H, methyl or morpholinyl, X₂ is CH, X₃ is CR² wherein R² is (3R,4R)-3,4-difluoropyrrolidin-1-yl, 1,3-dimethyl-1H-pyrazol-4-yl, 1,5-dimethyl-1H-pyrazol-4-yl, 1-methyl-1H-pyrazol-3-yl, 1-methyl-1H-pyrazol-4-yl, 1-methyl-1H-pyrazol-5-yl, 1-methylpiperidin-4-yl, 2,2,6,6-tetrafluoromorpholino, 2-fluoro-pyridin-3-yl, —C(O)-azetidin-1-yl, —C(O)-morpholinyl, —C(O)N(CH₃)₂, —CF₃, —Cl, —CN, ethoxy, H, morpholinyl, —SO₂-isopropyl, —SO₂CH₃, tetrahydro-2H-pyran-4-yl, tetrahydrofuran-2-yl or tetrahydrofuran-3-yl and X₄ is N.

In a certain embodiment the invention relates to a compound of formula (1A) wherein X₁ is CR² wherein R² is H, methyl or morpholinyl, X₂ is CH, X₃ is CH and X₄ is N.

In a certain embodiment the invention relates to a compound of formula (1A) wherein X₁ is CR² wherein R² is H, methyl or morpholinyl, X₂ is CH, X₃ is CR² wherein R² is (3R,4R)-3,4-difluoropyrrolidin-1-yl, 1,3-dimethyl-1H-pyrazol-4-yl, 1,5-dimethyl-1H-pyrazol-4-yl, 1-methyl-1H-pyrazol-3-yl, 1-methyl-1H-pyrazol-4-yl, 1-methyl-1H-pyrazol-5-yl, 1-methylpiperidin-4-yl, 2,2,6,6-tetrafluoromorpholino, 2-fluoro-pyridin-3-yl, —C(O)-azetidin-1-yl, —C(O)-morpholinyl, —C(O)N(CH₃)₂, —CF₃, —Cl, —CN, ethoxy, H, morpholinyl, —SO₂-isopropyl, —SO₂CH₃, tetrahydro-2H-pyran-4-yl, tetrahydrofuran-2-yl or tetrahydrofuran-3-yl and X₄ is CH.

In a certain embodiment the invention relates to a compound of formula (1A) wherein X₁ is CR² wherein R² is H, methyl or morpholinyl, X₂ is CH, X₃ is CH and X₄ is CH.

In one embodiment, the invention relates to a compound of formula (1B), wherein X⁴ is C, X₁ is NR⁸, and X₂ and X₃ are each independently CR².

In one embodiment, the invention relates to a compound of formula (1B), wherein X⁴ is C, X₃ is CR², one of X₁ and X₂ is N and the other is NR⁸.

In one embodiment, the invention relates to a compound of formula (1B), wherein X⁴ is C, X₂ is NR⁸, and X₁ and X₃ are each independently CR².

In one embodiment, the invention relates to a compound of formula (1B), wherein X⁴ is N, and X₁, X₂ and X₃ are each independently CR².

In one embodiment, the invention relates to a compound of formula (1B), wherein X⁴ is C, X₂ is CR², one of X₁ and X₃ is N and the other is NR⁸.

In one embodiment, the invention relates to a compound of formula (1B), wherein X⁴ is C, X₁ is NR⁸, and X₃ is CH and X₂ is CR².

In one embodiment, the invention relates to a compound of formula (1B), wherein X⁴ is C, X₃ is CH, X₁ is N and X₂ is NR⁸.

In one embodiment, the invention relates to a compound of formula (1B), wherein X⁴ is C, X₂ is NR⁸, and X₁ and X₃ are each independently CH.

In one embodiment, the invention relates to a compound of formula (1B), wherein X⁴ is N, X₁, and X₃ are CH and X₂ is CR².

In one embodiment, the invention relates to a compound of formula (1B), wherein X⁴ is C, X₂ is CR², one of X₁ and X₃ is N and the other is NR⁸.

In a certain embodiment the invention relates to a compound of formula (1B) wherein X₁ is CH, X₂ is CF₃, X₃ is CH and X₄ is N.

In a certain embodiment the invention relates to a compound of formula (1B) wherein X₁ is N, X₂ is C-cyclopropyl, X₃ is N—CH₃ and X₄ is C.

In a certain embodiment the invention relates to a compound of formula (1B) wherein X₁ is N—CH₃, X₂ is C-cyclopropyl or C-ethyl, X₃ is N or CH and X₄ is C.

In a certain embodiment the invention relates to a compound of formula (1B) wherein X₁ is N—CH₃, X₂ is C-cyclopropyl, X₃ is N and X₄ is C.

In a certain embodiment the invention relates to a compound of formula (1B) wherein X₁ is N—CH₃, X₂ is C-cyclopropyl, X₃ is CH and X₄ is C.

In a certain embodiment the invention relates to a compound of formula (1B) wherein X₁ is N—CH₃, X₂ is C-ethyl, X₃ is N and X₄ is C.

In a certain embodiment the invention relates to a compound of formula (1B) wherein X₁ is N—CH₃, X₂ is C-ethyl, X₃ is CH and X₄ is C.

In one embodiment of the invention, R¹ is:
(i) an alkyl group optionally substituted by one or more halo or cycloalkyl groups;
(ii) a monocyclic heterocycloalkyl or bicyclic heterocycloalkyl group optionally substituted by one or more alkyl groups; or
(iii) a cycloalkyl group optionally substituted by one or more halo groups.

In one embodiment of the invention, R¹ is:
(i) an isopropyl or ethyl group, each of which is optionally substituted by a group selected from halo and cyclopropyl;
(ii) a tetrahydropyranyl group or 8-oxabicyclo[3.2.1]octan-3-yl group, each of which is optionally substituted by one or more alkyl groups; or
(ii) a cyclohexyl group optionally substituted by one or more halo groups.

In a certain embodiment the invention relates to a compound of formula (1A) wherein R¹ is 2,2,2-trifluoroethyl, 2,6-dimethyltetrahydro-2H-pyran-4-yl, 4,4-difluorocyclohexyl, 8-oxabicyclo[3.2.1]octan-3-yl, cyclopropylmethyl, dicyclopropylmethyl, ethyl, isopropyl or tetrahydro-2H-pyran-4-yl.

In a certain embodiment the invention relates to a compound of formula (1A) wherein R¹ is 2,2,2-trifluoroethyl.

In a certain embodiment the invention relates to a compound of formula (1A) wherein R¹ is 2,6-dimethyltetrahydro-2H-pyran-4-yl.

In a certain embodiment the invention relates to a compound of formula (1A) wherein R¹ is 4,4-difluorocyclohexyl.

In a certain embodiment the invention relates to a compound of formula (1A) wherein R¹ is 8-oxabicyclo[3.2.1]octan-3-yl.

In a certain embodiment the invention relates to a compound of formula (1A) wherein R¹ is cyclopropylmethyl.

In a certain embodiment the invention relates to a compound of formula (1A) wherein R¹ is dicyclopropylmethyl.

In a certain embodiment the invention relates to a compound of formula (1A) wherein R¹ is ethyl.

In a certain embodiment the invention relates to a compound of formula (1A) wherein R¹ isopropyl.

In a certain embodiment the invention relates to a compound of formula (1A) wherein R¹ is tetrahydro-2H-pyran-4-yl.

In a certain embodiment the invention relates to a compound of formula (1B) wherein R¹ is ethyl, isopropyl or tetrahydro-2H-pyran-4-yl.

In a certain embodiment the invention relates to a compound of formula (1B) wherein R¹ is ethyl.

In a certain embodiment the invention relates to a compound of formula (1B) wherein R¹ isopropyl.

In a certain embodiment the invention relates to a compound of formula (1B) wherein R¹ is tetrahydro-2H-pyran-4-yl.

In one embodiment of the invention, R² is:
(i) an alkyl group optionally substituted by one or more halo or OR⁴ groups, where R⁴ is alkyl;
(ii) a heteroaryl group optionally substituted by one or more groups selected from alkyl and halo;
(iii) a heterocycloalkyl group optionally substituted by one or more groups selected from alkyl and halo;
(iv) a cycloalkyl group optionally substituted by one or more groups selected from alkyl and halo;
(v) CN;
(vi) halo;
(vii) SO₂R⁷ where R⁷ is alkyl;
(viii) OR⁴, where R⁴ is alkyl; or
(ix) CONR⁵R⁶ where R⁵ and R⁶ are both alkyl, or R⁵ and R⁶ together with the nitrogen to which they are attached are linked to form a 4, 5 or 6-membered heterocycloalkyl group.

In one embodiment of the invention, R² is:
(i) a methyl, ethyl or isopropyl group (such as a methyl or ethyl group) optionally substituted by one or more halo or OR⁴ groups, where R⁴ is alkyl;
(ii) a heteroaryl group selected from a pyrazolyl group and a pyridinyl group, each of which is optionally substituted by one or more alkyl or halo groups;
(iii) a heterocycloalkyl group selected from morpholinyl, tetrahydropyranyl, piperidinyl and tetrahydrofuranyl, each of which is optionally substituted by one or more alkyl or halo groups;
(iv) a cyclopropyl group optionally substituted by one or more alkyl or halo groups;
(v) CN;
(vi) halo;
(vii) SO₂R⁷ where R⁷ is methyl or isopropyl;
(viii) OR⁴, where R⁴ is ethyl; or
(ix) CONR⁵R⁶ where R⁵ and R⁶ are both methyl, or R⁵ and R⁶ together with the nitrogen to which they are attached are linked to form a morpholinyl group, or R⁵ and R⁶ together with the nitrogen to which they are attached are linked to form an azetidinyl group.

In one embodiment, the compound of the invention is selected from the following:

| | Name | Structure |
|---|---|---|
| 1 | 3-(5-ethyl-1-methyl-1H- pyrazol-3-yl)-N-(tetrahydro- 2H-pyran-4-yl)-1H- pyrazolo[4,3-c]pyridin-4- amine | 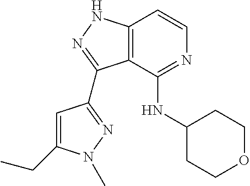 [SMILES: CCc1cc(-c2n[nH]c3ccnc(NC4CCOCC4)c23)nn1C] |
| 2 | N-isopropyl-3-(4- morpholinopyrimidin-2-yl)- 1H-pyrazolo[4,3-c]pyridin-4- amine | 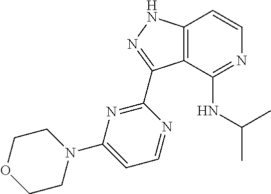 [SMILES: CC(C)Nc1nccc2[nH]nc(-c3nccc(N4CCOCC4)n3)c12] |
| 3 | N-isopropyl-3-(4- morpholinopyridin-2-yl)-1H- pyrazolo[4,3-c]pyridin-4- amine | 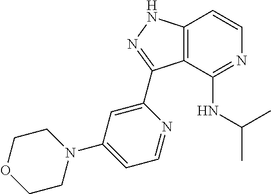 [SMILES: CC(C)Nc1nccc2[nH]nc(-c3cc(N4CCOCC4)ccn3)c12] |
| 4 | N-isopropyl-3-(6- morpholinopyrimidin-4-yl)- 1H-pyrazolo[4,3-c]pyridin-4- amine | 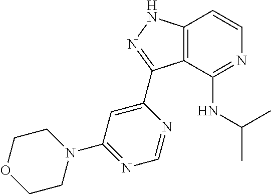 [SMILES: CC(C)Nc1nccc2[nH]nc(-c3cc(N4CCOCC4)ncn3)c12] |
| 5 | 3-(1-isopropyl-1H-1,2,3- triazol-4-yl)-N-(tetrahydro- 2H-pyran-4-yl)-1H- pyrazolo[4,3-c]pyridin-4- amine | 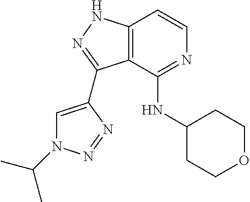 [SMILES: CC(C)n1cc(-c2n[nH]c3ccnc(NC4CCOCC4)c23)nn1] |
| 6 | 3-(1-isopropyl-1H-imidazol- 4-yl)-N-(tetrahydro-2H- pyran-4-yl)-1H-pyrazolo[4,3- c]pyridin-4-amine | 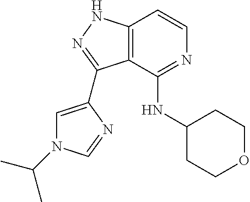 [SMILES: CC(C)n1cnc(-c2n[nH]c3ccnc(NC4CCOCC4)c23)c1] |
| 7 | N-isopropyl-3-(1-isopropyl- 1H-1,2,3-triazol-4-yl)-1H- pyrazolo[4,3-c]pyridin-4- amine | 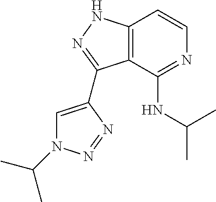 [SMILES: CC(C)Nc1nccc2[nH]nc(-c3cn(C(C)C)nn3)c12] |
| 8 | N-isopropyl-3-(1-methyl- 1H-1,2,3-triazol-4-yl)-1H- pyrazolo[4,3-c]pyridin-4- amine | 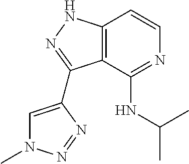 [SMILES: CC(C)Nc1nccc2[nH]nc(-c3cn(C)nn3)c12] |
| 9 | N-ethyl-3-(1-isopropyl-1H- 1,2,3-triazol-4-yl)-1H- pyrazolo[4,3-c]pyridin-4- amine | 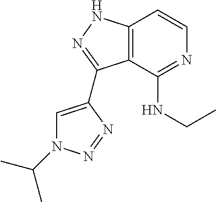 [SMILES: CCNc1nccc2[nH]nc(-c3cn(C(C)C)nn3)c12] |
| 10 | N-ethyl-3-(1-methyl-1H- 1,2,3-triazol-4-yl)-1H- pyrazolo[4,3-c]pyridin-4- amine | 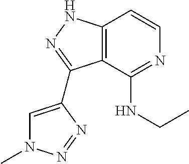 [SMILES: CCNc1nccc2[nH]nc(-c3cn(C)nn3)c12] |
| 11 | 3-(5-ethyl-1-methyl-1H- pyrazol-3-yl)-N-isopropyl- 1H-pyrazolo[4,3-c]pyridin-4- amine | 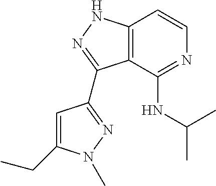 [SMILES: CCc1cc(-c2n[nH]c3ccnc(NC(C)C)c23)nn1C] |
| 12 | N-ethyl-3-(1-methyl-1H- imidazol-4-yl)-1H- pyrazolo[4,3-c]pyridin-4- amine | 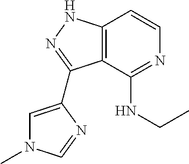 [SMILES: CCNc1nccc2[nH]nc(-c3cn(C)cn3)c12] |
| 13 | N-isopropyl-3-(pyridin-2- yl)-1H-pyrazolo[4,3- c]pyridin-4-amine | 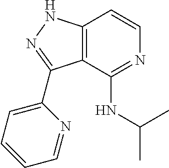 [SMILES: CC(C)Nc1nccc2[nH]nc(-c3ccccn3)c12] |
| 14 | N-ethyl-3-(1-(2- methoxyethyl)-1H-1,2,3- triazol-4-yl)-1H- pyrazolo[4,3-c]pyridin-4- amine | 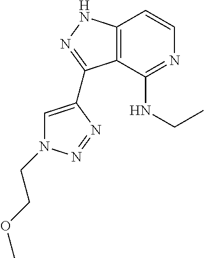 [SMILES: CCNc1nccc2[nH]nc(-c3cn(CCOC)nn3)c12] |
| 15 | N-isopropyl-3-(pyrimidin-4- yl)-1H-pyrazolo[4,3- c]pyridin-4-amine | 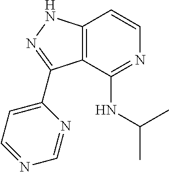 [SMILES: CC(C)Nc1nccc2[nH]nc(-c3ccncn3)c12] |
| 16 | N-ethyl-3-(pyridin-2-yl)-1H- pyrazolo[4,3-c]pyridin-4- amine | 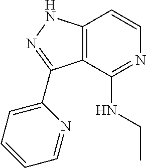 [SMILES: CCNc1nccc2[nH]nc(-c3ccccn3)c12] |
| 17 | 3-(pyridin-2-yl)-N- (tetrahydro-2H-pyran-4-yl)- 1H-pyrazolo[4,3-c]pyridin-4- amine | 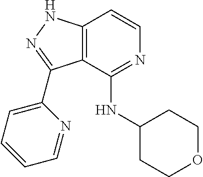 [SMILES: c1ccc(-c2n[nH]c3ccnc(NC4CCOCC4)c23)nc1] |
| 18 | 3-(pyrimidin-4-yl)-N- (tetrahydro-2H-pyran-4-yl)- 1H-pyrazolo[4,3-c]pyridin-4- amine | 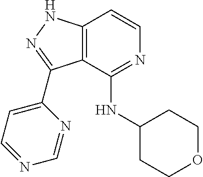 [SMILES: c1cc(-c2n[nH]c3ccnc(NC4CCOCC4)c23)ncn1] |
| 19 | N-ethyl-3-(pyrimidin-4-yl)- 1H-pyrazolo[4,3-c]pyridin-4- amine | 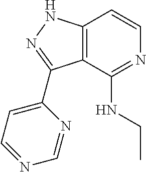 [SMILES: CCNc1nccc2[nH]nc(-c3ccncn3)c12] |
| 20 | N-isopropyl-3-(4- (trifluoromethyl)pyridin-2- yl)-1H-pyrazolo[4,3- c]pyridin-4-amine | 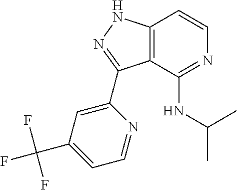 [SMILES: CC(C)Nc1nccc2[nH]nc(-c3cc(C(F)(F)F)ccn3)c12] |
| 21 | N-ethyl-3-(4- (trifluoromethyl)pyridin-2- yl)-1H-pyrazolo[4,3- c]pyridin-4-amine | 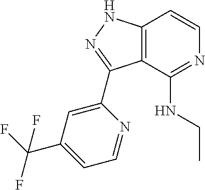 [SMILES: CCNc1nccc2[nH]nc(-c3cc(C(F)(F)F)ccn3)c12] |
| 22 | N-(tetrahydro-2H-pyran-4- yl)-3-(6- (trifluoromethyl)pyrimidin-4- yl)-1H-pyrazolo[4,3- c]pyridin-4-amine | 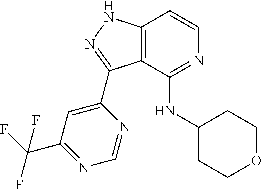 [SMILES: FC(F)(F)c1cc(-c2n[nH]c3ccnc(NC4CCOCC4)c23)ncn1] |
| 23 | N-(tetrahydro-2H-pyran-4- yl)-3-(4- (trifluoromethyl)pyridin-2- yl)-1H-pyrazolo[4,3- c]pyridin-4-amine | 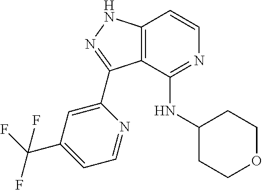 [SMILES: FC(F)(F)c1ccnc(-c2n[nH]c3ccnc(NC4CCOCC4)c23)c1] |
| 24 | N-isopropyl-3-(6- (trifluoromethyl)pyrimidin-4- yl)-1H-pyrazolo[4,3- c]pyridin-4-amine | 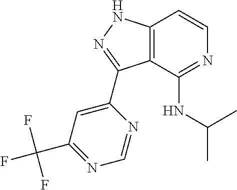 [SMILES: CC(C)Nc1nccc2[nH]nc(-c3cc(C(F)(F)F)ncn3)c12] |
| 25 | 2-(4-(ethylamino)-1H- pyrazolo[4,3-c]pyridin-3- yl)isonicotinonitrile | 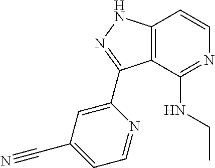 [SMILES: CCNc1nccc2[nH]nc(-c3cc(C#N)ccn3)c12] |
| 26 | 2-(4-((tetrahydro-2H-pyran- 4-yl)amino)-1H- pyrazolo[4,3-c]pyridin-3- yl)isonicotinonitrile | 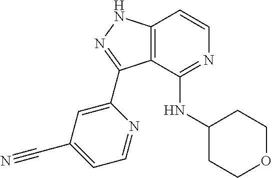 [SMILES: N#Cc1ccnc(-c2n[nH]c3ccnc(NC4CCOCC4)c23)c1] |
| 27 | N-ethyl-3-(6- (trifluoromethyl)pyrimidin-4- yl)-1H-pyrazolo[4,3- c]pyridin-4-amine | 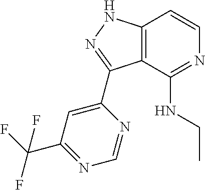 [SMILES: CCNc1nccc2[nH]nc(-c3cc(C(F)(F)F)ncn3)c12] |
| 28 | 3-(6-methylpyridin-2-yl)-N- (tetrahydro-2H-pyran-4-yl)- 1H-pyrazolo[4,3-c]pyridin-4- amine | 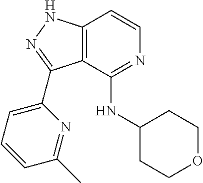 [SMILES: Cc1cccc(-c2n[nH]c3ccnc(NC4CCOCC4)c23)n1] |
| 29 | 3-(4-chloropyridin-2-yl)-N- (tetrahydro-2H-pyran-4-yl)- 1H-pyrazolo[4,3-c]pyridin-4- amine | 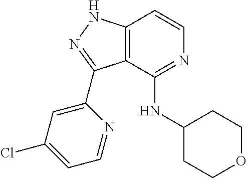 [SMILES: Clc1ccnc(-c2n[nH]c3ccnc(NC4CCOCC4)c23)c1] |
| 30 | 3-(4-chloropyridin-2-yl)-N- isopropyl-1H-pyrazolo[4,3- c]pyridin-4-amine | 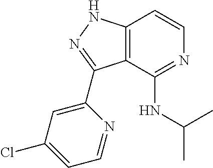 [SMILES: CC(C)Nc1nccc2[nH]nc(-c3cc(Cl)ccn3)c12] |
| 31 | 2-(4-(isopropylamino)-1H- pyrazolo[4,3-c]pyridin-3- yl)isonicotinonitrile | 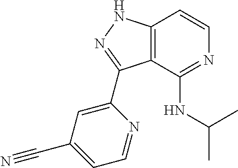 [SMILES: CC(C)Nc1nccc2[nH]nc(-c3cc(C#N)ccn3)c12] |
| 32 | N-isopropyl-3-(4- (trifluoromethyl)-1H- pyrazol-1-yl)-1H- pyrazolo[4,3-c]pyridin-4- amine | 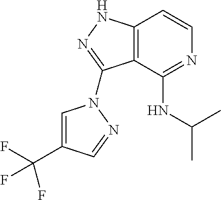 [SMILES: CC(C)Nc1nccc2[nH]nc(-n3cc(C(F)(F)F)cn3)c12] |
| 33 | 3-(2-methylpyrimidin-4-yl)- N-(tetrahydro-2H-pyran-4- yl)-1H-pyrazolo[4,3- c]pyridin-4-amine | 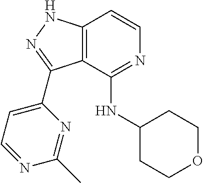 [SMILES: Cc1nccc(-c2n[nH]c3ccnc(NC4CCOCC4)c23)n1] |
| 34 | N-(4,4-difluorocyclohexyl)- 3-(pyridin-2-yl)-1H- pyrazolo[4,3-c]pyridin-4- amine | 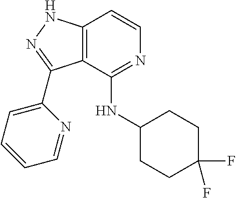 [SMILES: FC1(F)CCC(Nc2nccc3[nH]nc(-c4ccccn4)c23)CC1] |
| 35 | N-isopropyl-3-(4-(1-methyl- 1H-pyrazol-4-yl)pyridin-2- yl)-1H-pyrazolo[4,3- c]pyridin-4-amine | 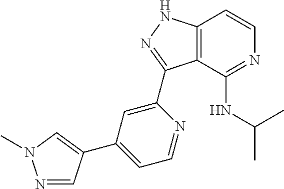 [SMILES: CC(C)Nc1nccc2[nH]nc(-c3cc(-c4cnn(C)c4)ccn3)c12] |
| 36 | N-isopropyl-3-(4-(1-methyl- 1H-pyrazol-5-yl)pyridin-2- yl)-1H-pyrazolo[4,3- c]pyridin-4-amine | 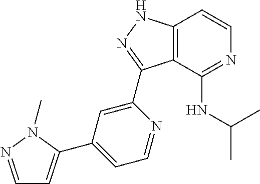 [SMILES: CC(C)Nc1nccc2[nH]nc(-c3cc(-c4ccnn4C)ccn3)c12] |
| 37 | 3-(2-fluoro-[3,4′-bipyridin]- 2′-yl)-N-isopropyl-1H- pyrazolo[4,3-c]pyridin-4- amine | 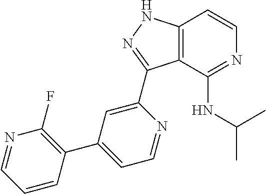 [SMILES: CC(C)Nc1nccc2[nH]nc(-c3cc(-c4cccnc4F)ccn3)c12] |
| 38 | N-(4,4-difluorocyclohexyl)- 3-(pyrimidin-4-yl)-1H- pyrazolo[4,3-c]pyridin-4- amine | 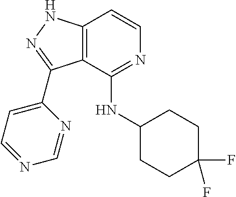 [SMILES: FC1(F)CCC(Nc2nccc3[nH]nc(-c4ccncn4)c23)CC1] |
| 39 | N-isopropyl-3-(4- (tetrahydro-2H-pyran-4- yl)pyridin-2-yl)-1H- pyrazolo[4,3-c]pyridin-4- amine | 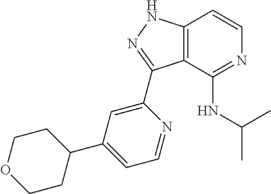 [SMILES: CC(C)Nc1nccc2[nH]nc(-c3cc(C4CCOCC4)ccn3)c12] |
| 40 | N-isopropyl-3-(4-(1- methylpiperidin-4-yl)pyridin- 2-yl)-1H-pyrazolo[4,3- c]pyridin-4-amine | 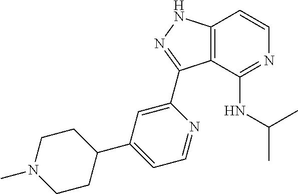 [SMILES: CC(C)Nc1nccc2[nH]nc(-c3cc(C4CCN(C)CC4)ccn3)c12] |
| 41 | 3-(5-cyclopropyl-4-methyl- 4H-1,2,4-triazol-3-yl)-N- isopropyl-1H-pyrazolo[4,3- c]pyridin-4-amine | 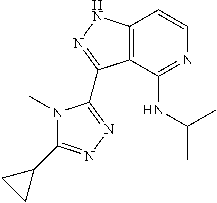 [SMILES: CC(C)Nc1nccc2[nH]nc(-c3nnc(C4CC4)n3C)c12] |
| 42 | 3-(5-cyclopropyl-1-methyl- 1H-1,2,4-triazol-3-yl)-N- isopropyl-1H-pyrazolo[4,3- c]pyridin-4-amine | 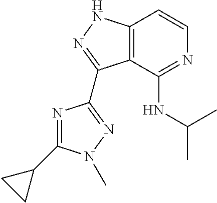 [SMILES: CC(C)Nc1nccc2[nH]nc(-c3nc(C4CC4)n(C)n3)c12] |
| 43 | 3-(pyridin-2-yl)-N-(2,2,2- trifluoroethyl)-1H- pyrazolo[4,3-c]pyridin-4- amine | 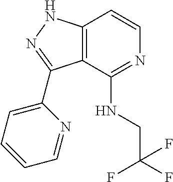 [SMILES: FC(F)(F)CNc1nccc2[nH]nc(-c3ccccn3)c12] |
| 44 | 3-(4-(1,5-dimethyl-1H- pyrazol-4-yl)pyridin-2-yl)-N- isopropyl-1H-pyrazolo[4,3- c]pyridin-4-amine | 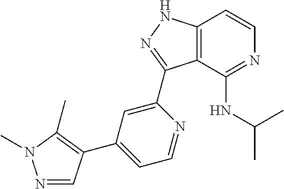 [SMILES: Cc1c(-c2ccnc(-c3n[nH]c4ccnc(NC(C)C)c34)c2)cnn1C] |
| 45 | 3-(4-(1,3-dimethyl-1H- pyrazol-4-yl)pyridin-2-yl)-N- isopropyl-1H-pyrazolo[4,3- c]pyridin-4-amine | 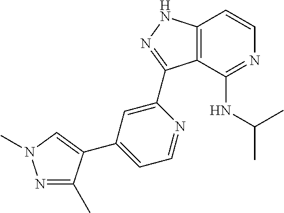 [SMILES: Cc1nn(C)cc1-c1ccnc(-c2n[nH]c3ccnc(NC(C)C)c23)c1] |
| 46 | N-isopropyl-3-(4-(1-methyl- 1H-pyrazol-3-yl)pyridin-2- yl)-1H-pyrazolo[4,3- c]pyridin-4-amine | 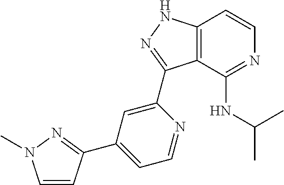 [SMILES: CC(C)Nc1nccc2[nH]nc(-c3cc(-c4ccn(C)n4)ccn3)c12] |
| 47 | N-isopropyl-3-(6- (tetrahydro-2H-pyran-4- yl)pyrimidin-4-yl)-1H- pyrazolo[4,3-c]pyridin-4- amine | 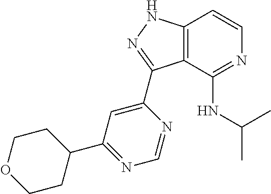 [SMILES: CC(C)Nc1nccc2[nH]nc(-c3cc(C4CCOCC4)ncn3)c12] |
| 48 | N-isopropyl-3-(6- (tetrahydrofuran-3- yl)pyrimidin-4-yl)-1H- pyrazolo[4,3-c]pyridin-4- amine | 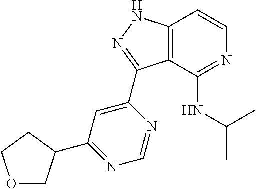 [SMILES: CC(C)Nc1nccc2[nH]nc(-c3cc(C4CCOC4)ncn3)c12] |
| 49 | N-isopropyl-3-(4- (tetrahydrofuran-3- yl)pyridin-2-yl)-1H- pyrazolo[4,3-c]pyridin-4- amine | 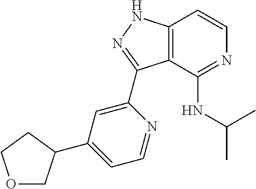 [SMILES: CC(C)Nc1nccc2[nH]nc(-c3cc(C4CCOC4)ccn3)c12] |
| 50 | 3-(4-ethoxypyridin-2-yl)-N- (tetrahydro-2H-pyran-4-yl)- 1H-pyrazolo[4,3-c]pyridin-4- amine | 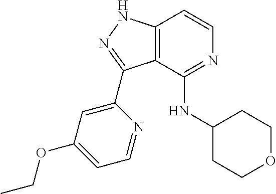 [SMILES: CCOc1ccnc(-c2n[nH]c3ccnc(NC4CCOCC4)c23)c1] |
| 51 | 6-methyl-N-(tetrahydro-2H- pyran-4-yl)-3-(4- (trifluoromethyl)pyridin-2- yl)-1H-pyrazolo[4,3- c]pyridin-4-amine | 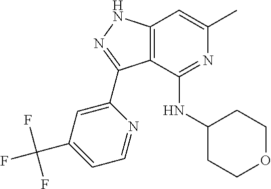 [SMILES: Cc1cc2[nH]nc(-c3cc(C(F)(F)F)ccn3)c2c(NC2CCOCC2)n1] |
| 52 | N,N-dimethyl-2-(4- ((tetrahydro-2H-pyran-4- yl)amino)-1H-pyrazolo[4,3- c]pyridin-3- yl)isonicotinamide | 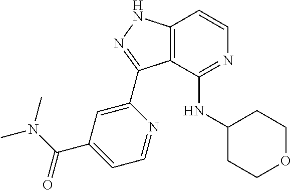 [SMILES: CN(C)C(=O)c1ccnc(-c2n[nH]c3ccnc(NC4CCOCC4)c23)c1] |
| 53 | N-isopropyl-3-(4- (tetrahydrofuran-2- yl)pyridin-2-yl)-1H- pyrazolo[4,3-c]pyridin-4- amine | 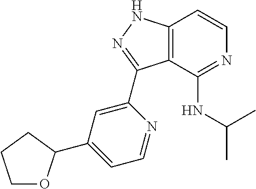 [SMILES: CC(C)Nc1nccc2[nH]nc(-c3cc(C4CCCO4)ccn3)c12] |
| 54 | N-isopropyl-3-(6- (tetrahydrofuran-2- yl)pyrimidin-4-yl)-1H- pyrazolo[4,3-c]pyridin-4- amine | 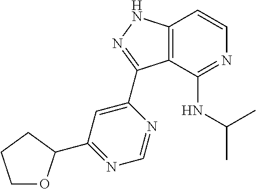 [SMILES: CC(C)Nc1nccc2[nH]nc(-c3cc(C4CCCO4)ncn3)c12] |
| 55 | 2-(4-(isopropylamino)-1H- pyrazolo[4,3-c]pyridin-3-yl)- N,N- dimethylisonicotinamide | 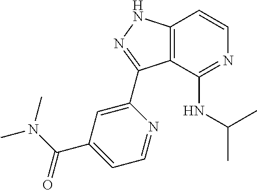 [SMILES: CC(C)Nc1nccc2[nH]nc(-c3cc(C(=O)N(C)C)ccn3)c12] |
| 56 | (2-(4-(isopropylamino)-1H- pyrazolo[4,3-c]pyridin-3- yl)pyridin-4- yl)(morpholino)methanone | 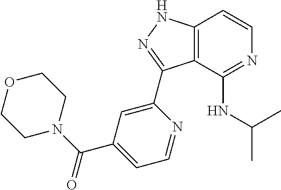 [SMILES: CC(C)Nc1nccc2[nH]nc(-c3cc(C(=O)N4CCOCC4)ccn3)c12] |
| 57 | 6-methyl-N-(tetrahydro-2H- pyran-4-yl)-3-(6- (trifluoromethyl)pyrimidin-4- yl)-1H-pyrazolo[4,3- c]pyridin-4-amine | 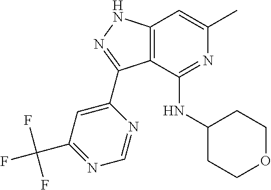 [SMILES: Cc1cc2[nH]nc(-c3cc(C(F)(F)F)ncn3)c2c(NC2CCOCC2)n1] |
| 58 | 3-(6-ethoxypyrimidin-4-yl)- 6-methyl-N-(tetrahydro-2H- pyran-4-yl)-1H-pyrazolo[4,3- c]pyridin-4-amine | 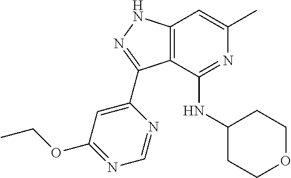 [SMILES: CCOc1cc(-c2n[nH]c3cc(C)nc(NC4CCOCC4)c23)ncn1] |
| 59 | 3-(4-((3R,4R)-3,4- difluoropyrrolidin-1- yl)pyridin-2-yl)-N-isopropyl- 1H-pyrazolo[4,3-c]pyridin-4- amine | 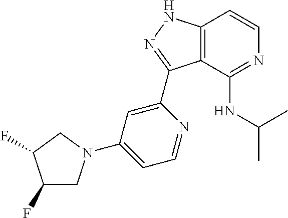 [SMILES: CC(C)Nc1nccc2[nH]nc(-c3cc(N4C[C@@H](F)[C@H](F)C4)ccn3)c12] |
| 60 | N,N-dimethyl-2-(6-methyl- 4-((tetrahydro-2H-pyran-4- yl)amino)-1H-pyrazolo[4,3- c]pyridin-3- yl)isonicotinamide | 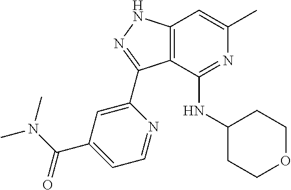 [SMILES: Cc1cc2[nH]nc(-c3cc(C(=O)N(C)C)ccn3)c2c(NC2CCOCC2)n1] |
| 61 | 3-(4-(2,2,6,6-tetrafluoro- morpholino)pyridin- 2-yl)-N-(tetrahydro-2H- pyran-4-yl)-1H-pyrazolo[4,3- c]pyridin-4-amine | 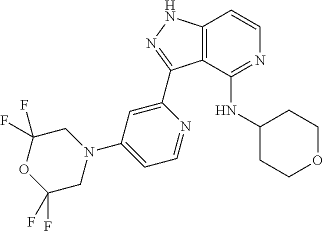 [SMILES: FC1(F)CN(c2ccnc(-c3n[nH]c4ccnc(NC5CCOCC5)c34)c2)CC(F)(F)O1] |
| 62 | 6-(4-(isopropylamino)-1H- pyrazolo[4,3-c]pyridin-3-yl)- N,N-dimethylpyrimidine-4- carboxamide | 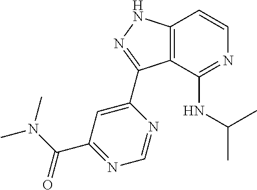 [SMILES: CC(C)Nc1nccc2[nH]nc(-c3cc(C(=O)N(C)C)ncn3)c12] |
| 63 | azetidin-1-yl(2-(4- ((tetrahydro-2H-pyran-4- yl)amino)-1H-pyrazolo[4,3- c]pyridin-3-yl)pyridin-4- yl)methanone | 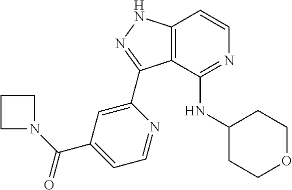 [SMILES: O=C(c1ccnc(-c2n[nH]c3ccnc(NC4CCOCC4)c23)c1)N1CCC1] |
| 64 | N,N,2-trimethyl-6-(4- ((tetrahydro-2H-pyran-4- yl)amino)-1H-pyrazolo[4,3- c]pyridin-3- yl)isonicotinamide | 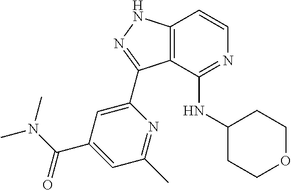 [SMILES: Cc1cc(C(=O)N(C)C)cc(-c2n[nH]c3ccnc(NC4CCOCC4)c23)n1] |
| 65 | N-(cyclopropylmethyl)-3-(6- (trifluoromethyl)pyrimidin-4- yl)-1H-pyrazolo[4,3- c]pyridin-4-amine | 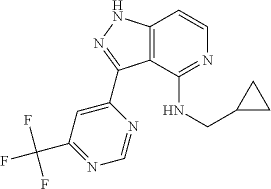 [SMILES: FC(F)(F)c1cc(-c2n[nH]c3ccnc(NCC4CC4)c23)ncn1] |
| 66 | N-(4,4-difluorocyclohexyl)- 3-(6-(trifluoro- methyl)pyrimidin-4- yl)-1H-pyrazolo[4,3- c]pyridin-4-amine | 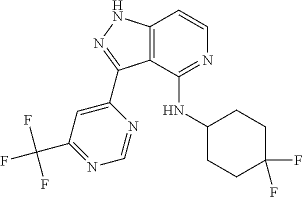 [SMILES: FC1(F)CCC(Nc2nccc3[nH]nc(-c4cc(C(F)(F)F)ncn4)c23)CC1] |
| 67 | N-(4,4-difluorocyclohexyl)- 3-(2-methyl-6- (trifluoromethyl)pyrimidin-4- yl)-1H-pyrazolo[4,3- c]pyridin-4-amine | 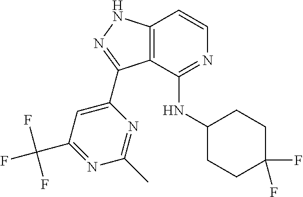 [SMILES: Cc1nc(-c2n[nH]c3ccnc(NC4CCC(F)(F)CC4)c23)cc(C(F)(F)F)n1] |
| 68 | N-(2,6-dimethyltetrahydro- 2H-pyran-4-yl)-3-(6- (trifluoromethyl)pyrimidin-4- yl)-1H-pyrazolo[4,3- c]pyridin-4-amine | 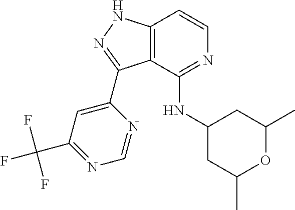 [SMILES: CC1CC(Nc2nccc3[nH]nc(-c4cc(C(F)(F)F)ncn4)c23)CC(C)O1] |
| 69 | 3-(4- (methylsulfonyl)pyridin-2- yl)-N-(tetrahydro-2H-pyran- 4-yl)-1H-pyrazolo[4,3- c]pyridin-4-amine | 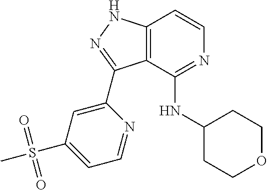 [SMILES: CS(=O)(=O)c1ccnc(-c2n[nH]c3ccnc(NC4CCOCC4)c23)c1] |
| 70 | 3-(6-methyl-4- (trifluoromethyl)pyridin-2- yl)-N-(tetrahydro-2H-pyran- 4-yl)-1H-pyrazolo[4,3- c]pyridin-4-amine | 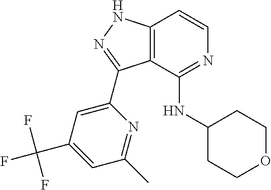 [SMILES: Cc1cc(C(F)(F)F)cc(-c2n[nH]c3ccnc(NC4CCOCC4)c23)n1] |
| 71 | 3-(2-methyl-6- (trifluoromethyl)pyrimidin-4- yl)-N-(tetrahydro-2H-pyran- 4-yl)-1H-pyrazolo[4,3- c]pyridin-4-amine | 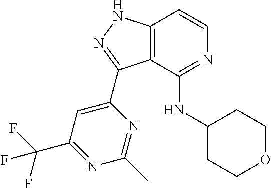 [SMILES: Cc1nc(-c2n[nH]c3ccnc(NC4CCOCC4)c23)cc(C(F)(F)F)n1] |
| 72 | N-isopropyl-3-(4- (methylsulfonyl)pyridin-2- yl)-1H-pyrazolo[4,3- c]pyridin-4-amine | 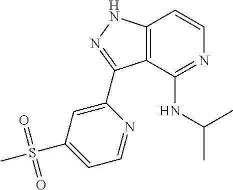 [SMILES: CC(C)Nc1nccc2[nH]nc(-c3cc(S(C)(=O)=O)ccn3)c12] |
| 73 | N-(dicyclopropylmethyl)- 3-(6-(trifluoro- methyl)pyrimidin-4- yl)-1H-pyrazolo[4,3- c]pyridin-4-amine | 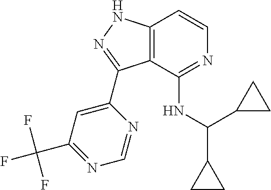 [SMILES: FC(F)(F)c1cc(-c2n[nH]c3ccnc(NC(C4CC4)C4CC4)c23)ncn1] |
| 74 | N-isopropyl-3-(4- (isopropylsulfonyl)pyridin-2- yl)-1H-pyrazolo[4,3- c]pyridin-4-amine | 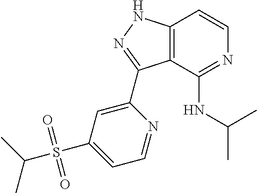 [SMILES: CC(C)Nc1nccc2[nH]nc(-c3cc(S(=O)(=O)C(C)C)ccn3)c12] |
| 75 | 3-(4- (isopropylsulfonyl)pyridin-2- yl)-N-(tetrahydro-2H-pyran- 4-yl)-1H-pyrazolo[4,3- c]pyridin-4-amine | 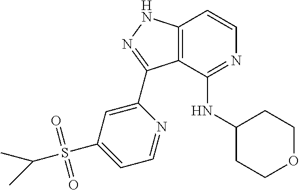 [SMILES: CC(C)S(=O)(=O)c1ccnc(-c2n[nH]c3ccnc(NC4CCOCC4)c23)c1] |
| 76 | N-isopropyl-3-(6-methyl-4- (methylsulfonyl)pyridin-2- yl)-1H-pyrazolo[4,3- c]pyridin-4-amine | 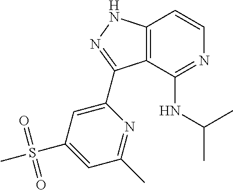 [SMILES: Cc1cc(S(C)(=O)=O)cc(-c2n[nH]c3ccnc(NC(C)C)c23)n1] |
| 77 | N-(8-oxabicyclo[3.2.1]octan- 3-yl)-3-(6- (trifluoromethyl)pyrimidin-4- yl)-1H-pyrazolo[4,3- c]pyridin-4-amine | 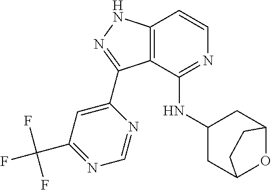 [SMILES: FC(F)(F)c1cc(-c2n[nH]c3ccnc(NC4CC5CCC(C4)O5)c23)ncn1] |

In one embodiment of the invention, the compound has a Ki value of less than about 0.1 μM for LRRK2, or less than about 0.01 μM, or less than about 0.001 μM.

Advantageously, the claimed compounds exhibit surprisingly good inhibition of LRRK2 (see Table 1), compared to compounds previously described in the art, for example, in WO 2010/106333 and WO 2011/141756. For comparative purposes, the structure and Ki values for various compounds described in WO 2010/106333 and WO 2011/141756 are shown in Table 2.

Therapeutic Applications

A further aspect of the invention relates to a compound as described above for use in medicine.

Another aspect of the invention relates to a compound as described above for use in treating cancer or a neurodegenerative disorder.

Another aspect relates to the use of a compound as described above in the preparation of a medicament for treating or preventing a neurodegenerative disorder. The neurodegenerative disorder is Parkinson's Disease.

Another aspect relates to the use of a compound as described above in the preparation of a medicament for treating or preventing a proliferative disorder, for example, cancer.

The compound is administered in an amount sufficient to inhibit one or more kinases such as LRRK2.

Yet another aspect relates to the use of a compound of the invention in the preparation of a medicament for the prevention or treatment of a disorder caused by, associated with or accompanied by any abnormal activity against a biological target, wherein the target is a kinase such as LRRK2.

The disorder may be Parkinson's Disease.

Another aspect of the invention relates to a method of treating a protein kinase related disease or disorder. The method according to this aspect of the present invention is effected by administering to a subject in need thereof a therapeutically effective amount of a compound of the present invention, as described hereinabove, either per se, or, as a part of a pharmaceutical composition, mixed with, for example, a pharmaceutically acceptable carrier, as is detailed hereinafter.

Yet another aspect of the invention relates to a method of treating a mammal having a disease state alleviated by inhibition of a protein kinase, wherein the method comprises administering to a mammal a therapeutically effective amount of a compound according to the invention.

The disease state may be alleviated by the inhibition of the protein kinase LRRK, more preferably LRRK2.

The mammal may be a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administering” as used herein refers to a method for bringing a compound of the present invention and a protein kinase together in such a manner that the compound can affect the enzyme activity of the protein kinase either directly; i.e., by interacting with the protein kinase itself or indirectly; i.e., by interacting with another molecule on which the catalytic activity of the protein kinase is dependent. As used herein, administration can be accomplished either in vitro, i.e. in a test tube, or in vivo, i.e., in cells or tissues of a living organism.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. The data obtained from these cell cultures assays and animal studies can be used in formulating a dosage range that is not toxic for use in human. The dosage of such compounds lies within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al, 1975, In: The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/kg/day, commonly from about 100-1000 mg/kg/day, or from about 150-700 mg/kg/day, or from about 250-500 mg/kg/day. Therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “kinase related disease or disorder” refers to a disease or disorder characterized by inappropriate kinase activity or over-activity of a kinase as defined herein. Inappropriate activity refers to either; (i) kinase expression in cells which normally do not express said kinase; (ii) increased kinase expression leading to unwanted cell proliferation, differentiation and/or growth; or, (iii) decreased kinase expression leading to unwanted reductions in cell proliferation, differentiation and/or growth. Over-activity of kinase refers to either amplification of the gene encoding a particular kinase or production of a level of kinase activity, which can correlate with a cell proliferation, differentiation and/or growth disorder (that is, as the level of the kinase increases, the severity of one or more of the symptoms of the cellular disorder increases). Over activity can also be the result of ligand independent or constitutive activation as a result of mutations such as deletions of a fragment of a kinase responsible for ligand binding.

Diseases or disorders that the compounds described herein may be useful in preventing, include cancer and neurodegenerative disorders such as Parkinson's Disease.

Thus, the present invention further provides use of compounds as defined herein for the manufacture of medicaments for the treatment of diseases where it is desirable to inhibit LRRK2. Such diseases include Parkinson's Disease.

Pharmaceutical Compostions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including antioxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M, or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (1) in conjunction or association with a pharmaceutically or veterinarily acceptable carrier or vehicle.

Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes may be used for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be used in some embodiments. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

Prodrugs

The invention further includes the compounds of the present invention in prodrug form, i.e. covalently bonded compounds which release the active parent drug according to general formula (I) in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Solvates

The present invention also includes solvate forms of the compounds of the present invention. The terms used in the claims encompass these forms.

Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. The formulation may be an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. These compositions contain from 1 to 250 mg, or from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, or between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of general formula (I) may be administered to inhibit the kinase implicated with a particular condition or disease. Of course, this dosage amount will further be modified according to the type of administration of the compound. For example, to achieve an “effective amount” for acute therapy, parenteral administration of a compound of general formula (I) may be used. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; or between 0.1 and 20 mg/kg, in a manner to maintain the concentration of drug in the plasma at a concentration effective to inhibit a kinase. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 50 mg/kg in a manner consistent with the condition of the patient. The oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

Combinations

In one embodiment, the one or more compounds of the invention are administered in combination with one or more other active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the inhibitory activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

Assay

A further aspect of the invention relates to the use of a compound as described above in an assay for identifying further candidate compounds capable of inhibiting one or more kinases, such as LRRK2.

The assay may be a competitive binding assay.

In certain embodiments the competitive binding assay comprises contacting a compound of the invention with a kinase, such as LRRK2, and a candidate compound and detecting any change in the interaction between the compound according to the invention and the kinase.

The candidate compound may be generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

The competitive binding assay may comprise contacting a compound of the invention with a kinase in the presence of a known substrate of said kinase and detecting any change in the interaction between said kinase and said known substrate.

A further aspect of the invention provides a method of detecting the binding of a ligand to a kinase, said method comprising the steps of:
(i) contacting a ligand with a kinase in the presence of a known substrate of said kinase;
(ii) detecting any change in the interaction between said kinase and said known substrate;

and wherein said ligand is a compound of the invention.

One aspect of the invention relates to a process comprising the steps of:

(a) performing an assay method described hereinabove;

(b) identifying one or more ligands capable of binding to a ligand binding domain; and

(c) preparing a quantity of said one or more ligands.

Another aspect of the invention provides a process comprising the steps of:

(a) performing an assay method described hereinabove;

(b) identifying one or more ligands capable of binding to a ligand binding domain; and

(c) preparing a pharmaceutical composition comprising said one or more ligands.

Another aspect of the invention provides a process comprising the steps of:

(a) performing an assay method described hereinabove;

(b) identifying one or more ligands capable of binding to a ligand binding domain;

(c) modifying said one or more ligands capable of binding to a ligand binding domain;

(d) performing the assay method described hereinabove;

(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of one or more disorders [insert list of disorders].

The above methods may be used to screen for a ligand useful as an inhibitor of one or more kinases.

Compounds of general formula (1) are useful both as laboratory tools and as therapeutic agents. In the laboratory certain compounds of the invention are useful in establishing whether a known or newly discovered kinase contributes a critical or at least significant biochemical function during the establishment or progression of a disease state, a process commonly referred to as ‘target validation’.

Synthesis

A further aspect of the invention relates to a process for preparing a compound of formula (1A) or (1B) as defined above, said process comprising the steps of:
(i) converting a compound of formula (4) into a compound of formula (3), where PG is a protecting group;
(ii) reacting said compound of formula (3) with a compound of formula R¹—NH₂ to form a compound of formula (2);
(iii) converting said compound of formula (2) to a compound of formula (1A) or (1B).

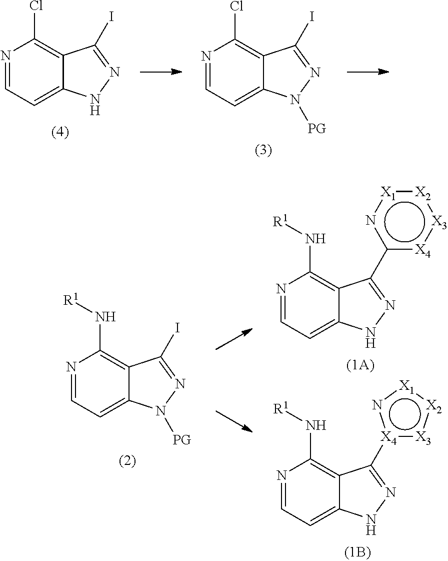

Suitable protecting groups, PG, will be familiar to a person skilled in the art (see, for example, Greene, Theodora W. and Wuts, Peter G. M. Greene's Protective Groups in Organic Synthesis. 4th Ed. (2006)), and include, but are not limited to, tert-butoxycarbonyl, benzyloxycarbonyl, benzyl, 4-methoxybenzyl, 2,4-dimethoxybenzyl or trityl. In one embodiment, the protecting group, PG, is trityl or 4-methoxybenzyl (PMB).

In one embodiment, step (iii) comprises reacting said compound of formula (2) with 1,1,1,2,2,2-hexamethyldistannane and trans-Pd(PPh₃)₂Cl₂ to form an intermediate of formula (2A):

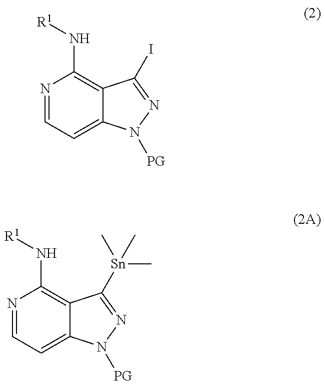

and converting said compound of formula (2A) into a compound of formula (1A) or (1B) by a suitable coupling reaction. The coupling may take place in the presence of a palladium catalyst, such as Pd(PPh₃)₄/CuI. The compound of formula (2A) may be coupled with a compound of formula (5A) or (5B),

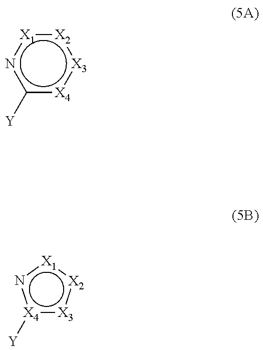

wherein Y is a halo (such as bromo) and X₁-X₄ are as defined for claim 1.

In one embodiment, the compound resulting from the above coupling of (2A) with (5A) or (5B) is further modified to form a compound of formula (1A) or (1B).

The invention is further described by way of the following non-limiting examples, and with reference to the following figures, wherein:

FIG. 1 shows the domain structure of LRRK1 and local mutations that have been linked to Parkinson's disease.

### EXAMPLES

### Materials and Methods

Source and Purification of Kinases

All LRRK2 protein kinases were of human origin and were sourced from Invitrogen Corporation (Carlsbad, Calif. 92008 USA) unless otherwise indicated. The active mutant used was recombinant human, catalytic domain (amino acids 970-2527) containing a G2019S mutation, GST-tagged, expressed in insect cells (Invitrogen Cat#PV4881). The wild type used was recombinant human, catalytic domain (amino acids 970-2527) GST-tagged, expressed in insect cells (Invitrogen Cat#PV4873). The kinase dead mutant used was recombinant human, catalytic domain (amino acids 970-2527) containing a D1994A mutation, GST-tagged, expressed in insect cells (Invitrogen Cat#PM4041AE). No special measures were taken to activate any of the kinases.

Protein Kinase Assays

All assays were carried out at room temperature (˜21° C.) and were linear with respect to time and enzyme concentration under the conditions used. Assays were performed for 180 min in a 96 well format. LRRK2 was present at a concentration of approximately 5 nM. The enzyme was diluted and assayed in 50 mM Tris-HCl pH7.5, 0.1 mM EGTA, 1 mM DTT and 10 mM MgCl₂. The concentration of magnesium chloride in the assay was 10 mM. The [γ-33P] ATP (0.4 μCi/well) was used at 134 uM for G2019S mutant and at 57 μM for the wild type kinase in order to be at Km. The peptide substrate in the assay was RLGWWRFYTLRRARQGNTKQR at 100 μM.

The assays were initiated with Mg/ATP and stopped by the addition of 25 μl/well 50% orthophosphoric acid. Reactions were harvested onto Whatman P81 Unifilter Plates (Fisher Scientific. Loughborough, LE115RG, UK. Cat# FDU-105-020U) using a Tomtec harvester. (Tomtec Hamden, Conn. 06514. USA) Plates were counted using a Perkin Elmer Top Count NX7. (Perkin Elmer, Shelton Conn. 06484-4794 USA)

IC50 values of inhibitors were determined after carrying out assays at 10 different concentrations of each compound in duplicate.

General Procedures for Synthesis of Compounds

Chromatography

Preparative high pressure liquid chromatography was carried out using apparatus made by Agilent. The apparatus is constructed such that the chromatography is monitored by a multi-wavelength UV detector (G1365B manufactured by Agilent) and an MM-ES+APCI mass spectrometer (G-1956A, manufactured by Agilent) connected in series, and if the appropriate criteria are met the sample is collected by an automated fraction collector (G1364B manufactured by Agilent). Collection can be triggered by any combination of UV or mass spectrometry or can be based on time. Typical conditions for the separation process are as follows: The gradient is run over a 10 minute period (gradient at start: 10% methanol and 90% water, gradient at finish: 100% methanol and 0% water; as buffer: either 0.1% trifluoroacetic acid is added to the water (low pH buffer), or ammonium bicarbonate (10 mmol/1) and 35% ammonium hydroxide (1.6 ml/1) is added to the water (high pH buffer). It will be appreciated by those skilled in the art that it may be necessary or desirable to modify the conditions for each specific compound, for example by changing the solvent composition at the start or at the end, modifying the solvents or buffers, changing the run time, changing the flow rate and/or the chromatography column.

Flash chromatography refers to silica gel chromatography and carried out using an SP4 or an Isolara 4 MPLC system (manufactured by Biotage); pre-packed silica gel cartridges (supplied by Biotage); or using conventional glass column chromatography.

Analytical Methods

¹H Nuclear magnetic resonance (NMR) spectroscopy was carried out using an ECX400 spectrometer (manufactured by JEOL) in the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; br, broad. Mass spectra were recorded using a MM-ES+APCI mass spectrometer (G-1956A, manufactured by Agilent). Where thin layer chromatography (TLC) has been used it refers to silica gel TLC using silica gel MK6F 60 Å plates, R_{f} is the distance travelled by the compound divided by the distance travelled by the solvent on a TLC plate.

Compound Preparation

Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is an Initiator 60 supplied by Biotage. The actual power supplied varies during the course of the reaction in order to maintain a constant temperature.

Abbreviations

DCM=Dichloromethane

DMF=N,N-Dimethylformamide

THF=Tetrahydrofuran

MeOH=Methanol

TFA=Trifluoroacetic acid

Xantphos=4,5-Bis(diphenylphosphino)-9,9-dimethylxanthene

HATU=N,N,N′,N′-Tetramethyl-O-(7-azabenzotriazol-1-yl)uronium-hexafluorophospate

EDCI=1,3-Propanediamine, N3-(ethylcarbonimidoyl)-N1,N1-dimethyl-, hydrochloride

DCC=1,3-Dicyclohexylcarbodiimide

Pd₂(dba)₃=tris(dibenzylideneacetone)dipalladium(0)

TEA=Triethylamine

rm=Reaction mixture

rt=Room temperature

AcOH=Acetic acid

IPA=Isopropanol

DIPEA=N,N-diisopropylethylamine

TBSMSCl=Tertiarybutyldimethylsilyl chloride

MeCN=Acetonitrile

NH₃=Ammonia

EtOH=Ethanol

EtOAc=Ethyl Acetate

LCMS=Mass spectrometry directed high pressure liquid chromatography

UV=Ultraviolet

SCX=Strong cation exchange

TPAP=Tetrapropylammonium perruthenate

DMSO=Dimethylsulphoxide

BINAP=2,2′-bis(diphenylphosphino)-1,1′-binaphthyl

### EXAMPLES

### N-isopropyl-3-(pyridin-2-yl)-1H-pyrazolo[4,3-c]pyridin-4-amine

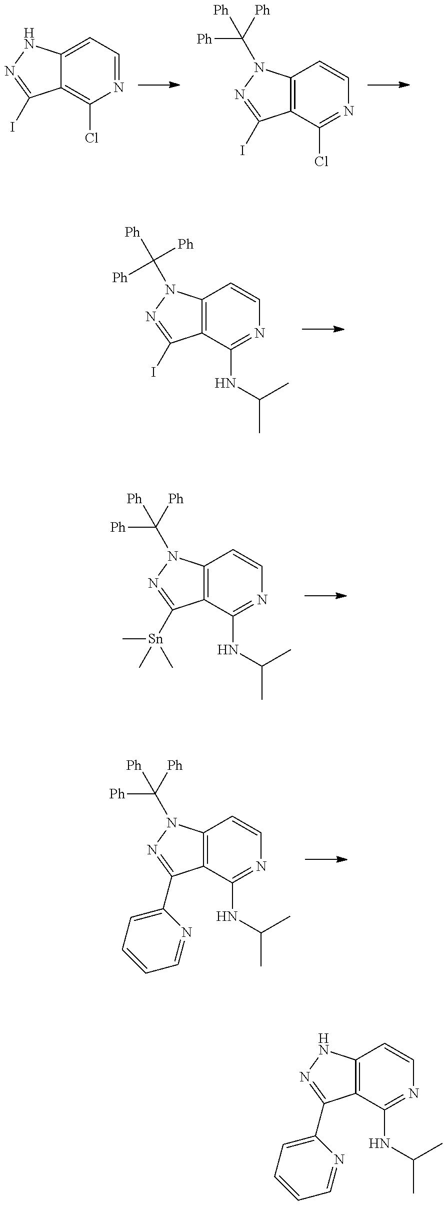 [SMILES: Clc1nccc2[nH]nc(I)c12 ; Clc1nccc2c1c(I)nn2C(c1ccccc1)(c1ccccc1)c1ccccc1 ; CC(C)Nc1nccc2c1c(I)nn2C(c1ccccc1)(c1ccccc1)c1ccccc1 ; CC(C)Nc1nccc2c1[c]([Sn]([CH3])([CH3])[CH3])nn2C(c1ccccc1)(c1ccccc1)c1ccccc1 ; CC(C)Nc1nccc2c1c(-c1ccccn1)nn2C(c1ccccc1)(c1ccccc1)c1ccccc1 ; CC(C)Nc1nccc2[nH]nc(-c3ccccn3)c12]

Step 1—Synthesis of 4-chloro-3-iodo-1-trityl-1H-pyrazolo[4,3-c]pyridine: A suspension of 4-chloro-3-iodo-1H-pyrozolo[4,3-c]pyridine (7.88 g, 0.0280 mol) in DCM (100 mL) was stirred at 0° C. for 5 minutes. TEA (5.62 g, 0.0560 mol) was added and the mixture was stirred for 10 minutes. Trityl chloride (11.7 g, 0.0420 mol) was then added. After being stirred for 3 hours, the reaction mixture was treated with water (100 mL). The organic layer was washed with brine, dried over anhydrous sodium sulfate, and concentrated to give the title compound as an off-white solid (13.2 g, 89%).

Step 2—Synthesis of 3-iodo-N-isopropyl-1-trityl-1H-pyrazolo[4,3-c]pyridin-4-amine: A microwave tube equipped with a magnetic stirrer was charged with 4-chloro-3-iodo-1-trityl-1H-pyrazolo[4,3-c]pyridine (2.00 g, 3.84 mmol), propan-2-amine (1.10 g, 19.2 mmol), n-BuOH (0.86 g, 7.68 mmol), and DIPEA (10 mL). The reaction mixture was heated at 170° C. for 2 h under microwave irradiation. After cooling down, the mixture was concentrated under reduced pressure. The residue was purified by silica gel column chromatography eluting with DCM/petroleum ether/TEA (2/1/0.01) to afford the title compound as a white solid (1.4 g, 67%).

Step 3—Synthesis of N-isopropyl-3-(trimethylstannyl)-1-trityl-1H-pyrazolo[4,3-c]pyridin-4-amine: A reaction tube equipped with a magnetic stirrer was charged with 3-iodo-N-isopropyl-1-trityl-1H-pyrazolo[4,3-c]pyridin-4-amine (1.00 g, 1.84 mmol), 1,1,1,2,2,2-hexamethyldistannane (723 mg, 2.21 mmol), toluene (12 mL), and trans-Pd(PPh₃)₂Cl₂ (40 mg, 0.055 mmol). After three cycles of vacuum/argon flash, the tube was sealed and heated at 140° C. for 0.5 h. After cooling down to room temperature, the mixture was filtered via Celite. The filtrate was concentrated under reduced pressure to afford the crude title compound, which was used in next step directly without further purification.

Step 4—Synthesis of N-isopropyl-3-(pyridin-2-yl)-1-trityl-1H-pyrazolo[4,3-c]pyridin-4-amine: A microwave vial equipped with a magnetic stirrer was charged with N-isopropyl-3-(trimethylstannyl)-1-trityl-1H-pyrazolo[4,3-c]pyridin-4-amine (100 mg, 0.172 mmol), 2-bromopyridine (54.0 mg, 0.344 mmol), LiCl (29.0 mg, 0.688 mmol), CuI (7.0 mg, 0.034 mmol), Pd(PPh₃)₄ (20.0 mg, 0.0172 mmol) and THF (2 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1/2) to afford the title compound as a yellow solid (65 mg, 76%).

Step 5—Synthesis of N-isopropyl-3-(pyridin-2-yl)-1H-pyrazolo[4,3-c]pyridin-4-amine: To a mixture of N-isopropyl-3-(pyridin-2-yl)-1-trityl-1H-pyrazolo[4,3-c]pyridin-4-amine (65.0 mg, 0.131 mmol) in dichloromethane (5 mL) was added trifluoroacetic acid (1.0 mL) and triethylsilane (0.20 mL). The mixture was stirred under reflux for 1 hour. After cooling down, the reaction mixture was neutralized to pH around 7 with saturated NaHCO₃ solution. The resulting mixture was extracted with ethyl acetate (30 mL×3). The organic layer was combined, dried, and concentrated in vacuo. The residue was purified by reverse phase prep-HPLC to give the title compound as a white solid (18 mg, 54%). ¹H NMR (500 MHz, CD₃OD) δ 8.63 (d, J=4.0 Hz, 1H), 8.38 (d, J=8.0 Hz, 1H), 7.96-7.92 (m, 1H), 7.68 (d, J=6.5 Hz, 1H), 7.44-7.41 (m, 1H), 6.64 (d, J=6.0 Hz, 1H), 4.23 (t, J=6.5 Hz, 1H), 1.39 (d, J=6.0 Hz, 6H).

### N-isopropyl-3-(4-morpholinopyridin-2-yl)-1H-pyrazolo[4,3-c]pyridin-4-amine

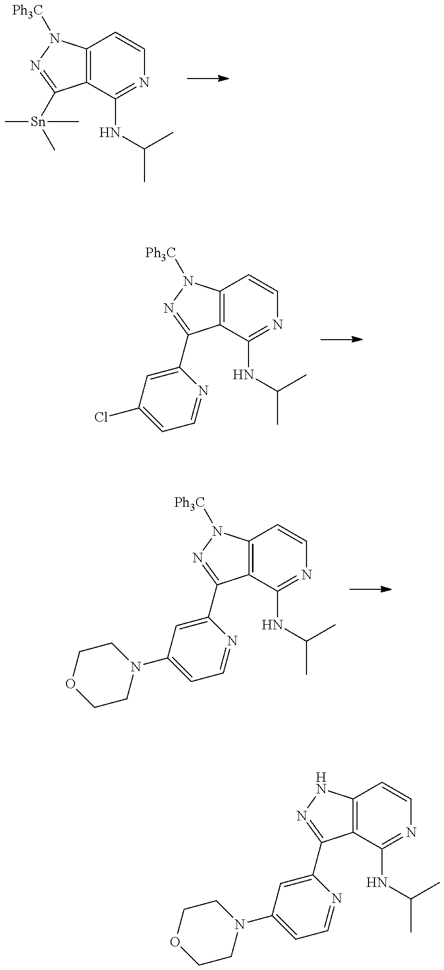

Step 1—Synthesis of 3-(4-chloropyridin-2-yl)-N-isopropyl-1-trityl-1H-pyrazolo[4,3-c]pyridin-4-amine: To a microwave tube was added N-isopropyl-3-(trimethylstannyl)-1-trityl-1H-pyrazolo[4,3-c]pyridin-4-amine (150 mg, 0.258 mmol), 2-bromo-4-chloropyridine (99.0 mg, 0.515 mmol), LiCl (43.0 mg, 1.03 mmol), CuI (10.0 mg, 0.0515 mmol), Pd(PPh₃)₄ (30.0 mg, 0.0258 mmol) and THF (3.0 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (100 mg, 73%).

Step 2—Synthesis of N-isopropyl-3-(4-morpholinopyridin-2-yl)-1-trityl-1H-pyrazolo[4,3-c]pyridin-4-amine: A mixture of 3-(4-chloropyridin-2-yl)-N-isopropyl-1-trityl-1H-pyrazolo[4,3-c]pyridin-4-amine (40.0 mg, 0.0756 mmol), morpholine (13.0 mg, 0.151 mmol), Pd₂(dba)₃ (7.0 mg, 0.0076 mmol), Xantphos (9.0 mg, 0.015 mmol), and Cs₂CO₃ (49.0 mg, 0.151 mmol) in dioxane (1.0 mL) in a microwave tube was stirred at 160° C. for 120 min under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (13 mg, 29%).

Step 3—Synthesis of N-isopropyl-3-(4-morpholinopyridin-2-yl)-1H-pyrazolo[4,3-c]pyridin-4-amine: To a mixture of N-isopropyl-3-(4-morpholinopyridin-2-yl)-1-trityl-1H-pyrazolo[4,3-c]pyridin-4-amine (13.0 mg, 0.0224 mmol) in dichloromethane (5.0 mL) was added trifluoroacetic acid (1.0 mL) and triethylsilane (0.20 mL). The mixture was stirred under reflux for 1 hour. After cooling down, the reaction mixture was neutralized to pH around 7 with saturated NaHCO₃ solution. The resulting mixture was extracted with ethyl acetate (30 mL×3). The organic layer was combined, dried, and concentrated in vacuo. The residue was purified by reverse phase prep-HPLC to give the title compound as a white solid (4.0 mg, 53%). ¹H NMR (500 MHz, CDCl₃) δ 12.22 (br s, 1H), 8.13 (d, J=6.0 Hz, 1H), 7.78 (d, J=2.5 Hz, 1H), 7.67 (d, J=5.0 Hz, 1H), 6.67 (dd, J=6.0, 2.0 Hz, 2H), 4.40-4.44 (m, 1H), 3.87 (t, J=5.0 Hz, 4H), 3.42 (t, J=5.0 Hz, 4H), 1.41 (d, J=6.0 Hz, 6H).

### N-isopropyl-3-(6-morpholinopyrimidin-4-yl)-1H-pyrazolo[4,3-c]pyridin-4-amine

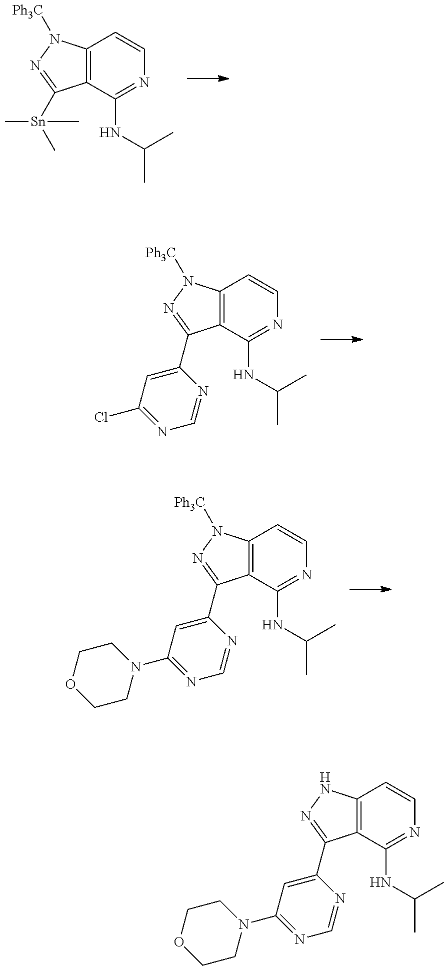

Step 1—Synthesis of 3-(6-chloropyrimidin-4-yl)-N-isopropyl-1-trityl-1H-pyrazolo[4,3-c]pyridin-4-amine: To a microwave tube was added N-isopropyl-3-(trimethylstannyl)-1-trityl-1H-pyrazolo[4,3-c]pyridin-4-amine (200 mg, 0.344 mmol), 4,6-dichloropyrimidine (102 mg, 0.688 mmol), LiCl (58.0 mg, 1.38 mmol), CuI (13.0 mg, 0.0688 mmol), Pd(PPh₃)₄ (40.0 mg, 0.0344 mmol) and THF (3.0 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (80 mg, 44%).

Step 2—Synthesis of N-isopropyl-3-(6-morpholinopyrimidin-4-yl)-1-trityl-1H-pyrazolo[4,3-c]pyridin-4-amine: A mixture of 3-(6-chloropyrimidin-4-yl)-N-isopropyl-1-trityl-1H-pyrazolo[4,3-c]pyridin-4-amine (80.0 mg, 0.151 mmol), morpholine (26.0 mg, 0.302 mmol), and Cs₂CO₃ (98.0 mg, 0.302 mmol) in i-PrOH (10 mL) was refluxed at 120° C. for 2 h. After filtration, the filtrate was evaporated to afford the title compound as a yellow solid (80 mg, 92%).

Step 3—Synthesis of N-isopropyl-3-(6-morpholinopyrimidin-4-yl)-1H-pyrazolo[4,3-c]pyridin-4-amine: To a mixture of N-isopropyl-3-(6-morpholinopyrimidin-4-yl)-1-trityl-1H-pyrazolo[4,3-c]pyridin-4-amine (80.0 mg, 0.138 mmol) in dichloromethane (5.0 mL) was added trifluoroacetic acid (1.0 mL) and triethylsilane (0.20 mL). The mixture was stirred under reflux for 1 hour. After cooling down, the reaction mixture was neutralized to pH around 7 with saturated NaHCO₃ solution. The mixture was extracted with ethyl acetate (30 mL×3). The organic layer was combined, dried, and concentrated in vacuo. The residue was purified by reverse phase prep-HPLC to give the title compound as a white solid of formic acid salt (20 mg, 38%). ¹H NMR (500 MHz, DMSO) δ 13.42 (br s, 1H), 10.39 (d, J=6.0 Hz, 1H), 8.68 (d, J=1.0 Hz, 1H), 8.15 (s, 1H), 7.74 (d, J=6.0 Hz, 1H), 7.51 (d, J=1.0 Hz, 1H), 6.60 (d, J=6.0 Hz, 1H), 4.19-4.26 (m, 1H), 3.70 (s, 8H), 1.28 (d, J=6.5 Hz, 6H).

### N-isopropyl-3-(4-morpholinopyrimidin-2-yl)-1H-pyrazolo[4,3-c]pyridin-4-amine

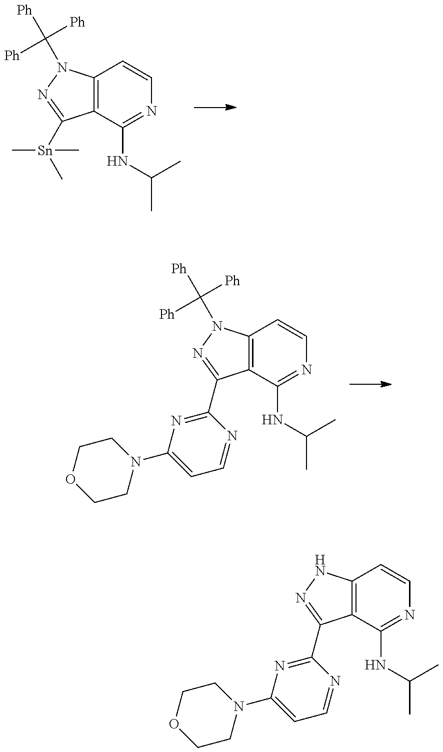 [SMILES: CC(C)Nc1nccc2c1[c]([Sn]([CH3])([CH3])[CH3])nn2C(c1ccccc1)(c1ccccc1)c1ccccc1 ; CC(C)Nc1nccc2c1c(-c1nccc(N3CCOCC3)n1)nn2C(c1ccccc1)(c1ccccc1)c1ccccc1 ; CC(C)Nc1nccc2[nH]nc(-c3nccc(N4CCOCC4)n3)c12]

Step 1—Synthesis of N-isopropyl-3-(4-morpholinopyrimidin-2-yl)-1-trityl-1H-pyrazolo[4,3-c]pyridin-4-amine: To a microwave tube was added N-isopropyl-3-(trimethylstannyl)-1-trityl-1H-pyrazolo[4,3-c]pyridin-4-amine (200 mg, 0.344 mmol), 4-(2-bromopyrimidin-4-yl)morpholine (168 mg, 0.688 mmol), LiCl (58.0 mg, 1.38 mmol), CuI (13.0 mg, 0.0688 mmol), Pd(PPh₃)₄ (40.0 mg, 0.0344 mmol), and THF (3.0 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (25 mg, 13%).

Step 2—Synthesis of N-isopropyl-3-(4-morpholinopyrimidin-2-yl)-1H-pyrazolo[4,3-c]pyridin-4-amine: To a mixture of N-isopropyl-3-(4-morpholinopyrimidin-2-yl)-1-trityl-1H-pyrazolo[4,3-c]pyridin-4-amine (25 mg, 0.043 mmol) in dichloromethane (5.0 mL) was added trifluoroacetic acid (1.0 mL) and triethylsilane (0.20 mL). The mixture was stirred under reflux for 1 hour. After cooling down, the reaction mixture was neutralized to pH around 7 with saturated NaHCO₃ solution. The resulting mixture was extracted with ethyl acetate (30 mL×3). The organic layer was combined, dried, and concentrated in vacuo. The residue was purified by reverse phase prep-HPLC to give the title compound as a white solid (3.0 mg, 20%). ¹H NMR (500 MHz, CDCl₃) δ 10.47 (br s, 1H), 8.27 (d, J=6.0 Hz, 1H), 7.87 (d, J=6.5 Hz, 1H), 6.60 (d, J=5.5 Hz, 1H), 6.52 (d, J=6.5 Hz, 1H), 4.39 (s, 1H), 3.76-3.83 (m, 8H), 1.37 (d, J=6.0 Hz, 6H).

### N-isopropyl-3-(pyrimidin-4-yl)-1H-pyrazolo[4,3-c]pyridin-4-amine

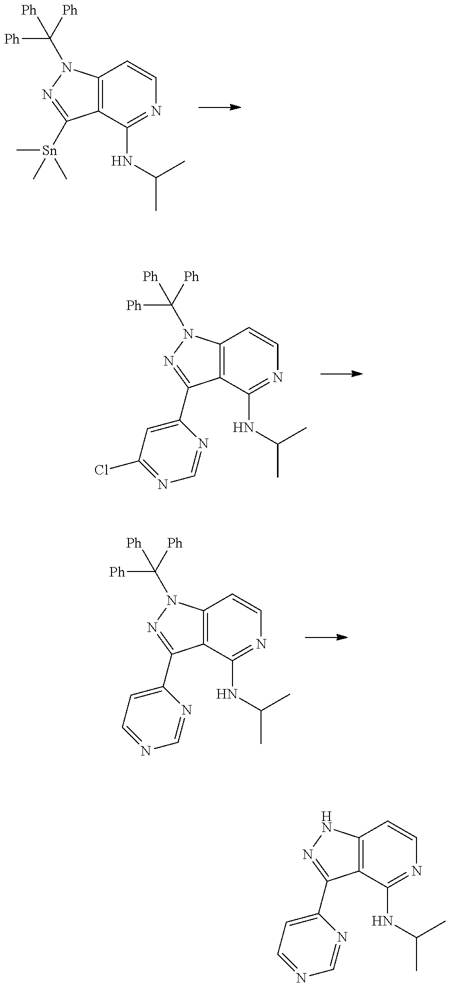 [SMILES: CC(C)Nc1nccc2c1[c]([Sn]([CH3])([CH3])[CH3])nn2C(c1ccccc1)(c1ccccc1)c1ccccc1 ; CC(C)Nc1nccc2c1c(-c1cc(Cl)ncn1)nn2C(c1ccccc1)(c1ccccc1)c1ccccc1 ; CC(C)Nc1nccc2c1c(-c1ccncn1)nn2C(c1ccccc1)(c1ccccc1)c1ccccc1 ; CC(C)Nc1nccc2[nH]nc(-c3ccncn3)c12]

Step 1—Synthesis of 3-(6-chloropyrimidin-4-yl)-N-isopropyl-1-trityl-1H-pyrazolo[4,3-c]pyridine-4-amine: A microwave vial equipped with a magnetic stirrer was charged with N-isopropyl-3-(trimethylstannyl)-1-trityl-1H-pyrazolo[4,3-c]pyridin-4-amine (100 mg, 0.172 mmol), 4,6-dichloropyrimidine (51.0 mg, 0.344 mmol), LiCl (29.0 mg, 0.688 mmol), CuI (7.0 mg, 0.034 mmol), Pd(PPh₃)₄ (20.0 mg, 0.0172 mmol), and THF (2 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1/2) to afford the title compound as a yellow solid (72 mg, 79%).

Step 2—Synthesis of N-isopropyl-3-(pyrimidin-4-yl)-1-trityl-1H-pyrazolo[4,3-c]pyridin-4-amine: To a mixture of 3-(6-chloropyrimidin-4-yl)-N-isopropyl-1-trityl-1H-pyrazolo[4,3-c]pyridine-4-amine (72.0 mg, 0.136 mmol) and Pd/C (10 mg, 10% w/w) in methanol (10 ml) was added a drop of NH₄OH. The mixture was stirred at 55° C. under H₂ overnight. After cooling down to room temperature, the mixture was filtered via Celite. The filtrate was concentrated under reduced pressure to afford the title compound as a yellow solid (65 mg, 97%).

Step 3—Synthesis of N-isopropyl-3-(pyrimidin-4-yl)-1H-pyrazolo[4,3-c]pyridin-4-amine: To a mixture of N-isopropyl-3-(pyrimidin-4-yl)-1-trityl-1H-pyrazolo[4,3-c]pyridin-4-amine (65.0 mg, 0.131 mmol) in dichloromethane (5 mL) was added trifluoroacetic acid (1.0 mL) and triethylsilane (0.20 mL). The mixture was stirred under reflux for 1 hour. After cooling down, the reaction mixture was neutralized to pH around 7 with saturated NaHCO₃ solution. The mixture was extracted with ethyl acetate (30 mL×3). The organic layer was combined, dried, and concentrated in vacuo. The residue was purified by reverse phase prep-HPLC to give the title compound as a yellow solid (20 mg, 60%). ¹H NMR (500 MHz, CD₃OD) δ 9.28 (d, J=1.0 Hz, 1H), 8.92 (d, J=5.5 Hz, 1H), 8.44 (dd, J=5.5, 1.5 Hz, 1H), 7.67 (J=6.5 Hz, 1H), 6.86 (d, J=6.5 Hz, 1H), 4.24-4.16 (m, 1H), 1.47 (d, J=6.5 Hz, 6H).

### N-ethyl-3-(pyridin-2-yl)-1H-pyrazolo[4,3-c]pyridin-4-amine

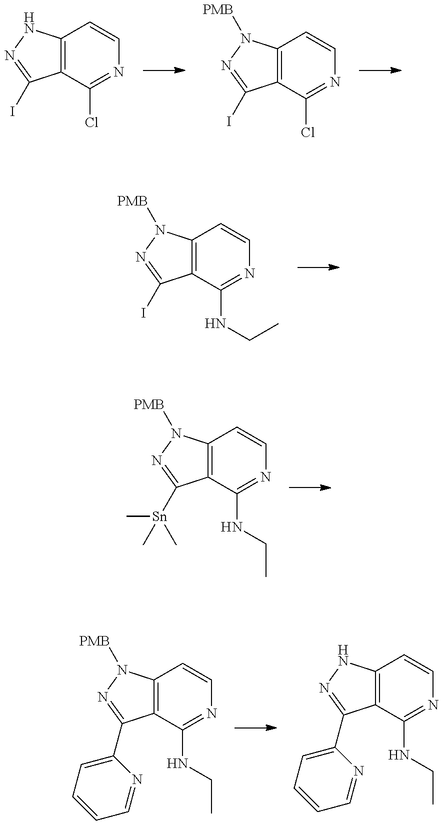

Step 1—Synthesis of 4-chloro-3-iodo-1-(4-methoxybenzyl)-1H-pyrazolo[4,3-c]pyridine: To a solution of 4-chloro-3-iodo-1H-pyrazolo[4,3-c]pyridine (2.00 g, 7.17 mmol) in DMF (30 mL) was added KOH (0.800 g, 14.3 mmol) and 1-(chloromethyl)-4-methoxybenzene (2.24 g, 14.3 mmol). The mixture was stirred at room temperature overnight. After concentration, the residue was purified by silica gel column chromatography eluting with petroleum ether/ethyl acetate (10:1 to 8:1) to give the title compound as a white solid (2.4 g, 82%).

Step 2—Synthesis of N-ethyl-3-iodo-1-(4-methoxybenzyl)-1H-pyrazolo[4,3-c]pyridin-4-amine: A microwave vial equipped with a magnetic stirrer was charged with 4-chloro-3-iodo-1-(4-methoxybenzyl)-1H-pyrazolo[4,3-c]pyridine (2.00 g, 3.84 mmol), ethanamine (865 mg, 19.2 mmol), and methanol (10 mL). The reaction mixture was heated at 150° C. for 2 h under microwave irradiation. After cooling down, the mixture was concentrated under reduced pressure. The residue was purified by silica gel column chromatography eluting with DCM/petroleum ether/TEA (2/1/0.01) to afford the title compound as a white solid (1.5 g, 73%).

Step 3—Synthesis of N-ethyl-1-(4-methoxybenzyl)-3-(trimethylstannyl)-1H-pyrazolo[4,3-c]-pyridin-4-amine: A reaction tube equipped with a magnetic stirrer was charged with N-ethyl-3-iodo-1-(4-methoxybenzyl)-1H-pyrazolo[4,3-c]pyridin-4-amine (750 mg, 1.84 mmol), 1,1,1,2,2,2-hexamethyldistannane (721 mg, 2.21 mmol), toluene (12 mL), and trans-Pd(PPh₃)₂Cl₂ (39.0 mg, 0.0551 mmol). After three cycles of vacuum/argon flash, the reaction mixture was sealed and heated at 140° C. for 0.5 h. After cooling down to room temperature, the mixture was filtered via Celite. The filtrate was concentrated under reduced pressure to afford the crude title compound, which was used in next step directly without further purification.

Step 4—Synthesis of N-ethyl-1-(4-methoxybenzyl)-3-(pyridin-2-yl)-1H-pyrazolo[4,3-c]pyridin-4-amine: A microwave vial equipped with a magnetic stirrer was charged with N-ethyl-1-(4-methoxybenzyl)-3-(trimethylstannyl)-1H-pyrazolo[4,3-c]pyridin-4-amine (100 mg, 0.225 mmol), 2-bromopyridine (71 mg, 0.45 mmol), LiCl (38 mg, 0.9 mmol), CuI (9.0 mg, 0.045 mmol), Pd(PPh₃)₄ (26.0 mg, 0.0225 mmol), and THF (3 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (74 mg, 91%).

Step 5—Synthesis of N-ethyl-3-(pyridin-2-yl)-1H-pyrazolo[4,3-c]pyridin-4-amine: A microwave vial equipped with a magnetic stirrer was charged with N-ethyl-1-(4-methoxybenzyl)-3-(pyridin-2-yl)-1H-pyrazolo[4,3-c]pyridin-4-amine (74.0 mg, 0.206 mmol) and TFA (3.0 mL). The reaction mixture was heated at 120° C. for 2 h under microwave irradiation. After cooling down to room temperature, the mixture was concentrated under reduced pressure. The residue was purified by prep-HPLC to afford the title compound as a yellow solid (20 mg, 41%). ¹H NMR (500 MHz, CD₃OD) δ 8.65 (d, J=4.5 Hz, 1H), 8.42 (d, J=8.0 Hz, 1H), 8.00-7.97 (m, 1H), 7.64 (d, J=6.0 Hz, 1H), 7.48-7.46 (m, 1H), 6.77 (d, J=7.0 Hz, 1H), 3.58-3.53 (m, 2H), 1.74 (t, J=8.0 Hz, 3H).

### N-ethyl-3-(6-(trifluoromethyl)pyrimidin-4-yl)-1H-pyrazolo-[4,3-c]pyridin-4-amine

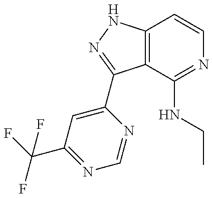 [SMILES: CCNc1nccc2[nH]nc(-c3cc(C(F)(F)F)ncn3)c12]

Synthesis of N-ethyl-3-(6-(trifluoromethyl)pyrimidin-4-yl)-1H-pyrazolo[4,3-c]pyridin-4-amine: The compound was prepared according to the general procedure described in the synthesis of N-ethyl-3-(pyridin-2-yl)-1H-pyrazolo[4,3-c]pyridin-4-amine by reacting N-ethyl-1-(4-methoxybenzyl)-3-(trimethylstannyl)-1H-pyrazolo[4,3-c]pyridin-4-amine with 4-bromo-6-(trifluoromethyl)pyrimidine to give the title compound as a yellow solid (22 mg, 26% yield over two steps). ¹H NMR (500 MHz, DMSO) δ 13.03 (br, 1H), 9.56 (s, 1H), 9.51 (s, 1H), 8.57 (d, J=1.0 Hz, 1H), 7.81 (d, J=6.0 Hz, 1H), 6.70 (d, J=6.0 Hz, 1H), 3.57-3.51 (m, 2H), 1.32 (t, J=7.5 Hz, 3H).

### 2-(4-(ethylamino)-1H-pyrazolo[4,3-c]pyridin-3-yl)isonicotinonitrile

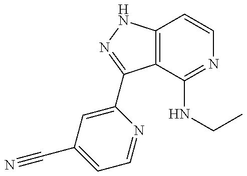 [SMILES: CCNc1nccc2[nH]nc(-c3cc(C#N)ccn3)c12]

Synthesis of 2-(4-(ethylamino)-1H-pyrazolo[4,3-c]pyridin-3-yl)isonicotinonitrile: The compound was prepared according to the general procedure described in the synthesis of N-ethyl-3-(pyridin-2-yl)-1H-pyrazolo[4,3-c]pyridin-4-amine by reacting N-ethyl-1-(4-methoxybenzyl)-3-(trimethylstannyl)-1H-pyrazolo[4,3-c]pyridin-4-amine with 2-bromoisonicotinonitrile to give the title compound as a yellow solid (7.0 mg, 12% yield over two steps). ¹H NMR (500 MHz, CD₃OD) δ 8.84-8.83 (m, 1H), 8.66 (s, 1H), 7.71-7.70 (m, 2H), 6.69 (d, J=6.5 Hz, 1H), 3.55-3.51 (m, 2H), 1.43 (t, J=7.5 Hz, 3H).

### N-ethyl-3-(4-(trifluoromethyl)pyridin-2-yl)-1H-pyrazolo[4,3-c]pyridin-4-amine

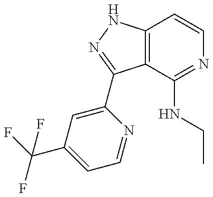 [SMILES: CCNc1nccc2[nH]nc(-c3cc(C(F)(F)F)ccn3)c12]

Synthesis of N-ethyl-3-(4-(trifluoromethyl)pyridin-2-yl)-1H-pyrazolo[4,3-c]pyridin-4-amine: The compound was prepared according to the general procedure described in the synthesis of N-ethyl-3-(pyridin-2-yl)-1H-pyrazolo[4,3-c]pyridin-4-amine by reacting N-ethyl-1-(4-methoxybenzyl)-3-(trimethylstannyl)-1H-pyrazolo[4,3-c]pyridin-4-amine with 2-bromo-4-(trifluoromethyl)pyridine to give the title compound as a yellow solid (10 mg, 15% yield over two steps). ¹H NMR (500 MHz, CD₃OD) δ 8.89 (d, J=5.5 Hz, 1H), 8.66 (s, 1H), 7.73-7.69 (m, 2H), 6.74 (d, J=6.0 Hz, 1H), 3.58-3.54 (m, 2H), 1.46 (t, J=7.5 Hz, 3H).

### N-ethyl-3-(pyrimidin-4-yl)-1H-pyrazolo[4,3-c]pyridin-4-amine

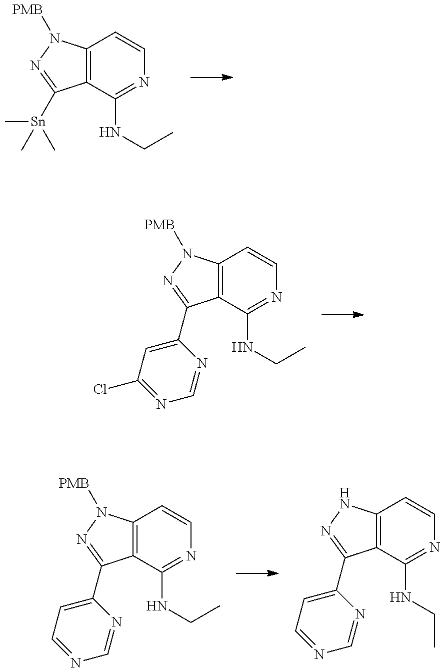

Step 1—Synthesis 3-(6-chloropyrimidin-4-yl)-N-ethyl-1-(4-methoxybenzyl)-1H-pyrazolo-[4,3-c]pyridin-4-amine: A microwave vial equipped with a magnetic stirrer was charged with N-ethyl-1-(4-methoxybenzyl)-3-(trimethylstannyl)-1H-pyrazolo[4,3-c]pyridin-4-amine (100 mg, 0.225 mmol), 4,6-dichloropyrimidine (67 mg, 0.45 mmol), LiCl (38 mg, 0.90 mmol), CuI (9.0 mg, 0.045 mmol), Pd(PPh₃)₄ (26.0 mg, 0.0225 mmol), and THF (3 mL). After three cycles of vacuum/argon flash, the reaction mixture was heated at 100° C. for 1 hour under microwave irradiation. After cooling down to room temperature, the mixture was filtered via Celite. The filtrate was concentrated under reduced pressure. The residue was purified by prep-TLC eluting with petroleum ether/ethyl acetate (1:2) to afford the title compound as a yellow solid (78 mg, 87%).

Step 2—Synthesis of N-ethyl-1-(4-methoxybenzyl)-3-(pyrimidin-4-yl)-1H-pyrazolo[4,3-c]-pyridin-4-amine: To a mixture of 3-(6-chloropyrimidin-4-yl)-N-isopropyl-1-trityl-1H-pyrazolo[4,3-c]-pyridine-4-amine (78.0 mg, 0.198 mmol) and Pd/C (10 mg, 10% w/w) in methanol (10 ml) was added a drop of NH₄OH. The mixture was stirred at 55° C. under H₂ overnight. After cooling down, the reaction mixture was flittered via Celite. The filtrate was concentrated to give the title compound as a yellow solid (65 mg, 91%).

Step 3—Synthesis of N-ethyl-3-(pyrimidin-4-yl)-1H-pyrazolo[4,3-c]pyridin-4-amine: A microwave vial equipped with a magnetic stirrer was charged with N-ethyl-1-(4-methoxybenzyl)-3-(pyrimidin-4-yl)-1H-pyrazolo[4,3-c]pyridin-4-amine (65 mg, 0.18 mmol) and TFA (3 mL). The reaction mixture was heated at 120° C. for 2 h under microwave irradiation. After cooling down to room temperature, the mixture was concentrated under reduced pressure. The residue was purified by prep-HPLC to give the title compound as yellow solid (18 mg, 41%). ¹H NMR (500 MHz, CD₃OD) δ 9.27 (d, J=1.0 Hz, 1H), 8.91 (d, J=6.0 Hz, 1H), 8.41 (dd, J=5.5, 1.5 Hz, 1H), 7.64 (d, J=7.0 Hz, 1H), 6.89 (d, J=7.0 Hz, 1H), 3.61-3.56 (m, 2H), 1.50 (t, J=7.0 Hz, 3H).

### N-(tetrahydro-2H-pyran-4-yl)-3-(4-(trifluoromethyl)pyridin-2-yl)-1H-pyrazolo[4,3-c]pyridin-4-amine

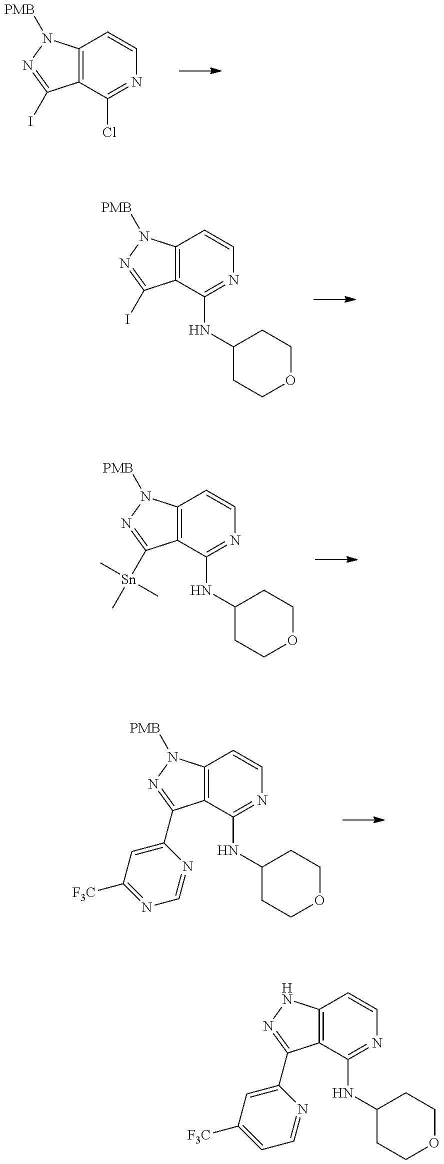

Step 1—Synthesis of 3-iodo-1-(4-methoxybenzyl)-N-(tetrahydro-2H-pyran-4-yl)-1H-pyrazolo[4,3-c]pyridin-4-amine: A microwave vial equipped with a magnetic stirrer was charged with 4-chloro-3-iodo-1-(4-methoxybenzyl)-1H-pyrazolo[4,3-c]pyridine (2.60 g, 6.50 mmol), tetrahydro-2H-pyran-4-amine (1.97 g, 19.5 mmol), n-BuOH (15 mL), and DIPEA (2.53 g, 19.5 mmol). The reaction mixture was heated at 180° C. for 4 h under microwave irradiation. After cooling down, the solid was collected and washed with EtOH to afford the title compound as a white solid (2.35 g, 78%).

Step 2—Synthesis of 1-(4-methoxybenzyl)-N-(tetrahydro-2H-pyran-4-yl)-3-(trimethylstannyl)-1H-pyrazolo[4,3-c]pyridin-4-amine: A reaction tube equipped with a magnetic stirrer was charged with 3-iodo-1-(4-methoxybenzyl)-N-(tetrahydro-2H-pyran-4-yl)-1H-pyrazolo[4,3-c]pyridin-4-amine (600 mg, 1.29 mmol), 1,1,1,2,2,2-hexamethyldistannane (507 mg, 1.55 mmol), toluene (12 mL), and trans-Pd(PPh₃)₂Cl₂ (24.0 mg, 0.0314 mol). After three cycles of vacuum/argon flash, the reaction mixture was heated at 140° C. for 1 h. After cooling down, the reaction mixture was filtered. The filtrate was concentrated to give the title compound as a yellow solid which was used for the next step without further purification (850 mg).

Step 3—Synthesis of 1-(4-methoxybenzyl)-N-(tetrahydro-2H-pyran-4-yl)-3-(6-(trifluoromethyl)pyrimidin-4-yl)-1H-pyrazolo[4,3-c]pyridin-4-amine: A microwave vial equipped with a magnetic stirrer was charged with 1-(4-methoxybenzyl)-N-(tetrahydro-2H-pyran-4-yl)-3-(trimethylstannyl)-1H-pyrazolo[4,3-c]pyridin-4-amine (500 mg, 0.997 mmol), 4-bromo-6-(trifluoromethyl)pyrimidine (271 mg, 1.19 mmol), LiCl (184 mg, 4.28 mmol), CuI (20.0 mg, 0.107 mmol), Pd(PPh₃)₄ (124 mg, 0.107 mmol), and THF (15 mL). After three cycles of vacuum/argon flash, the reaction mixture was heated at 100° C. for 1 h under microwave irradiation. After cooling down, the reaction mixture was filtered. The filtrate was concentrated and the residue was purified by prep-TLC eluting with petroleum ether/ethyl acetate (1:2) to afford the title compound as a yellow solid (180 mg, 49% yield over two steps).

Step 4—Synthesis of N-(tetrahydro-2H-pyran-4-yl)-3-(4-(trifluoromethyl)pyridin-2-yl)-1H-pyrazolo[4,3-c]pyridin-4-amine: A microwave vial equipped with a magnetic stirrer was charged with 1-(4-methoxybenzyl)-N-(tetrahydro-2H-pyran-4-yl)-3-(6-(trifluoromethyl)pyrimidin-4-yl)-1H-pyrazolo[4,3-c]pyridin-4-amine (100 mg, 0.210 mmol) and TFA (10 mL). The reaction mixture was heated at 120° C. for 2 h under microwave irradiation. After cooling down, the mixture was concentrated under reduced pressure. The residue was purified by reverse-phase prep-HPLC to afford the title compound as a pale green solid (35 mg, 47%). ¹H NMR (500 MHz, CD₃OD) δ 8.89 (d, J=5.0 Hz, 1H), 8.67 (s, 1H), 7.74-7.72 (m, 2H), 6.73 (d, J=5.0 Hz, 1H), 4.21-4.17 (m, 1H), 4.07-4.03 (m, 2H), 3.69-3.65 (m, 2H), 2.20-2.18 (m, 2H), 1.73-1.70 (m, 2H).

### 3-(pyrimidin-4-yl)-N-(tetrahydro-2H-pyran-4-yl)-1H-pyrazolo[4,3-c]pyridin-4-amine

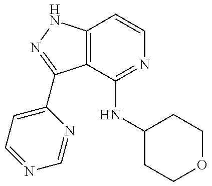 [SMILES: c1cc(-c2n[nH]c3ccnc(NC4CCOCC4)c23)ncn1]

Synthesis of 3-(pyrimidin-4-yl)-N-(tetrahydro-2H-pyran-4-yl)-1H-pyrazolo[4,3-c]pyridin-4-amine: The compound was prepared according to the general procedure described in the synthesis of N-ethyl-3-(pyrimidin-4-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 4,6-dichloropyrimidine to give the title compound as a yellow solid (19 mg, 16% yield over three steps). ¹H NMR (500 MHz, CD₃OD) δ 9.23 (d, J=1.0 Hz, 1H), 8.84 (d, J=1.0 Hz, 1H), 8.39 (dd, J=1.0, 5.0 Hz, 1H), 7.73 (d, J=6.5 Hz, 1H), 6.71 (d, J=6.5 Hz, 1H), 4.21-4.18 (m, 1H), 4.07-4.03 (m, 2H), 3.69-3.64 (m, 2H), 2.19-2.16 (m, 2H), 1.78-1.71 (m, 2H).

### 3-(pyridin-2-yl)-N-(tetrahydro-2H-pyran-4-yl)-1H-pyrazolo[4,3-c]pyridin-4-amine

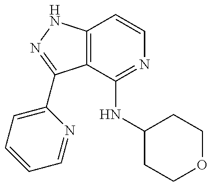 [SMILES: c1ccc(-c2n[nH]c3ccnc(NC4CCOCC4)c23)nc1]

Synthesis of 3-(pyridin-2-yl)-N-(tetrahydro-2H-pyran-4-yl)-1H-pyrazolo[4,3-c]pyridin-4-amine: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 2-bromopyridine to give the title compound as a white solid (86 mg, 37% yield over two steps). ¹H-NMR (500 MHz, CDCl₃) δ 10.62 (s, 1H), 10.00 (s, 1H), 8.58 (d, J=4.5 Hz, 1H), 8.38 (d, J=8.0 Hz, 1H), 7.89 (d, J=6.0 Hz, 1H), 7.86-7.83 (m, 1H), 7.35-7.32 (m, 1H), 6.58 (d, J=6.0 Hz, 1H), 4.41-4.35 (m, 1H), 4.07-4.03 (m, 2H), 3.69-3.66 (m, 2H), 2.21-2.20 (m, 2H), 1.75-1.71 (m, 2H).

### N-(tetrahydro-2H-pyran-4-yl)-3-(6-(trifluoromethyl)pyrimidin-4-yl)-1H-pyrazolo[4,3-c]pyridin-4-amine

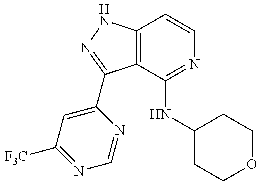 [SMILES: FC(F)(F)c1cc(-c2n[nH]c3ccnc(NC4CCOCC4)c23)ncn1]

Synthesis of N-(tetrahydro-2H-pyran-4-yl)-3-(6-(trifluoromethyl)pyrimidin-4-yl)-1H-pyrazolo[4,3-c]pyridin-4-amine: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 4-bromo-6-(trifluoromethyl)pyrimidine to give the title compound as a white solid (123 mg, 34% yield over two steps). ¹H NMR (500 MHz, CD₃OD) δ 9.45 (s, 1H), 8.72 (s, 1H), 7.73 (s, 1H), 6.75 (s, 1H), 4.22-4.19 (m, 1H), 4.08-4.05 (m, 2H), 3.69-3.64 (m, 2H), 2.19-2.17 (m, 2H), 1.80-1.73 (m, 2H).

### 2-(4-(tetrahydro-2H-pyran-4-ylamino)-1H-pyrazolo[4,3-c]pyridin-3-yl)isonicotinonitrile

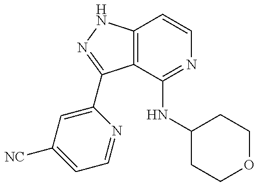 [SMILES: N#Cc1ccnc(-c2n[nH]c3ccnc(NC4CCOCC4)c23)c1]

Synthesis of 2-(4-(tetrahydro-2H-pyran-4-ylamino)-1H-pyrazolo[4,3-c]pyridin-3-yl) isonicotinonitrile: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 2-bromo-isonicotinonitrile to give the title compound as a white solid (80 mg, 26% yield over two steps). ¹H NMR (500 MHz, CD₃OD) δ 8.85 (d, J=5.0 Hz, 1H), 8.69 (s, 1H), 7.74-7.72 (m, 2H), 6.71 (d, J=5.0 Hz, 1H), 4.12-4.09 (m, 1H), 4.06-4.02 (m, 2H), 3.69-3.64 (m, 2H), 2.18-2.16 (m, 2H), 1.73-1.70 (m, 2H).

### 3-(6-methylpyridin-2-yl)-N-(tetrahydro-2H-pyran-4-yl)-1H-pyrazolo[4,3-c]pyridin-4-amine

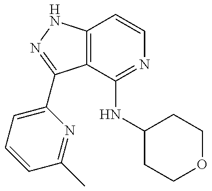 [SMILES: Cc1cccc(-c2n[nH]c3ccnc(NC4CCOCC4)c23)n1]

Synthesis of 3-(6-methylpyridin-2-yl)-N-(tetrahydro-2H-pyran-4-yl)-1H-pyrazolo[4,3-c]pyridin-4-amine: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 2-bromo-6-methylpyridine to give the title compound as a white solid (9.0 mg, 10% yield over two steps). ¹H NMR (500 MHz, CD₃OD) δ 8.19 (d, J=8.0 Hz, 1H), 7.83-7.80 (m, 1H), 7.71 (d, J=5.5 Hz, 1H), 7.29 (d, J=8.0 Hz, 1H), 6.69 (d, J=6.5 Hz, 1H), 4.33-4.28 (m, 1H), 4.08-4.05 (m, 2H), 3.65-3.60 (m, 2H), 2.73 (s, 3H), 2.15-2.12 (m, 2H), 1.81-1.73 (m, 2H).

### 3-(2-methylpyrimidin-4-yl)-N-(tetrahydro-2H-pyran-4-yl)-1H-pyrazolo[4,3-c]pyridin-4-amine

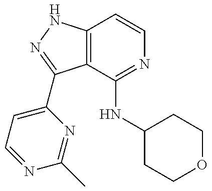 [SMILES: Cc1nccc(-c2n[nH]c3ccnc(NC4CCOCC4)c23)n1]

Synthesis of 3-(2-methylpyrimidin-4-yl)-N-(tetrahydro-2H-pyran-4-yl)-1H-pyrazolo[4,3-c]pyridin-4-amine: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 4-chloro-2-methylpyrimidine to give the title compound as a yellow solid (26 mg, 60% yield over two steps). ¹H NMR (500 MHz, CD₃OD) δ 8.72 (d, J=5.5 Hz, 1H), 8.21 (d, J=5.5 Hz, 1H), 7.75 (d, J=6.5 Hz, 1H), 6.72 (d, J=6.5 Hz, 1H), 4.27 (m, 1H), 4.05-4.08 (m, 2H), 3.60-3.65 (m, 2H), 2.861 (s, 3H), 2.15-2.18 (m, 2H), 1.76-1.73 (m, 2H).

### 3-(4-chloropyridin-2-yl)-N-(tetrahydro-2H-pyran-4-yl)-1H-pyrazolo[4,3-c]pyridin-4-amine

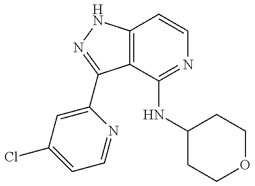 [SMILES: Clc1ccnc(-c2n[nH]c3ccnc(NC4CCOCC4)c23)c1]

Synthesis of 3-(4-chloropyridin-2-yl)-N-(tetrahydro-2H-pyran-4-yl)-1H-pyrazolo[4,3-c]pyridin-4-amine: The compound was prepared according to the general procedure described in the synthesis of N-(tetrahydro-2H-pyran-4-yl)-3-(4-(trifluoromethyl)pyridin-2-yl)-1H-pyrazolo[4,3-c]pyridin-4-amine by reacting 1-(4-methoxybenzyl)-N-(tetrahydro-2H-pyran-4-yl)-3-(trimethylstannyl)-1H-pyrazolo[4,3-c]pyridin-4-amine with 2-bromo-4-chloropyridine to give the title compound as a yellow solid (36 mg, 45% yield over two steps). ¹H NMR (500 MHz, CD₃OD) δ 8.60 (d, J=5.5 Hz, 1H), 8.43 (d, J=1.5 Hz, 1H), 7.75 (d, J=6.5 Hz, 1H), 7.51-7.50 (m, 1H), 6.68 (d, J=6.5 Hz, 1H), 4.20-4.18 (m, 1H), 4.06-4.03 (m, 2H), 3.69-3.64 (m, 2H), 2.18-2.15 (m, 2H), 1.75-1.68 (m, 2H).

### N-isopropyl-3-(6-(trifluoromethyl)pyrimidin-4-yl)-1H-pyrazolo[4,3-c]pyridin-4-amine

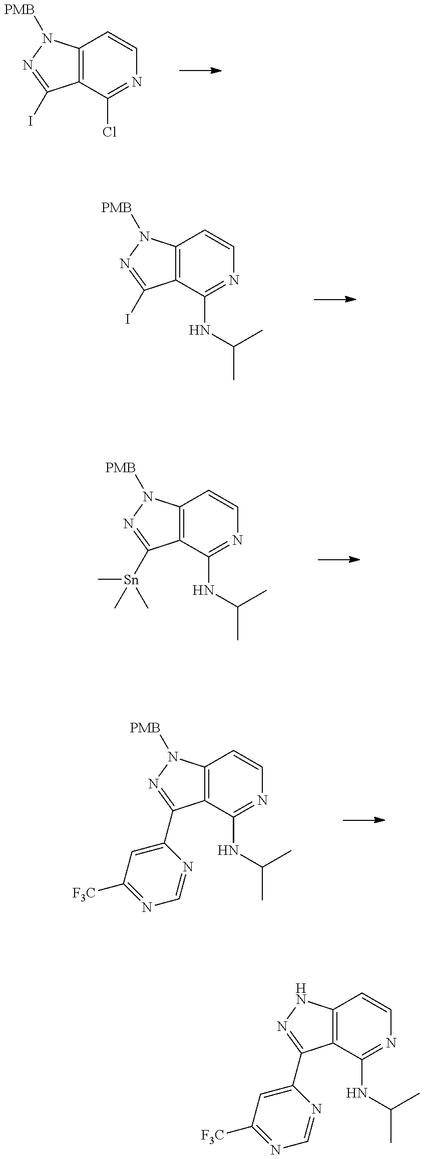

Step 1—Synthesis of 3-iodo-N-isopropyl-1-(4-methoxybenzyl)-1H-pyrazolo[4,3-c]pyridin-4-amine: A microwave vial equipped with a magnetic stirrer was charged with 4-chloro-3-iodo-1-(4-methoxybenzyl)-1H-pyrazolo[4,3-c]pyridine (2.00 g, 3.84 mmol), propan-2-amine (1.10 g, 19.2 mmol), n-BuOH (0.860 g, 7.68 mmol), and DIPEA (10 mL). The reaction mixture was heated at 170° C. for 2 h under microwave irradiation. After cooling down, the mixture was concentrated under reduced pressure. The residue was purified by silica gel column chromatography eluting with DCM/petroleum ether/TEA (2:1:0.01) to afford the title compound as a white solid (1.8 g, 85%).

Step 2—Synthesis of N-isopropyl-1-(4-methoxybenzyl)-3-(trimethylstannyl)-1H-pyrazolo[4,3-c]pyridin-4-amine: A reaction tube equipped with a magnetic stirrer was charged with 3-iodo-N-isopropyl-1-(4-methoxybenzyl)-1H-pyrazolo[4,3-c]pyridin-4-amine (750 mg, 1.78 mmol), 1,1,1,2,2,2-hexamethyldistannane (700 mg, 2.14 mmol), toluene (12 mL), and trans-Pd(PPh₃)₂Cl₂ (32 mg, 0.045 mmol). After three cycles of vacuum/argon flash, the reaction mixture was sealed and heated at 110° C. for 12 h. After cooling down, the mixture was filtered and the filtrate was concentrated under reduced pressure to give the crude title compound, which was used in the next step directly without further purification.

Step 3—Synthesis of N-isopropyl-1-(4-methoxybenzyl)-3-(6-(trifluoromethyl)pyrimidin-4-yl)-1H-pyrazolo[4,3-c]pyridin-4-amine: A microwave vial equipped with a magnetic stirrer was charged with N-isopropyl-1-(4-methoxybenzyl)-3-(trimethylstannyl)-1H-pyrazolo[4,3-c]pyridin-4-amine (310 mg, 0.670 mmol), 4-bromo-6-(trifluoromethyl)pyrimidine (120 mg, 0.530 mmol), LiCl (113 mg, 2.68 mmol), CuI (25.5 mg, 0.134 mmol), Pd(PPh₃)₄ (77 mg, 0.067 mmol), and THF (8 mL). After three cycles of vacuum/argon flash, the reaction mixture was heated at 100° C. for 30 min under microwave irradiation. The mixture was then cooled to room temperature and filtered. The filtrate was concentrated under reduced pressure and the resulting residue was purified by reverse-phase Combi-flash eluting with 0.3% NH₄HCO₃/CH₃CN (1:3) to afford the title compound as yellow oil (200 mg, 68%).

Step 4—Synthesis of N-isopropyl-3-(6-(trifluoromethyl)pyrimidin-4-yl)-1H-pyrazolo[4,3-c]pyridin-4-amine: A microwave vial equipped with a magnetic stirrer was charged with N-isopropyl-1-(4-methoxybenzyl)-3-(6-(trifluoromethyl)pyrimidin-4-yl)-1H-pyrazolo[4,3-c]pyridin-4-amine (200 mg, 0.450 mmol) and TFA (5 mL). The reaction mixture was heated at 150° C. for 2 h under microwave irradiation. After cooling down, the mixture was concentrated under reduced pressure. The residue was purified by reverse-phase prep-HPLC to afford the title compound as a yellow solid (67 mg, 46%). ¹H NMR (500 MHz, CD₃OD) δ 9.39 (s, 1H), 8.69 (d, J=1.5 Hz, 1H), 7.71 (d, J=6.0 Hz, 1H), 6.73 (d, J=6.0 Hz, 1H), 4.25 (t, J=6.5 Hz, 1H), 1.41 (d, J=6.5 Hz, 6H).

### 2-(4-(isopropylamino)-1H-pyrazolo[4,3-c]pyridin-3-yl)isonicotinonitrile

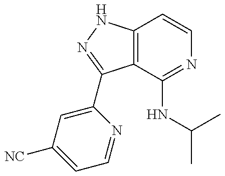 [SMILES: CC(C)Nc1nccc2[nH]nc(-c3cc(C#N)ccn3)c12]

Synthesis of 2-(4-(isopropylamino)-1H-pyrazolo[4,3-c]pyridin-3-yl)isonicotinonitrile: The compound was prepared according to the general procedure described in the synthesis of N-isopropyl-3-(6-(trifluoromethyl)pyrimidin-4-yl)-1H-pyrazolo[4,3-c]pyridin-4-amine by reacting N-isopropyl-1-(4-methoxybenzyl)-3-(trimethylstannyl)-1H-pyrazolo[4,3-c]pyridin-4-amine with 2-bromoisonicotinonitrile to give the title compound as a yellow solid (10 mg, 8.5% yield over two steps). ¹H NMR (500 MHz, CD₃OD) δ 8.80 (d, J=5.0 Hz, 1H), 8.63 (s, 1H), 7.69-7.66 (m, 2H), 6.64 (d, J=5.0 Hz, 1H), 3.32-3.29 (m, 1H), 1.36-1.34 (d, J=6.0 Hz, 6H).

### N-isopropyl-3-(4-(trifluoromethyl)pyridin-2-yl)-1H-pyrazolo[4,3-c]pyridin-4-amine

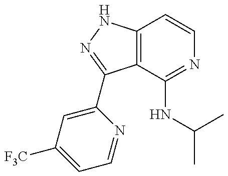 [SMILES: CC(C)Nc1nccc2[nH]nc(-c3cc(C(F)(F)F)ccn3)c12]

Synthesis of N-isopropyl-3-(4-(trifluoromethyl)pyridin-2-yl)-1H-pyrazolo[4,3-c]pyridin-4-amine: The compound was prepared according to the general procedure described in the synthesis of N-isopropyl-3-(6-(trifluoromethyl)pyrimidin-4-yl)-1H-pyrazolo[4,3-c]pyridin-4-amine by reacting N-isopropyl-1-(4-methoxybenzyl)-3-(trimethylstannyl)-1H-pyrazolo[4,3-c]pyridin-4-amine with 2-bromo-4-(trifluoromethyl)pyridine to give the title compound as a yellow solid (21 mg, 25% yield over two steps). ¹H NMR (500 MHz, CD₃OD) δ 8.87 (d, J=5.0 Hz, 1H), 8.64 (s, 1H), 7.71 (d, J=5.5 Hz, 2H), 6.68 (d, J=6.0 Hz, 1H), 4.24 (t, J=6.0 Hz, 1H), 1.40 (d, J=6.5 Hz, 6H).

### 3-(4-chloropyridin-2-yl)-N-isopropyl-1H-pyrazolo[4,3-c]pyridin-4-amine

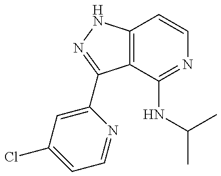 [SMILES: CC(C)Nc1nccc2[nH]nc(-c3cc(Cl)ccn3)c12]

Synthesis of 3-(4-chloropyridin-2-yl)-N-isopropyl-1H-pyrazolo[4,3-c]pyridin-4-amine: The compound was prepared according to the general procedure described in the synthesis of N-isopropyl-3-(6-(trifluoromethyl)pyrimidin-4-yl)-1H-pyrazolo[4,3-c]pyridin-4-amine by reacting N-isopropyl-1-(4-methoxybenzyl)-3-(trimethylstannyl)-1H-pyrazolo[4,3-c]pyridin-4-amine with 2-bromo-4-chloropyridine to give the title compound as a yellow solid (7.0 mg, 11% yield over two steps). ¹H NMR (500 MHz, CD₃OD) δ 8.54 (d, J=5.0 Hz, 1H), 8.39 (d, J=2.0 Hz, 1H), 7.66 (d, J=6.0 Hz, 1H), 7.46-7.45 (m, 1H), 6.63 (d, J=6.0 Hz, 1H), 4.19 (t, J=6.5 Hz, 1H), 1.40 (d, J=6.5 Hz, 6H).

### 3-(4-((3R,4R)-3,4-difluoropyrrolidin-1-yl)pyridin-2-yl)-N-isopropyl-1H-pyrazolo[4,3-c]pyridin-4-amine

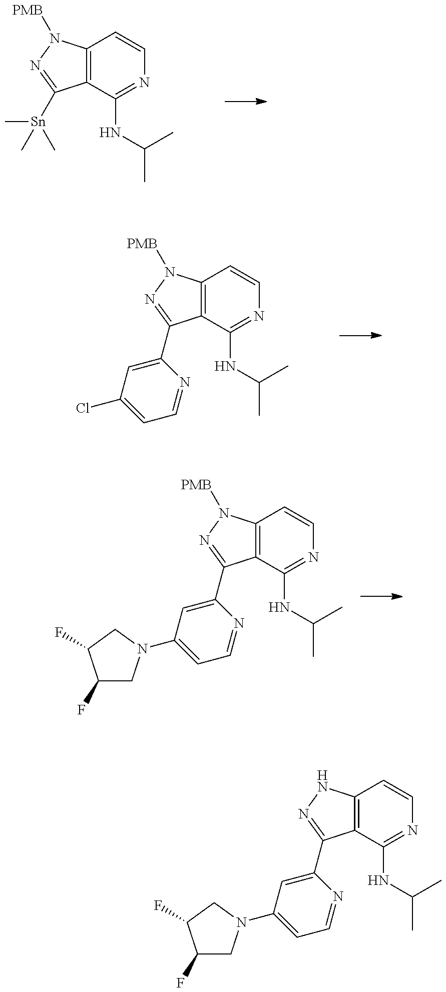

Step 1—Synthesis of 3-(4-chloropyridin-2-yl)-N-isopropyl-1-(4-methoxybenzyl)-1H-pyrazolo[4,3-c]pyridin-4-amine: A microwave vial equipped with a magnetic stirrer was charged with N-isopropyl-1-(4-methoxybenzyl)-3-(trimethylstannyl)-1H-pyrazolo[4,3-c]pyridin-4-amine (100 mg, 0.220 mmol), 2-bromo-4-chloropyridine (34 mg, 0.18 mmol), LiCl (37 mg, 0.88 mmol), CuI (8.4 mg, 0.044 mmol), Pd(PPh₃)₄ (26 mg, 0.022 mmol), and THF (4 mL). After three cycles of vacuum/argon flash, the reaction mixture was heated at 100° C. for 30 min under microwave irradiation. After cooling down, the mixture was filtered. The filtrate was concentrated under reduced pressure and the residue was purified by reverse-phase Combi-flash eluting with 0.3% NH₄HCO₃/CH₃CN (1:3) to afford the title compound as yellow oil (70 mg, 78%).

Step 2—Synthesis of 3-(4-((3R,4R)-3,4-difluoropyrrolidin-1-yl)pyridin-2-yl)-N-iso-propyl-1-(4-methoxybenzyl)-1H-pyrazolo[4,3-c]pyridin-4-amine: A mixture of 3-(4-chloropyridin-2-yl)-N-isopropyl-1-(4-methoxybenzyl)-1H-pyrazolo[4,3-c]pyridin-4-amine (200 mg, 0.490 mmol), (3R,4R)-3,4-difluoropyrrolidine hydrochloride (63 mg, 0.59 mmol), Pd₂(dba)₃ (45 mg, 0.049 mmol), Xantphos (57 mg, 0.098 mmol), cesium carbonate (559 mg, 1.72 mmol), dioxane (10 mL) was degassed with nitrogen. The mixture was heated to 140° C. for 1 h under microwave irradiation. The reaction mixture was filtered and concentrated. The residue was purified by silica gel column chromatograph eluting with petroleum ether/ethyl acetate (3:1) to afford the title compound as a yellow solid (95 mg, 41%).

Step 3—Synthesis of 3-(4-((3R,4R)-3,4-difluoropyrrolidin-1-yl)pyridin-2-yl)-N-isopropyl-1H-pyrazolo[4,3-c]pyridin-4-amine: A microwave vial equipped with a magnetic stirrer was charged with 3-(4-((3R,4R)-3,4-difluoropyrrolidin-1-yl)pyridin-2-yl)-N-isopropyl-1-(4-methoxybenzyl)-1H-pyrazolo[4,3-c]pyridin-4-amine (95.0 mg, 0.199 mmol) and TFA (3 mL). The reaction mixture was heated at 120° C. for 1 h under microwave irradiation. After cooling down, the mixture was concentrated under reduced pressure. The residue was purified by reverse-phase prep-HPLC to afford the title compound as a yellow solid (11 mg, 16%). ¹H NMR (500 MHz, CD₃OD) δ 8.27 (d, J=6.0 Hz, 1H), 7.64 (d, J=6.0 Hz, 1H), 7.55 (s, 1H), 6.68-6.64 (m, 2H), 5.49 (s, 1H), 5.38 (s, 1H), 4.19 (t, J=6.0 Hz, 1H), 3.87-3.79 (m, 4H), 1.38 (d, J=7.0 Hz, 6H).

### N-isopropyl-3-(4-(1-methyl-1H-pyrazol-4-yl)pyridin-2-yl)-1H-pyrazolo[4,3-c]pyridin-4-amine

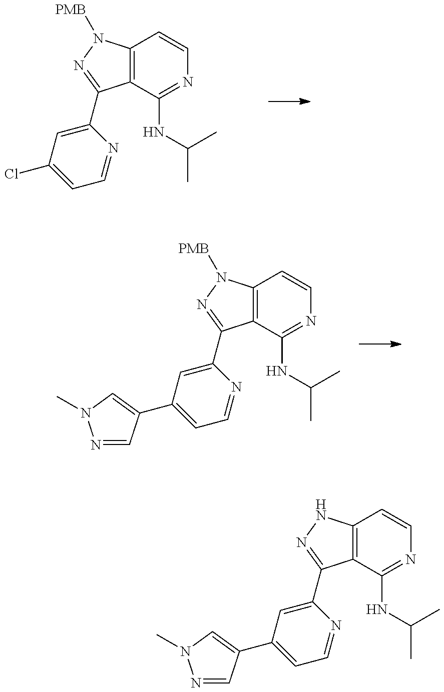

Step 1—Synthesis of N-isopropyl-1-(4-methoxybenzyl)-3-(4-(1-methyl-1H-pyrazol-4-yl)pyridin-2-yl)-1H-pyrazolo[4,3-c]pyridin-4-amine: A microwave vial equipped with a magnetic stirrer was charged with 3-(4-chloropyridin-2-yl)-N-isopropyl-1-(4-methoxybenzyl)-1H-pyrazolo[4,3-c]pyridin-4-amine (150 mg, 0.370 mmol), 1-methyl-4-(4,4,5,5-tetramethyl-1,3,2-dioxaborolan-2-yl)-1H-pyrazole (116 mg, 0.560 mmol), Pd(tBu₂PC₆H₄NMe₂)₂Cl₂ (26 mg, 0.037 mmol), Na₂CO₃ (156 mg, 1.48 mmol), CH₃CN (8 mL), and H₂O (0.8 mL). After three cycles of vacuum/argon flash, the reaction mixture was sealed and heated at 145° C. for 45 minutes under microwave irradiation. After cooling down, the mixture was concentrated under reduced pressure. The residue was purified by silica gel column chromatography eluting with DCM/MeOH (20:1) to give the title product as a yellow solid (80 mg, 48%).

Step 2—Synthesis of N-isopropyl-3-(4-(1-methyl-1H-pyrazol-4-yl)pyridin-2-yl)-1H-pyrazolo[4,3-c]pyridin-4-amine: A mixture of N-isopropyl-1-(4-methoxybenzyl)-3-(4-(1-methyl-1H-pyrazol-4-yl)pyridin-2-yl)-1H-pyrazolo[4,3-c]pyridin-4-amine (80 mg, 0.18 mmol) and TFA (15 mL) was stirred at reflux overnight. After cooling down and concentration, the mixture was adjusted pH around 7 by saturated NaHCO₃ solution. The resulting mixture was extracted by ethyl acetate (50 mL×2). The organic layer was washed by water (50 mL) and brine, dried over anhydrous Na₂SO₄, filtered, and evaporated in vacuo. The residue was purified by prep-HPLC to give the title product as a white solid (11 mg, 19%). ¹H NMR (500 MHz, CD₃OD) δ 8.57-8.55 (m, 2H), 8.29 (s, 1H), 8.09 (s, 1H), 7.66 (d, J=6.0 Hz, 1H), 7.62-7.61 (m, 1H), 6.68 (d, J=6.5 Hz, 1H), 4.23-4.20 (m, 1H), 4.00 (s, 3H), 1.41 (d, J=6.5 Hz, 6H).

### N-isopropyl-3-(4-(1-methyl-1H-pyrazol-3-yl)pyridin-2-yl)-1H-pyrazolo[4,3-c]pyridin-4-amine

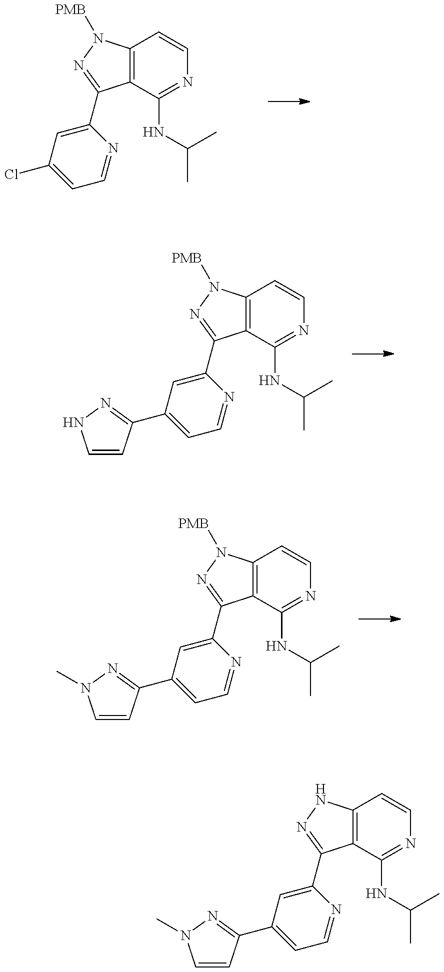

Step 1—Synthesis of 3-(4-(1H-pyrazol-3-yl)pyridin-2-yl)-N-isopropyl-1-(4-methoxybenzyl)-1H-pyrazolo[4,3-c]pyridin-4-amine: To a solution of 3-(4-chloropyridin-2-yl)-N-isopropyl-1-(4-methoxybenzyl)-1H-pyrazolo[4,3-c]pyridin-4-amine (100 mg, 0.246 mmol) and 3-(4,4,5,5-tetramethyl-1,3,2-dioxaborolan-2-yl)-1H-pyrazole (58.0 mg, 0.295 mmol) in CH₃CN (3 mL) was added Pd(tBu₂PC₆H₄NMe₂)₂Cl₂ (13 mg, 0.019 mmol) and aq. Na₂CO₃ solution (0.5 mL). After three cycles of vacuum/argon flash, the reaction mixture was sealed and heated at 120° C. for 2 h under microwave irradiation. The mixture was then diluted with ethyl acetate, washed with water and brine. The organic layer was dried and concentrated in vacuo. The residue was purified by silica gel column chromatography eluting with petroleum ether/ethyl acetate (5:1 to 1:1) to give the title product (65 mg, 60%).

Step 2—Synthesis of N-isopropyl-1-(4-methoxybenzyl)-3-(4-(1-methyl-1H-pyrazol-3-yl)pyridin-2-yl)-1H-pyrazolo[4,3-c]pyridin-4-amine: To a solution of 3-(4-(1H-pyrazol-3-yl)pyridin-2-yl)-N-isopropyl-1-(4-methoxybenzyl)-1H-pyrazolo[4,3-c]pyridin-4-amine (65.0 mg, 0.148 mmol) in DMF (1.0 mL) was added NaH (6.5 mg, 0.17 mmol) and iodomenthane (20.0 mg, 0.148 mmol) at 0° C. After being stirred for 5 h, the mixture was poured into water. The resulting mixture was extracted with ethyl acetate (30 mL×3). The organic layer was washed with water and brine, dried over Na₂SO₄, and concentrated in vacuo to give the title compound (72 mg, 100%).

Step 3—Synthesis of N-isopropyl-3-(4-(1-methyl-1H-pyrazol-3-yl)pyridin-2-yl)-1H-pyrazolo[4,3-c]pyridin-4-amine: A solution of N-isopropyl-1-(4-methoxybenzyl)-3-(4-(1-methyl-1H-pyrazol-3-yl)pyridin-2-yl)-1H-pyrazolo[4,3-c]pyridin-4-amine (72 mg, 0.16 mmol) in TFA (1 mL) was heated at 120° C. for 45 min under microwave irradiation. The reaction mixture was concentrated in vacuo. The residue was purified by prep-HPLC to give the title compound (24 mg, 46%). ¹H NMR (500 MHz, CDCl₃) δ 10.81 (s, 1H), δ 8.69 (s, 1H), 8.45 (d, J=4.5 Hz, 1H), 7.83 (d, J=5.5 Hz, 1H), 7.73 (d, J=5.0 Hz, 1H), 7.46 (s, 1H), 6.77 (s, 1H), 6.54 (d, J=6.5 Hz, 1H), 4.39-4.34 (m, 1H), 4.00 (s, 3H), 1.4 (t, J=2.5 Hz, 6H).

### N-isopropyl-3-(4-(1-methyl-1H-pyrazol-5-yl)pyridin-2-yl)-1H-pyrazolo[4,3-c]pyridin-4-amine

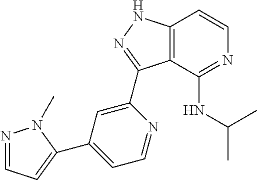 [SMILES: CC(C)Nc1nccc2[nH]nc(-c3cc(-c4ccnn4C)ccn3)c12]

Synthesis of N-isopropyl-3-(4-(1-methyl-1H-pyrazol-5-yl)pyridin-2-yl)-1H-pyrazolo[4,3-c]pyridin-4-amine: The compound was prepared according to the general procedure described in the synthesis of N-isopropyl-3-(4-(1-methyl-1H-pyrazol-4-yl)pyridin-2-yl)-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with 1-methyl-5-(4,4,5,5-tetramethyl-1,3,2-dioxaborolan-2-yl)-1H-pyrazole to give the title compound as a yellow solid (50 mg, 33% yield over two steps). ¹H NMR (500 MHz, CD₃OD) δ 8.69 (d, J=5.0 Hz, 1H), 8.46 (s, 1H), 7.65 (d, J=4.0 Hz, 1H), 7.56-7.54 (m, 2H), 6.64-6.61 (m, 2H), 4.48-4.21 (m, 1H), 4.01 (s, 3H), 1.36 (d, J=6.5 Hz, 6H).

### 3-(4-(1,5-dimethyl-4,5-dihydro-1H-pyrazol-4-yl)pyridin-2-yl)-N-isopropyl-1H-pyrazolo[4,3-c]pyridin-4-amine and 3-(4-(1,3-dimethyl-1H-pyrazol-4-yl)pyridin-2-yl)-N-isopropyl-1H-pyrazolo[4,3-c]pyridin-4-amine

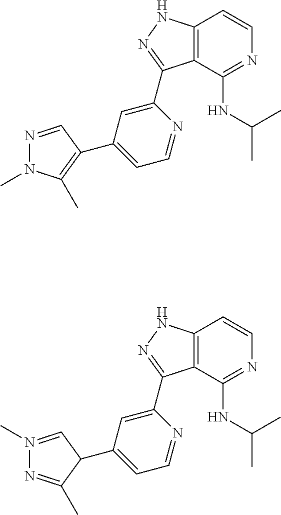

Synthesis of 3-(4-(1,5-dimethyl-4,5-dihydro-1H-pyrazol-4-yl)pyridin-2-yl)-N-isopropyl-1H-pyrazolo[4,3-c pyridin-4-amine and 3-(4-(1,3-dimethyl-1H-pyrazol-4-yl)pyridin-2-yl)-N-isopropyl-1H-pyrazolo[4,3-c]pyridin-4-amine: The compounds were prepared according to the general procedure described in the synthesis of N-isopropyl-3-(4-(1-methyl-1H-pyrazol-4-yl)pyridin-2-yl)-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with a mixture of 1,3-dimethyl-1H-pyrazol-4-ylboronic acid and 1,5-dimethyl-1H-pyrazol-4-ylboronic acid to give 3-(4-(1,5-dimethyl-4,5-dihydro-1H-pyrazol-4-yl)pyridin-2-yl)-N-isopropyl-1H-pyrazolo[4,3-c]pyridin-4-amine (11 mg, 2.3% yield over two steps) and 3-(4-(1,3-dimethyl-1H-pyrazol-4-yl)pyridin-2-yl)-N-isopropyl-1H-pyrazolo[4,3-c]pyridin-4-amine (GCP-AE20-032) (20 mg, 4.5% yield over two steps).

3-(4-(1,5-dimethyl-4,5-dihydro-1H-pyrazol-4-yl)pyridin-2-yl)-N-isopropyl-1H-pyrazolo[4,3-c]pyridin-4-amine: ¹H NMR (500 MHz, CDCl₃) δ 10.79 (s, 1H), 8.56 (d, J=5.5 Hz, 1H), 8.38 (s, 1H), 7.88 (d, J=5.5 Hz, 1H), 7.76 (s, 1H), 7.32-7.30 (m, 1H), 6.56 (d, J=6.0 Hz, 1H), 4.41-4.37 (m, 1H), 3.89 (s, 3H), 2.52 (s, 3H), 1.39 (t, J=6.5 Hz, 6H).

3-(4-(1,3-dimethyl-1H-pyrazol-4-yl)pyridin-2-yl)-N-isopropyl-1H-pyrazolo[4,3-c]pyridin-4-amine: ¹H NMR (500 MHz, CDCl₃) δ 8.48 (d, J=5.0 Hz, 1H), 8.40 (s, 1H), 7.87 (d, J=6.0 Hz, 1H), 7.71 (s, 1H), 7.34 (d, J=3.5 Hz, 1H), 6.59 (d, J=6.0 Hz, 1H), 4.44-4.38 (m, 1H), 3.92 (s, 3H), 2.52 (s, 3H), 1.40 (t, J=6.0 Hz, 6H).

### 3-(2-fluoro-3,4′-bipyridin-2′-yl)-N-isopropyl-1H-pyrazolo[4,3-c]pyridin-4-amine

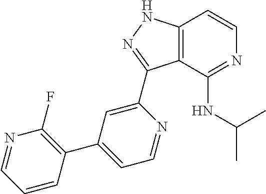 [SMILES: CC(C)Nc1nccc2[nH]nc(-c3cc(-c4cccnc4F)ccn3)c12]

Synthesis of 3-(2-fluoro-3,4′-bipyridin-2′-yl)-N-isopropyl-1H-pyrazolo[4,3-c]pyridin-4-amine: The compound was prepared according to the general procedure described in the synthesis of N-isopropyl-3-(4-(1-methyl-1H-pyrazol-4-yl)pyridin-2-yl)-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with 2-fluoro-3-(4,4,5,5-tetramethyl-1,3,2-dioxaborolan-2-yl)pyridine to give the title compound as a yellow solid (10 mg, 6.8% yield over two steps). ¹H-NMR (500 MHz, CD₃OD) δ 8.70 (s, 1H), 8.63 (d, J=4.5 Hz, 1H), 8.00 (d, J=5.5 Hz, 1H), 7.83 (d, J=5.5 Hz, 1H), 7.68 (d, J=6.5 Hz, 1H), 7.56 (d, J=5.5 Hz, 1H), 6.65-6.56 (m, 2H), 4.25-4.22 (m, 1H), 1.39 (d, J=6.5 Hz, 6H).

### 3-(4-cyclohexylpyridin-2-yl)-N-isopropyl-1H-pyrazolo[4,3-c]pyridin-4-amine

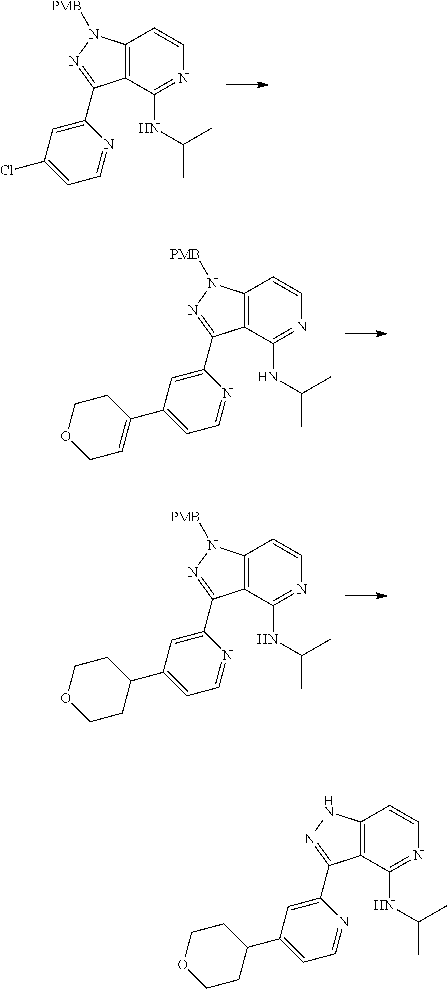

Step 1—Synthesis of 3-(4-cyclohexenylpyridin-2-yl)-N-isopropyl-1-(4-methoxybenzyl)-1H-pyrazolo[4,3-c]pyridin-4-amine: A microwave vial equipped with a magnetic stirrer was charged with 3-(4-chloropyridin-2-yl)-N-isopropyl-1-(4-methoxybenzyl)-1H-pyrazolo[4,3-c]pyridin-4-amine (350 mg, 0.860 mmol), 2-(cyclohex-2-enyl)-4,4,5,5-tetramethyl-1,3,2-dioxaborolane (266 mg, 1.29 mmol), Pd(tBu₂PC₆H₄NMe₂)₂Cl₂ (61 mg, 0.086 mmol), Na₂CO₃ (0.43 mL, 2M), and CH₃CN (10 mL). The suspension was heated at 145° C. for 45 min under microwave irradiation. After cooling down, the mixture was filtered. The filtrate was concentrated under reduced pressure and the resulting residue was purified by silica gel column chromatography eluting with petroleum ether/ethyl acetate (1:3) to afford the title compound as a yellow solid (80 mg, 20%).

Step 2—Synthesis of 3-(4-cyclohexylpyridin-2-yl)-N-isopropyl-1-(4-methoxybenzyl)-1H-pyrazolo[4,3-c]pyridin-4-amine: A three-neck flask equipped with a magnetic stirrer was charged with 3-(4-cyclohexenylpyridin-2-yl)-N-isopropyl-1-(4-methoxybenzyl)-1H-pyrazolo[4,3-c]pyridin-4-amine (60.0 mg, 0.132 mmol), Pd/C (30 mg), and MeOH (10 mL). The suspension was heated at 65° C. under H₂ for 12 h. After cooling down, the mixture was filtered. The filtrate was concentrated under reduced pressure to afford the title compound as a yellow solid (50 mg, 86%).

Step 3—Synthesis of 3-(4-cyclohexylpyridin-2-yl)-N-isopropyl-1H-pyrazolo[4,3-c]pyridin-4-amine: A microwave vial equipped with a magnetic stirrer was charged with 3-(4-cyclohexylpyridin-2-yl)-N-isopropyl-1-(4-methoxybenzyl)-1H-pyrazolo[4,3-c]pyridin-4-amine (44 mg, 0.096 mmol) and TFA (5 mL). The reaction mixture was heated at 110° C. for 30 min under microwave irradiation. After cooling down, the mixture was concentrated under reduced pressure. The residue was purified by reverse-phase prep-HPLC to afford the title compound as a yellow solid (6.4 mg, 20%). ¹H NMR (500 MHz, CD₃OD) δ 8.55 (d, J=5.0 Hz, 1H), 8.28 (s, 1H), 7.66 (d, J=6.0 Hz, 1H), 7.36-7.34 (m, 1H), 6.65 (d, J=6.5 Hz, 1H), 4.24-4.20 (m, 1H), 4.19-4.08 (m, 2H), 3.65-3.60 (m, 2H), 2.99-2.96 (m, 1H), 1.90-1.86 (m, 4H), 1.37 (d, J=6.0 Hz, 6H).

### N-isopropyl-3-(4-(tetrahydrofuran-3-yl)pyridin-2-yl)-1H-pyrazolo[4,3-c]pyridin-4-amine

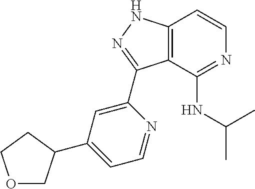 [SMILES: CC(C)Nc1nccc2[nH]nc(-c3cc(C4CCOC4)ccn3)c12]

Synthesis of N-isopropyl-3-(4-(tetrahydrofuran-3-yl)pyridin-2-yl)-1H-pyrazolo[4,3-c]pyridin-4-amine: The compound was prepared according to the general procedure described in the synthesis of 3-(4-cyclohexylpyridin-2-yl)-N-isopropyl-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with 2-(4,5-dihydrofuran-3-yl)-4,4,5,5-tetramethyl-1,3,2-dioxaborolane to give the title compound as a yellow solid (28 mg, 8.8% yield over three steps). ¹H NMR (500 MHz, CDCl₃) δ 10.55 (s, 1H), 8.49 (d, J=4.5 Hz, 1H), 8.26 (s, 1H), 7.88 (d, J=6.0 Hz, 1H), 7.22 (d, J=6.5 Hz, 1H), 6.57 (d, J=5.5 Hz, 1H), 4.39-4.43 (m, 1H), 4.40-4.38 (m, 2H), 3.93-3.98 (m, 1H), 3.84-3.87 (m, 1H), 3.47-3.52 (m, 1H), 2.43-2.50 (m, 1H), 2.06-2.12 (m, 1H), 1.39 (d, J=6.5 Hz, 6H).

### N-isopropyl-3-(4-(tetrahydrofuran-2-yl)pyridin-2-yl)-1H-pyrazolo[4,3-c]pyridin-4-amine

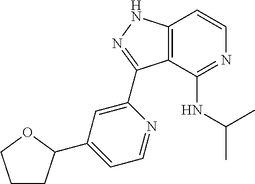 [SMILES: CC(C)Nc1nccc2[nH]nc(-c3cc(C4CCCO4)ccn3)c12]

Synthesis of N-isopropyl-3-(4-(tetrahydrofuran-2-yl)pyridin-2-yl)-1H-pyrazolo[4,3-c]pyridine-4-amine: The compound was prepared according to the general procedure described in the synthesis of 3-(4-cyclohexylpyridin-2-yl)-N-isopropyl-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with 2-(4,5-dihydrofuran-2-yl)-4,4,5,5-tetramethyl-1,3,2-dioxaborolane to give the title compound as a yellow solid (15 mg, 27% yield over three steps). ¹H NMR (500 MHz, CDCl₃) δ 8.48 (d, J=5.0 Hz, 1H), 8.32 (s, 1H), 7.84 (d, J=4.5 Hz, 1H), 7.35 (d, J=5.0 Hz, 1H), 6.65 (s, 1H), 5.00 (t, J=7.0 Hz, 1H), 4.49 (s, 1H), 4.16-4.12 (m, 1H), 4.03-4.00 (m, 1H), 2.48-2.44 (m, 1H), 2.06-2.02 (m, 2H), 1.86-1.81 (m, 2H), 1.43 (d, J=6.0 Hz, 6H).

### N-isopropyl-3-(4-(1-methylpiperidin-4-yl)pyridin-2-yl)-1H-pyrazolo[4,3-c]pyridin-4-amine

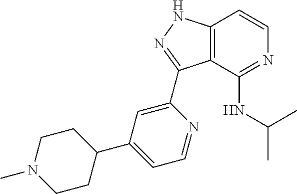 [SMILES: CC(C)Nc1nccc2[nH]nc(-c3cc(C4CCN(C)CC4)ccn3)c12]

Synthesis of N-isopropyl-3-(4-(1-methylpiperidin-4-yl)pyridin-2-yl)-1H-pyrazolo[4,3-c]pyridin-4-amine: The compound was prepared according to the general procedure described in the synthesis of 3-(4-cyclohexylpyridin-2-yl)-N-isopropyl-1H-pyrazolo[4,3-c]pyridin-4-amine by reacting 3-(4-chloropyridin-2-yl)-N-isopropyl-1-(4-methoxybenzyl)-1H-pyrazolo[4,3-c]pyridin-4-amine with 1-methyl-4-(4,4,5,5-tetramethyl-1,3,2-dioxaborolan-2-yl)-1,2,3,6-tetrahydropyridine to give the title compound as a yellow solid (11 mg, 6.7% yield over three steps). ¹H NMR (500 MHz, CD₃OD) δ 8.57 (d, J=5.0 Hz, 1H), 8.33 (s, 1H), 7.64 (d, J=6.0 Hz, 1H), 7.40-7.38 (m, 1H), 6.75 (d, J=6.5 Hz, 1H), 4.20-4.16 (m, 1H), 3.17 (d, J=12.0 Hz, 2H), 2.80 (t, J=6.5 Hz, 1H), 2.48 (s, 3H), 2.41 (t, J=11.5 Hz, 2H), 2.03 (d, J=13.5 Hz, 2H), 1.95-1.90 (m, 2H), 1.42 (d, J=13.5 Hz, 6H).

### 3-(6-cyclohexylpyrimidin-4-yl)-N-isopropyl-1H-pyrazolo[4,3-c]pyridin-4-amine

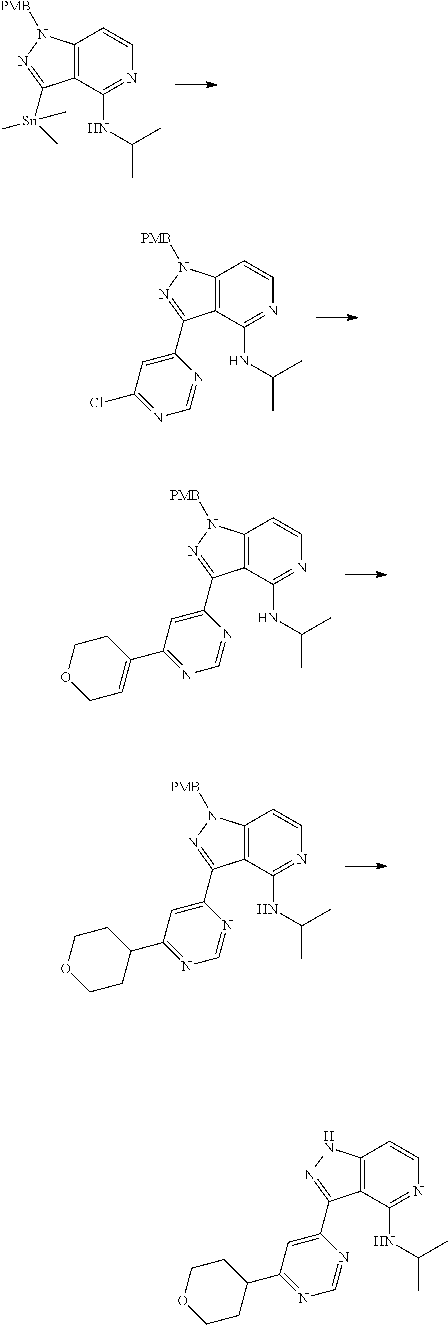

Step 1—Synthesis of 3-(6-chloropyrimidin-4-yl)-N-isopropyl-1-(4-methoxybenzyl)-1H-pyrazolo[4,3-c]pyridin-4-amine: A microwave vial equipped with a magnetic stirrer was charged with N-isopropyl-1-(4-methoxybenzyl)-3-(trimethylstannyl)-1H-pyrazolo[4,3-c]pyridin-4-amine (300 mg, 0.660 mmol), 4,6-dichloropyrimidine (195 mg, 1.32 mmol), LiCl (50 mg, 1.2 mmol), CuI (50 mg, 0.26 mmol), Pd(PPh₃)₄ (76 mg, 0.066 mmol), and THF (30 mL). After three cycles of vacuum/argon flash, the reaction mixture was heated at 100° C. for 30 min under microwave irradiation. After cooling down, the mixture was filtered. The filtrate was concentrated under reduced pressure and the residue was purified by silica gel column chromatography eluting with petroleum ether/ethyl acetate (1:2) to afford the title compound as a yellow solid (250 mg, 92%).

Step 2—Synthesis of 3-(6-cyclohexenylpyrimidin-4-yl)-N-isopropyl-1-(4-methoxybenzyl)-1H-pyrazolo[4,3-c]pyridin-4-amine: A microwave vial equipped with a magnetic stirrer was charged with 3-(6-chloropyrimidin-4-yl)-N-isopropyl-1-(4-methoxybenzyl)-1H-pyrazolo[4,3-c]pyridin-4-amine (200 mg, 0.490 mmol), 2-(cyclohex-2-enyl)-4,4,5,5-tetramethyl-1,3,2-dioxaborolane (144 mg, 0.740 mmol), Pd(tBu₂PC₆H₄NMe₂)₂Cl₂ (35 mg, 0.049 mmol), Na₂CO₃ (0.5 mL, 2M), and CH₃CN (6 mL). The suspension was heated at 170° C. for 30 min under microwave irradiation. After cooling down, the mixture was filtered. The filtrate was concentrated under reduced pressure and the residue was purified by silica gel column chromatography eluting with petroleum ether/ethyl acetate (1:1) to afford the title compound as a yellow solid (130 mg, 37%).

Step 3—Synthesis of 3-(6-cyclohexylpyrimidin-4-yl)-N-isopropyl-1-(4-methoxybenzyl)-1H-pyrazolo[4,3-c]pyridin-4-amine: A three-neck flask equipped with a magnetic stirrer was charged with 3-(6-cyclohexenylpyrimidin-4-yl)-N-isopropyl-1-(4-methoxybenzyl)-1H-pyrazolo[4,3-c]pyridin-4-amine (130 mg, 0.290 mmol), Pd/C (13 mg), and MeOH (10 mL). The suspension was heated at 65° C. under H₂ for 2 h. After cooling down, the mixture was filtered. The filtrate was concentrated under reduced pressure to afford the title compound as yellow oil (50 mg, 38%).

Step 4—Synthesis of 3-(6-cyclohexylpyrimidin-4-yl)-N-isopropyl-1H-pyrazolo[4,3-c]pyridin-4-amine: A microwave vial equipped with a magnetic stirrer was charged with 3-(6-cyclohexylpyrimidin-4-yl)-N-isopropyl-1-(4-methoxybenzyl)-1H-pyrazolo[4,3-c]pyridin-4-amine (50 mg, 0.11 mmol) and TFA (0.5 mL). The reaction mixture was heated at 150° C. for 30 min under microwave irradiation. After cooling down, the mixture was concentrated under reduced pressure. The residue was purified by reverse-phase prep-HPLC to afford the title compound as a yellow solid (13.8 mg, 72%). ¹H NMR (500 MHz, CD₃OD) δ 9.09 (d, J=1.5 Hz, 1H), 8.25 (d, J=1.0 Hz, 1H), 7.65 (d, J=7.0 Hz, 1H), 6.66 (d, J=7.5 Hz, 1H), 4.21-4.18 (m, 1H), 4.09-4.04 (m, 2H), 3.62-3.56 (m, 2H), 3.30-3.28 (m, 2H), 3.05-3.02 (m, 1H), 1.96-1.91 (m, 4H), 1.36 (d, J=8.5 Hz, 6H).

### N-isopropyl-3-(6-(tetrahydrofuran-3-yl)pyrimidin-4-yl)-1H-pyrazolo[4,3-c]pyridin-4-amine

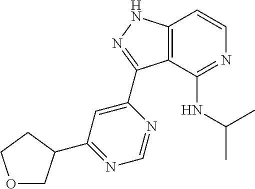 [SMILES: CC(C)Nc1nccc2[nH]nc(-c3cc(C4CCOC4)ncn3)c12]

Synthesis of N-isopropyl-3-(6-(tetrahydrofuran-3-yl)pyrimidin-4-yl)-1H-pyrazolo[4,3-c]pyridin-4-amine: The compound was prepared according to the general procedure described in the synthesis of 3-(6-cyclohexylpyrimidin-4-yl)-N-isopropyl-1H-pyrazolo[4,3-c]pyridin-4-amine by reacting 3-(6-chloropyrimidin-4-yl)-N-isopropyl-1-(4-methoxybenzyl)-1H-pyrazolo[4,3-c]pyridin-4-amine with 2-cyclopentenyl-4,4,5,5-tetramethyl-1,3,2-dioxaborolane to give the title compound as a yellow solid (4.0 mg, 2.6% yield over three steps). ¹H NMR (500 MHz, CDCl₃) δ 10.77 (s, 1H), 9.1 (s, 1H), 8.24 (s, 1H), 7.84 (d, J=6.0 Hz, 1H), 6.75-6.74 (d, J=5.0 Hz, 1H), 4.46 (d, J=7.0 Hz, 1H), 4.22-3.97 (m, 4H), 3.65-3.64 (m, 1H), 2.47-2.23 (m, 2H), 1.42 (d, J=6.0 Hz, 6H).

### N-isopropyl-3-(6-(tetrahydrofuran-2-yl)pyrimidin-4-yl)-1H-pyrazolo[4,3-c]pyridin-4-amine

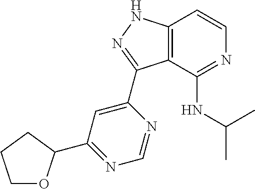 [SMILES: CC(C)Nc1nccc2[nH]nc(-c3cc(C4CCCO4)ncn3)c12]

Synthesis of N-isopropyl-3-(6-(tetrahydrofuran-2-yl)pyrimidin-4-yl)-1H-pyrazolo[4,3-c]pyridin-4-amine: The compound was prepared according to the general procedure described in the synthesis of 3-(6-cyclohexylpyrimidin-4-yl)-N-isopropyl-1H-pyrazolo[4,3-c]pyridin-4-amine by reacting 3-(6-chloropyrimidin-4-yl)-N-isopropyl-1-(4-methoxybenzyl)-1H-pyrazolo[4,3-c]pyridin-4-amine with 2-(4,5-dihydrofuran-2-yl)-4,4,5,5-tetramethyl-1,3,2-dioxaborolane to give the title compound as a yellow solid (4.1 mg, 2.6% yield over three steps). ¹H NMR (500 MHz, CD₃OD) δ 9.1 (d, J=1.5 Hz, 1H), 8.45 (s, 1H), 7.68 (s, 1H), 6.67 (d, J=6.0 Hz, 1H), 5.02-4.99 (m, 1H), 4.22-4.16 (m, 2H), 4.03-4.02 (m, 1H), 2.53-2.51 (m, 1H), 2.06-1.99 (m, 3H), 1.38 (d, J=6.0 Hz, 6H).

### 2-(4-(isopropylamino)-1H-pyrazolo[4,3-c]pyridin-3-yl)-N,N-dimethylisonicotinamide

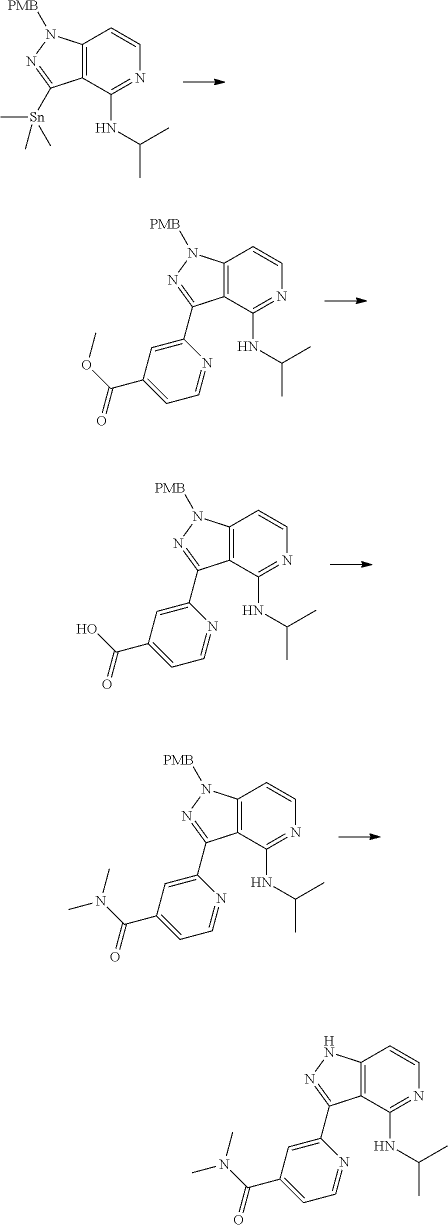

Step 1—Synthesis of methyl 2-(4-(isopropylamino)-1-(4-methoxybenzyl)-1H-pyrazolo[4,3-c]pyridin-3-yl)-isonicotinate: A microwave vial equipped with a magnetic stirrer was charged with N-isopropyl-1-(4-methoxybenzyl)-3-(trimethylstannyl)-1H-pyrazolo[4,3-c]pyridin-4-amine (2.4 g, 5.2 mmol), methyl 2-bromoisonicotinate (1.35 g, 6.25 mmol), LiCl (877 mg, 20.8 mmol), CuI (99.0 mg, 0.522 mole), Pd(PPh₃)₄ (201 mg, 0.174 mol), and THF (30 mL). After three cycles of vacuum/argon flash, the reaction mixture was heated at 100° C. for 0.5 hour under microwave irradiation. After cooling down, the mixture was filtered. The filtrate was concentrated under reduced pressure. The residue was purified by prep-TLC eluting with petroleum ether/ethyl acetate (1:2) to afford the title compound as a yellow solid (1.1 g, 38%).

Step 2—Synthesis of 2-(4-(isopropylamino)-1-(4-methoxybenzyl)-1H-pyrazolo[4,3-c]pyridin-3-yl)isonicotinic acid: To a mixture of methyl 2-(4-(isopropylamino)-1-(4-methoxybenzyl)-1H-pyrazolo[4,3-c]pyridin-3-yl)isonicotinate (704 mg, 1.63 mmol) and LiOH (321 mg, 7.16 mmol) in THF (20 mL) and methane (20 mL), was added H₂O (10 mL). The resulting mixture was stirred at room temperature for 4 h. After removal of the volatiles, the residue was treated with H₂O (30 mL) and ethyl acetate (40 mL). The precipitate was formed. The solid was collected by filtration to give the title compound as a white solid (640 mg, 94%).

Step 3—Synthesis of 2-(4-(isopropylamino)-1-(4-methoxybenzyl)-1H-pyrazolo[4,3-c]pyridin-3-yl)-N,N-dimethylisonicotinamide: A mixture of 2-(4-(isopropylamino)-1-(4-methoxybenzyl)-1H-pyrazolo[4,3-c]pyridin-3-yl)isonicotinic acid (200 mg, 0.480 mmol), dimethylamine hydrochloride (59 mg, 0.72 mmol), HATU (274 mg, 0.720 mmol), and Et₃N (145 mg, 1.44 mmol) in DMF (3 mL) was stirred at room temperature overnight. After removal of the volatiles, the residue was purified by prep-HPLC to afford the title compound as a yellow solid (130 mg, 55%).

Step 4—Synthesis of 2-(4-(isopropylamino)-1H-pyrazolo[4,3-c]pyridin-3-yl)-N,N-dimethylisonicotinamide: A microwave vial equipped with a magnetic stirrer was charged with 2-(4-(isopropylamino)-1-(4-methoxybenzyl)-1H-pyrazolo[4,3-c]pyridin-3-yl)-N,N-dimethylisonicotinamide (130 mg, 0.293 mmol) and TFA (3 mL). The reaction mixture was heated at 120° C. for 2 h under microwave irradiation. After cooling down, the mixture was concentrated under reduced pressure. The residue was purified by prep-HPLC to afford the title compound as a yellow solid (36 mg, 38%). ¹H NMR (500 MHz, DMSO) δ 13.45 (s, 1H), 10.17 (d, J=6.0 Hz, 1H), 8.73 (d, J=4.0 Hz, 1H), 8.23 (s, 1H), 7.76 (d, J=4.0 Hz, 1H), 7.47-7.45 (m, 1H), 6.60 (d, J=5.5 Hz, 1H), 4.26-4.22 (m, 1H), 3.03 (s, 3H), 2.93 (s, 3H), 1.31 (d, J=6.5 Hz, 6H).

### 2-(4-(isopropylamino)-1H-pyrazolo[4,3-c]pyridin-3-yl)pyridin-4-yl)(morpholino)methanone

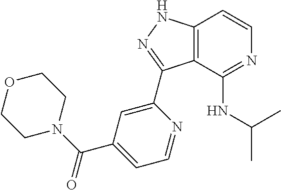 [SMILES: CC(C)Nc1nccc2[nH]nc(-c3cc(C(=O)N4CCOCC4)ccn3)c12]

Synthesis of (2-(4-(isopropylamino)-1H-pyrazolo[4,3-c]pyridin-3-yl)pyridin-4-yl)(morpholino)methanone: The compound was prepared according to the general procedure described in the synthesis of 2-(4-(isopropylamino)-1H-pyrazolo[4,3-c]pyridin-3-yl)-N,N-dimethylisonicotinamide by reacting 2-(4-(isopropylamino)-1-(4-methoxybenzyl)-1H-pyrazolo[4,3-c]pyridin-3-yl)isonicotinic acid with morpholine to give the title compound as a yellow solid (15.7 mg, 16% yield over two steps). ¹H NMR (500 MHz, CD₃OD) δ 8.74 (d, J=5.0 Hz, 1H), 8.38 (s, 1H), 7.69 (d, J=6.0 Hz, 1H), 7.45 (d, J=5.0 Hz, 5H), 6.67 (d, J=6.0 Hz, 1H), 4.23 (t, J=6.0 Hz, 1H), 3.82 (s, 4H), 3.68 (s, 2H), 3.48 (s, 2H), 1.40 (d, J=6.5 Hz, 6H).

### 6-(4-(isopropylamino)-1H-pyrazolo[4,3-c]pyridin-3-yl)-N,N-dimethylpyrimidine-4-carboxamide

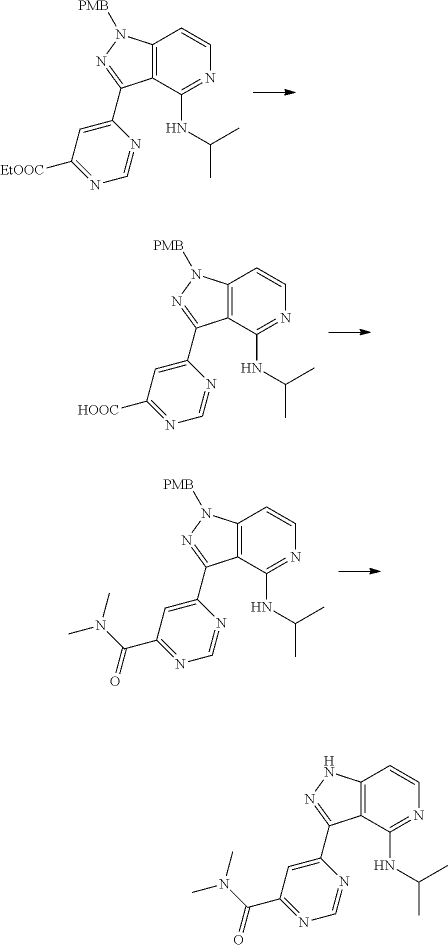

Step 1—Synthesis of 6-(4-(isopropylamino)-1-(4-methoxybenzyl)-1H-pyrazolo[4,3-c]pyridin-3-yl)pyrimidine-4-carboxylic acid: To a solution of ethyl 6-(4-(isopropylamino)-1-(4-methoxybenzyl)-1H-pyrazolo[4,3-c]pyridin-3-yl)pyrimidine-4-carboxylate (140 mg, 0.312 mmol) in MeOH (2 mL) was added lithium hydroxide solution (2 mL, 2 M in water). The mixture was stirred at room temperature for 2 h. After removal of the volatiles, the residue was purified by reverse phase Combi-flash to give the title compound as white solid (100 mg, 76%).

Step 2—Synthesis of 6-(4-(isopropylamino)-1-(4-methoxybenzyl)-1H-pyrazolo[4,3-c]pyridin-3-yl)-N,N-dimethylpyrimidine-4-carboxamide: To a solution of 6-(4-(isopropylamino)-1-(4-methoxybenzyl)-1H-pyrazolo[4,3-c]pyridin-3-yl)pyrimidine-4-carboxylic acid (100 mg, 0.240 mmol) in methylene chloride (20 mL) was added oxalyl chloride (10 mL) and DMF (0.5 mL). The mixture was stirred at room temperature for 2 hours. After removal of the volatiles, the acid chloride was treated with dimethylamine hydrochloride (40 mg, 0.48 mmol). After being stirred for 30 minutes at room temperature, the resulting mixture was concentrated. The residue was washed with water, extracted with DCM. The organic layer was dried and concentrated in vacuo to afford the title compound as a white solid (100 mg, 90%).

Step 3—Synthesis of 6-(4-(isopropylamino)-1H-pyrazolo[4,3-c]pyridin-3-yl)-N,N-dimethylpyrimidine-4-carboxamide: A microwave vial equipped with a magnetic stirrer was charged with 6-(4-(isopropylamino)-1-(4-methoxybenzyl)-1H-pyrazolo[4,3-c]pyridin-3-yl)-N,N-dimethylpyrimidine-4-carboxamide (50 mg, 0.11 mmol) and TFA (2 mL). The reaction mixture was heated at 120° C. for 30 min under microwave irradiation. After cooling down, the mixture was concentrated under reduced pressure. The residue was purified by reverse-phase prep-HPLC to afford the title compound as a yellow solid (12 mg, 34%). ¹H-NMR (500 MHz, CD₃OD) δ 9.25 (s, 1H), 8.47 (s, 1H), 7.68 (d, J=5.5 Hz, 1H), 6.68 (d, J=5.5 Hz, 1H), 4.24-4.21 (m, 1H), 3.15 (s, 3H), 3.08 (s, 3H), 1.38 (d, J=6.0 Hz, 6H).

### N,N-dimethyl-2-(4-(tetrahydro-2H-pyran-4-ylamino)-1H-pyrazolo[4,3-c]pyridin-3-yl)isonicotinamide

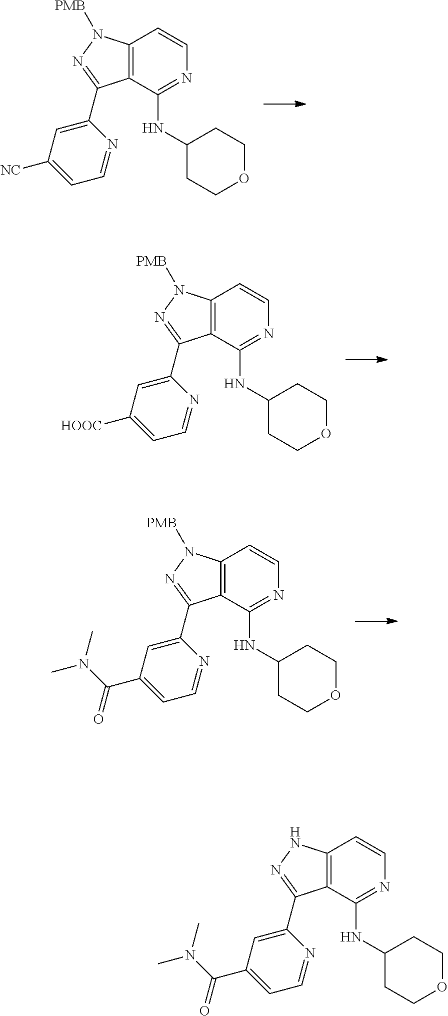

Step 1—Synthesis of 2-(1-(4-methoxybenzyl)-4-(tetrahydro-2H-pyran-4-ylamino)-1H-pyrazolo[4,3-c]pyridin-3-yl)isonicotinic acid: A microwave vial equipped with a magnetic stirrer was charged with 2-(1-(4-methoxybenzyl)-4-(tetrahydro-2H-pyran-4-ylamino)-1H-pyrazolo[4,3-c]pyridin-3-yl)isonicotinonitrile (250 mg, 0.570 mmol), KOH (3.3 g, 50 mmol), and H₂O (16.5 mL). The reaction mixture was heated at 100° C. for 2 h under microwave irradiation. After cooling down, the mixture was concentrated under reduced pressure. The residue was purified by reverse-phase prep-HPLC to afford the title compound as a white solid (230 mg, 88%).

Step 2—Synthesis of 2-(1-(4-methoxybenzyl)-4-(tetrahydro-2H-pyran-4-ylamino)-1H-pyrazolo[4,3-c]pyridin-3-yl)-N,N-dimethylisonicotinamide: To a 25-mL round bottom flash was charge with 2-(1-(4-methoxybenzyl)-4-(tetrahydro-2H-pyran-4-ylamino)-1H-pyrazolo[4,3-c]pyridin-3-yl)isonicotinic acid (200 mg, 0.440 mmol), dimethylamine (72 mg, 0.88 mmol), HATU (333 mg, 0.880 mmol), DIPEA (0.5 mL), and DMF (20 mL). The reaction mixture was stirred at room temperature for 12 h. After removal of the volatiles, the residue was purified by reverse-phase Combi-flash eluting with 0.3% NH₄HCO₃/CH₃CN (1:3) to afford the title compound as a white solid (186 mg, 87%).

Step 3—Synthesis of N,N-dimethyl-2-(4-(tetrahydro-2H-pyran-4-ylamino)-1H-pyrazolo[4,3-c]pyridin-3-yl)isonicotinamide: A microwave vial equipped with a magnetic stirrer was charged with 2-(1-(4-methoxybenzyl)-4-(tetrahydro-2H-pyran-4-ylamino)-1H-pyrazolo[4,3-c]pyridin-3-yl)-N,N-dimethylisonicotinamide (186 mg, 0.390 mmol) and TFA (20 mL). The reaction mixture was heated at 150° C. for 2 h under microwave irradiation. After removal of the volatiles, the residue was purified by reverse-phase prep-HPLC to afford the title compound as a white solid (86 mg, 60%). ¹H NMR (500 MHz, CD₃OD) δ 8.55 (d, J=4.5 Hz, 1H), 8.40 (s, 1H), 7.70 (d, J=6.0 Hz, 1H), 7.46 (d, J=5.0 Hz, 1H), 6.72 (d, J=6.5 Hz, 1H), 4.21-4.17 (m, 1H), 4.07-4.05 (m, 2H), 3.69-3.65 (m, 2H), 3.18 (s, 3H), 3.06 (s, 3H), 2.22-2.17 (m, 2H), 1.78-1.70 (m, 2H).

### Azetidin-1-yl(4-(tetrahydro-2H-pyran-4-ylamino)-1H-pyrazolo[4,3-c]pyridin-3-yl)pyridin-4-yl)methanone

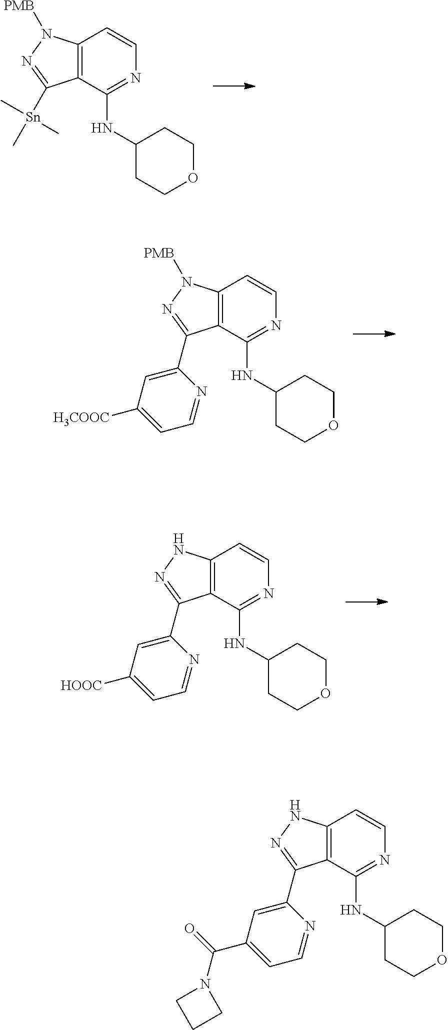

Step 1—Synthesis of methyl 2-(1-(4-methoxybenzyl)-4-(tetrahydro-2H-pyran-4-ylamino)-1H-pyrazolo[4,3-c]pyridin-3-yl)isonicotinate: A microwave vial equipped with a magnetic stirrer was charged with 1-(4-methoxybenzyl)-N-(tetrahydro-2H-pyran-4-yl)-3-(trimethylstannyl)-1H-pyrazolo[4,3-c]pyridin-4-amine (200 mg, 0.399 mmol), methyl 2-bromoisonicotinate (172 mg, 0.798 mmol), LiCl (67 mg, 1.6 mmol), CuI (7.6 mg, 0.040 mmol), Pd(PPh₃)₄ (46 mg, 0.040 mmol), and THF (10 mL). After three cycles of vacuum/argon flash, the reaction mixture was heated at 100° C. for 1 h under microwave irradiation. After cooling down, the mixture was concentrated under reduced pressure. The residue was purified by prep-TLC eluting with petroleum ether/ethyl acetate (2:1) to afford the title compound as a yellow solid (86 mg, 46%).

Step 2—Synthesis of 2-(4-(tetrahydro-2H-pyran-4-ylamino)-1H-pyrazolo[4,3-c]pyridin-3-yl)isonicotinic acid: A microwave vial equipped with a magnetic stirrer was charged with methyl 2-(1-(4-methoxybenzyl)-4-(tetrahydro-2H-pyran-4-ylamino)-1H-pyrazolo[4,3-c]pyridin-3-yl)isonicotinate (86.0 mg, 0.181 mmol) and TFA (12 mL). The reaction mixture was heated at 150° C. for 2 h under microwave irradiation. After cooling down, the mixture was concentrated under reduced pressure. The residue was purified by reverse-phase prep-HPLC to afford the title compound as a green solid (50 mg, 81%).

Step 3—Synthesis of azetidin-1-yl(2-(4-(tetrahydro-2H-pyran-4-ylamino)-1H-pyrazolo[4,3-c]pyridin-3-yl)pyridin-4-yl)methanone: To a 25-mL round bottom flash was charge with 2-(4-(tetrahydro-2H-pyran-4-ylamino)-1H-pyrazolo[4,3-c]pyridin-3-yl)isonicotinic acid (50 mg, 0.15 mmol), azetidine hydrochloride (28 mg, 0.30 mmol), HATU (96.5 mg, 0.254 mmol), DIPEA (0.5 mL), and DMF (3 mL). The reaction mixture was stirred at room temperature for 12 h. After removal of the volatiles, the residue was purified by reverse-phase Combi-flash eluting with 0.3% NH₄HCO₃/CH₃CN (1:3) to afford the title compound as a white solid (30 mg, 54%). ¹H NMR (500 MHz, CD₃OD) δ 8.76 (d, J=5.0 Hz, 1H), 8.57 (s, 1H), 7.72 (d, J=6.5 Hz, 1H), 7.61-7.60 (m, 1H), 6.69 (d, J=6.5 Hz, 1H), 4.49-4.42 (m, 2H), 4.42-4.17 (m, 2H), 4.07-4.03 (m, 1H), 3.69-3.64 (m, 2H), 3.34-3.32 (m, 2H), 2.48-2.42 (m, 2H), 2.19-2.16 (m, 2H), 1.76-1.70 (m, 2H).

### 3-(4-ethoxypyridin-2-yl)-N-(tetrahydro-2H-pyran-4-yl)-1H-pyrazolo[4,3-c]pyridin-4-amine

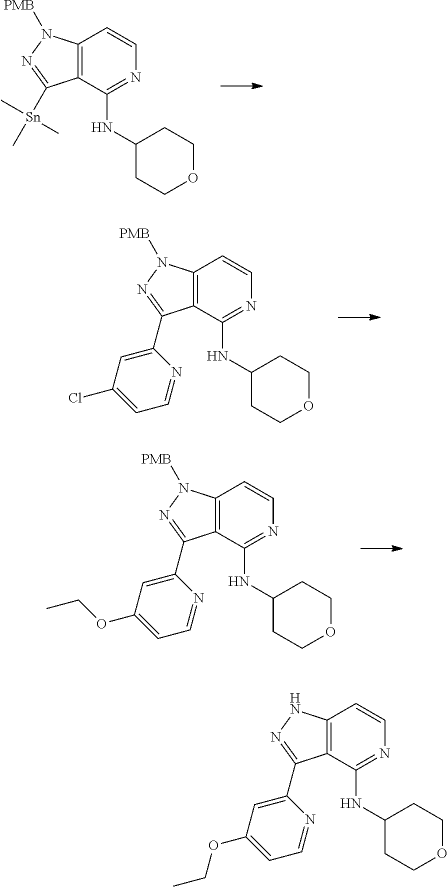

Step 1—Synthesis of 3-(4-chloropyridin-2-yl)-1-(4-methoxybenzyl)-N-(tetrahydro-2H-pyran-4-yl)-1H-pyrazolo[4,3-c]pyridin-4-amine: A microwave vial equipped with a magnetic stirrer was charged with 1-(4-methoxybenzyl)-N-(tetrahydro-2H-pyran-4-yl)-3-(trimethylstannyl)-1H-pyrazolo[4,3-c]pyridin-4-amine (500 mg, 0.990 mmol), 2-bromo-4-chloropyridine (287 mg, 1.49 mmol), LiCl (166 mg, 3.96 mmol), CuI (19 mg, 0.099 mmol), Pd(PPh₃)₄ (114 mg, 0.0990 mmol), and THF (20 mL). After three cycles of vacuum/argon flash, the reaction mixture was heated at 100° C. for 1 h under microwave irradiation. After cooling down, the mixture was concentrated under reduced pressure. The residue was purified by prep-TLC eluting with petroleum ether/ethyl acetate (3:1) to afford the title compound as a white solid (230 mg, 52%).

Step 2—Synthesis of 3-(4-ethoxypyridin-2-yl)-1-(4-methoxybenzyl)-N-(tetrahydro-2H-pyran-4-yl)-1H-pyrazolo[4,3-c]pyridin-4-amine: A microwave vial equipped with a magnetic stirrer was charged with 3-(4-chloropyridin-2-yl)-1-(4-methoxybenzyl)-N-(tetrahydro-2H-pyran-4-yl)-1H-pyrazolo[4,3-c]pyridin-4-amine (200 mg, 0.445 mmol), sodium ethanolate (61 mg, 0.89 mmol), and THF (20 ml). The reaction mixture was heated at 100° C. for 1 h under microwave irradiation. After cooling down, the mixture was concentrated under reduced pressure. The residue was purified by reverse-phase prep-HPLC to afford the title compound as a white solid (160 mg, 78%).

Step 3—Synthesis of 3-(4-ethoxypyridin-2-yl)-N-(tetrahydro-2H-pyran-4-yl)-1H-pyrazolo[4,3-c]pyridin-4-amine: A microwave vial equipped with a magnetic stirrer was charged with (3-(4-ethoxypyridin-2-yl)-1-(4-methoxybenzyl)-N-(tetrahydro-2H-pyran-4-yl)-1H-pyrazolo[4,3-c]pyridin-4-amine (160 mg, 0.350 mmol) and TFA (20 mL). The reaction mixture was heated at 150° C. for 2 h under microwave irradiation. After cooling down, the mixture was concentrated under reduced pressure. The residue was purified by reverse-phase prep-HPLC to afford the title compound as a white solid (86 mg, 72%). ¹H-NMR (500 MHz, CD₃OD) δ 8.45 (d, J=6.0 Hz, 1H), 7.90 (d, J=2.0 Hz, 1H), 7.69 (d, J=6.5 Hz, 1H), 7.00-6.98 (m, 1H), 6.67 (d, J=6.0 Hz, 1H), 4.28-4.24 (m, 2H), 4.20-4.16 (m, 1H) 4.07-4.03 (m, 2H), 3.69-3.64 (m, 2H), 2.17-2.15 (m, 2H), 1.76-1.69 (m, 2H), 1.50-1.47 (m, 3H).

### 3-(4-(2,2,6,6-tetrafluoromorpholino)pyridin-2-yl)-N-(tetrahydro-2H-pyran-4-yl)-1H-pyrazolo[4,3-c]pyridin-4-amine

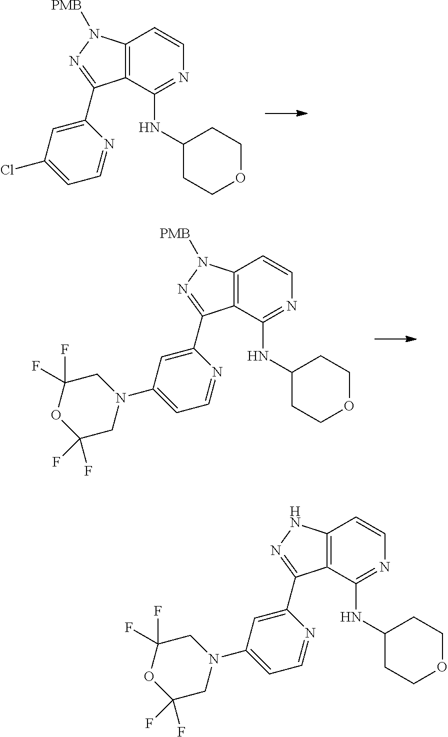

Step 1—Synthesis of 1-(4-methoxybenzyl)-3-(4-(2,2,6,6-tetrafluoromorpholino)pyridin-2-yl)-N-(tetrahydro-2H-pyran-4-yl)-1H-pyrazolo[4,3-c]pyridin-4-amine: A microwave vial equipped with a magnetic stirrer was charged with 3-(4-chloropyridin-2-yl)-1-(4-methoxybenzyl)-N-(tetrahydro-2H-pyran-4-yl)-1H-pyrazolo[4,3-c]pyridin-4-amine (150 mg, 0.330 mmol), Pd₂(dba)₃ (30 mg, 0.033 mmol), Xantphos (382 mg, 0.660 mmol), Cs₂CO₃ (216 mg, 0.660 mmol), 2,2,6,6-tetrafluoromorpholine (105 mg, 0.660 mmol), and dioxane (20 mL). The reaction mixture was heated at 140° C. for 1 h under microwave irradiation. After cooling down, the mixture was concentrated under reduced pressure. The residue was purified by reverse-phase prep-HPLC to afford the title compound as a white solid (86 mg, 46%).

Step 2—Synthesis of 3-(4-(2,2,6,6-tetrafluoromorpholino)pyridin-2-yl)-N-(tetrahydro-2H-pyran-4-yl)-1H-pyrazolo[4,3-c]pyridin-4-amine: A microwave vial equipped with a magnetic stirrer was charged with 1-(4-methoxybenzyl)-3-(4-(2,2,6,6-tetrafluoromorpholino)pyridin-2-yl)-N-(tetrahydro-2H-pyran-4-yl)-1H-pyrazolo[4,3-c]pyridin-4-amine (86 mg, 0.15 mmol) and TFA (20 mL). The reaction mixture was heated at 120° C. for 2 h under microwave irradiation. After cooling down, the mixture was concentrated under reduced pressure. The residue was purified by reverse-phase prep-HPLC to afford the title compound as a white solid (50 mg, 74%). ¹H NMR (500 MHz, CD₃OD) δ 8.41 (d, J=6.0 Hz, 1H), 7.94 (d, J=2.5 Hz, 1H), 7.69 (d, J=6.0 Hz, 1H), 7.08-7.06 (m, 1H), 6.66 (d, J=6.5 Hz, 1H), 4.24 (t, J=8.5 Hz, 4H), 4.21-4.16 (m, 1H), 4.06-4.03 (m, 2H), 3.69-3.64 (m, 2H), 2.16 (d, J=8.0 Hz, 2H), 1.75-1.67 (m, 2H).

### N,N,2-trimethyl-6-(4-(tetrahydro-2H-pyran-4-ylamino)-1H-pyrazolo[4,3-c]pyridin-3-yl)isonicotinamide

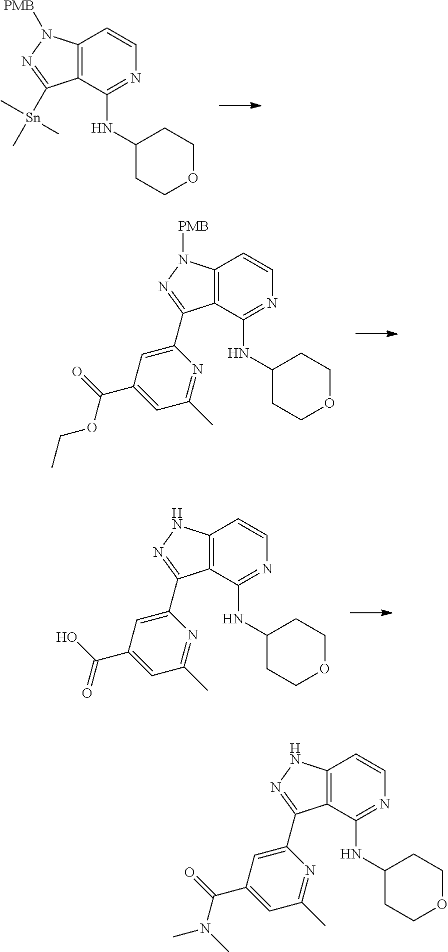

Step 1—Synthesis of ethyl 2-(1-(4-methoxybenzyl)-4-(tetrahydro-2H-pyran-4-ylamino)-1H-pyrazolo[4,3-c]pyridin-3-yl)-6-methylisonicotinate: A microwave vial equipped with a magnetic stirrer was charged with 1-(4-methoxybenzyl)-N-(tetrahydro-2H-pyran-4-yl)-3-(trimethylstannyl)-1H-pyrazolo[4,3-c]pyridin-4-amine (500 mg, 0.990 mmol), ethyl 2-bromo-6-methylisonicotinate (362 mg, 1.49 mmol), LiCl (166 mg, 3.96 mmol), CuI (19 mg, 0.099 mmol), Pd(PPh₃)₄ (114 mg, 0.0990 mmol), and THF (20 mL). After three cycles of vacuum/argon flash, the reaction mixture was heated at 100° C. for 1 h under microwave irradiation. After cooling down, the mixture was concentrated under reduced pressure. The residue was purified by prep-TLC eluting with petroleum ether/ethyl estate (2:1) to afford the title compound as a white solid (200 mg, 40%).

Step 2—Synthesis of 2-methyl-6-(4-(tetrahydro-2H-pyran-4-ylamino)-1H-pyrazolo[4,3-c]pyridin-3-yl)isonicotinic acid: A microwave vial equipped with a magnetic stirrer was charged with ethyl 2-(1-(4-methoxybenzyl)-4-(tetrahydro-2H-pyran-4-ylamino)-1H-pyrazolo[4,3-c]pyridin-3-yl)-6-methylisonicotinate (200 mg, 0.390 mmol) and TFA (20 mL). The reaction mixture was heated at 150° C. for 2 h under microwave irradiation. After cooling down, the mixture was concentrated under reduced pressure. The residue was purified by reverse-phase prep-HPLC to afford the title compound as a white solid (100 mg, 73%).

Step 3—Synthesis of N,N,2-trimethyl-6-(4-(tetrahydro-2H-pyran-4-ylamino)-1H-pyrazolo[4,3-c]pyridin-3-yl)isonicotinamide: To a 25-mL round bottom flash was charge with 2-methyl-6-(4-(tetrahydro-2H-pyran-4-ylamino)-1H-pyrazolo[4,3-c]pyridin-3-yl)isonicotinic acid (100 mg, 0.280 mmol), dimethylamine (36 mg, 0.45 mmol), HATU (167 mg, 0.450 mmol), DIPEA (0.50 mL), and DMF (20 mL). The reaction mixture was stirred at room temperature for 12 h. After removal of the volatiles, the residue was purified by reverse-phase Combi-flash eluting with 0.3% NH₄HCO₃/CH₃CN (1:3) to afford the title compound as a white solid (86 mg, 81%). ¹H NMR (500 MHz, CD₃OD) δ 8.19 (s, 1H), 7.73 (d, J=6.5 Hz, 1H), 7.30 (s, 1H), 6.70 (d, J=6.5 Hz, 1H), 4.33-4.29 (m, 1H), 4.08-4.05 (m, 2H), 3.65-3.61 (m, 2H), 3.16 (s, 3H), 2.97 (s, 3H), 2.78 (s, 3H), 2.16-2.13 (m, 2H), 1.80-1.72 (m, 2H).

### 3-(pyridin-2-yl)-N-(2,2,2-trifluoroethyl)-1H-pyrazolo[4,3-c]pyridin-4-amine

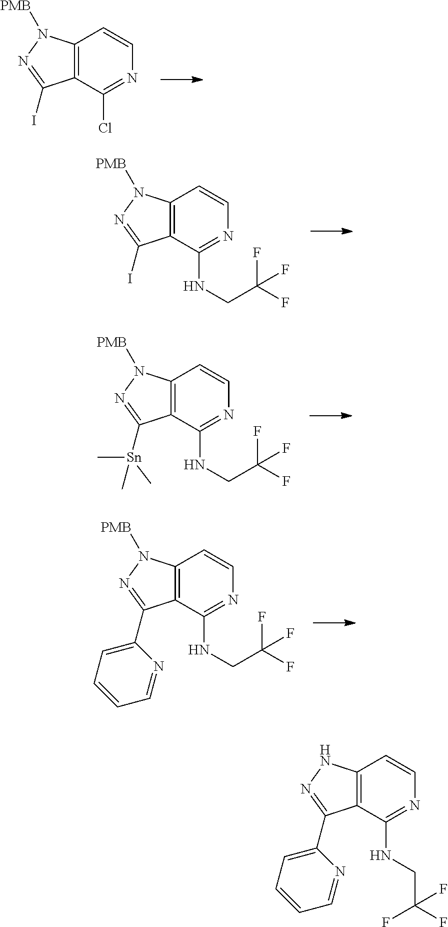

Step 1—Synthesis of 3-iodo-1-(4-methoxybenzyl)-N-(2,2,2-trifluoroethyl)-1H-pyrazolo[4,3-c]pyridin-4-amine: A microwave vial equipped with a magnetic stirrer was charged with 4-chloro-3-iodo-1-(4-methoxybenzyl)-1H-pyrazolo[4,3-c]pyridine (1.0 g, 2.5 mmol), 2,2,2-trifluoroethanamine hydrochloride (1.0 g, 7.5 mmol), DIPEA (2.40 g, 18.6 mmol), and n-BuOH (10 ml). The reaction mixture was heated at 190° C. for 2 h under microwave irradiation. After cooling down, the mixture was concentrated under reduced pressure. The residue was purified by silica gel column chromatography eluting with DCM/petroleum ether/TEA (2:1:0.01) to afford the title compound as a white solid (300 mg, 25%).

Step 2—Synthesis of 1-(4-methoxybenzyl)-N-(2,2,2-trifluoroethyl)-3-(trimethylstannyl)-1H-pyrazolo[4,3-c]pyridin-4-amine: A reaction tube equipped with a magnetic stirrer was charged with 3-iodo-1-(4-methoxybenzyl)-N-(2,2,2-trifluoroethyl)-1H-pyrazolo[4,3-c]pyridin-4-amine (750 mg, 1.62 mmol), 1,1,1,2,2,2-hexamethyldistannane (637 mg, 1.95 mmol), toluene (12 mL), and trans-Pd(PPh₃)₂Cl₂ (39 mg, 0.055 mmol). After three cycles of vacuum/argon flash, the reaction mixture was sealed and heated at 140° C. for 0.5 h. After cooling down, the mixture was filtered. The filtrate was concentrated under reduced pressure to give the title compound, which was used in the next step without further purification.

Step 3—Synthesis of 1-(4-methoxybenzyl)-3-(pyridin-2-yl)-N-(2,2,2-trifluoroethyl)-1H-pyrazolo[4,3-c]pyridin-4-amine: A microwave vial equipped with a magnetic stirrer was charged with N-ethyl-1-(4-methoxybenzyl)-3-(trimethylstannyl)-1H-pyrazolo[4,3-c]pyridin-4-amine (100 mg, 0.200 mmol), 2-bromopyridine (63 mg, 0.40 mmol), LiCl (34 mg, 0.80 mmol), CuI (8.0 mg, 0.040 mmol), Pd(PPh₃)₄ (24 mg, 0.020 mmol), and THF (3 mL). After three cycles of vacuum/argon flash, the reaction mixture was heated at 100° C. for 1 hour under microwave irradiation. After cooling down, the mixture was filtered. The filtrate was concentrated under reduced pressure. The residue was purified by prep-TLC eluting with petroleum ether/ethyl acetate (1:2) to afford the title compound as a yellow solid (76 mg, 92%).

Step 4—Synthesis of 3-(pyridin-2-yl)-N-(2,2,2-trifluoroethyl)-1H-pyrazolo[4,3-c]pyridin-4-amine: A microwave vial equipped with a magnetic stirrer was charged with 1-(4-methoxybenzyl)-3-(pyridin-2-yl)-N-(2,2,2-trifluoroethyl)-1H-pyrazolo[4,3-c]pyridin-4-amine (76.0 mg, 0.184 mole) and TFA (3 mL). The reaction mixture was heated at 120° C. for 2 h under microwave irradiation. After cooling down, the mixture was concentrated under reduced pressure. The residue was purified by prep-HPLC to give the title compound as a yellow solid (8.0 mg, 15%). ¹H NMR (500 MHz, DMSO) 13.41 (br, 1H), 10.95 (t, J=6.0 Hz, 1H), 8.63 (d, J=4.5 Hz, 1H), 8.35 (d, J=8.0 Hz, 1H), 7.99-8.02 (m, 1H), 7.80 (d, J=6.0 Hz, 1H), 7.47-7.49 (m, 1H), 6.78 (d, J=6.0 Hz, 1H), 4.46-4.53 (m, 2H).

### N-(4,4-difluorocyclohexyl)-3-(pyridin-2-yl)-1H-pyrazolo[4,3-c]pyridin-4-amine

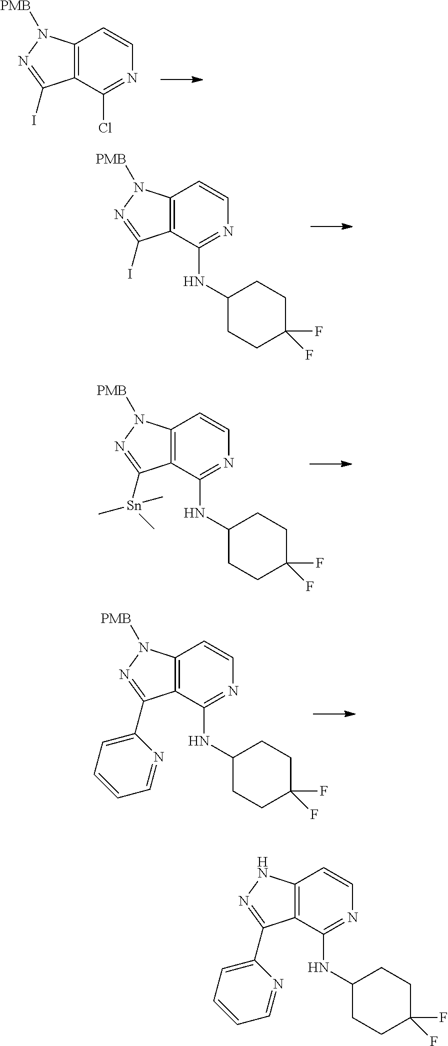

Step 1—Synthesis of N-(4,4-difluorocyclohexyl)-3-iodo-1-(4-methoxybenzyl)-1H-pyrazolo[4,3-c]pyridin-4-amine: A microwave vial equipped with a magnetic stirrer was charged with 4-chloro-3-iodo-1-(4-methoxybenzyl)-1H-pyrazolo[4,3-c]pyridine (400 mg, 1.00 mmol), 4,4-difluorocyclohexanamine hydrochloride (342 mg, 2.00 mmol), DIPEA (336 mg, 3.00 mmol), and n-BuOH (10 mL). The reaction mixture was heated at 170° C. for 2 h under microwave irradiation. After cooling down, the mixture was concentrated under reduced pressure. The residue was purified by silica gel column chromatography eluting with DCM/petroleum ether/TEA (2:1:0.01) to afford the title compound as a grey solid (310 mg, 62%).

Step 2—Synthesis of N-(4,4-difluorocyclohexyl)-1-(4-methoxybenzyl)-3-(trimethylstannyl)-1H-pyrazolo[4,3-c]pyridin-4-amine: A reaction tube equipped with a magnetic stirrer was charged with N-(4,4-difluorocyclohexyl)-3-iodo-1-(4-methoxybenzyl)-1H-pyrazolo[4,3-c]pyridin-4-amine (240 mg, 0.482 mmol), 1,1,1,2,2,2-hexamethyldistannane (315 mg, 0.946 mmol), toluene (12 mL), and trans-Pd(PPh₃)₂Cl₂ (8.5 mg, 0.012 mmol). After three cycles of vacuum/argon flash, the reaction mixture was sealed and heated at 110° C. for 15 h. After cooling down, the mixture was filtered. The filtrate was concentrated under reduced pressure to give the title compound, which was used in the next step without further purification (285 mg, over 100%).

Step 3—Synthesis of N-(4,4-difluorocyclohexyl)-1-(4-methoxybenzyl)-3-(pyridin-2-yl)-1H-pyrazolo[4,3-c]pyridin-4-amine: A microwave vial equipped with a magnetic stirrer was charged with N-(4,4-difluorocyclohexyl)-1-(4-methoxybenzyl)-3-(trimethylstannyl)-1H-pyrazolo[4,3-c]pyridin-4-amine (300 mg, 0.561 mmol), 2-bromopyridine (177 mg, 1.12 mmol), LiCl (94.0 mg, 2.24 mmol), CuI (11 mg, 0.056 mmol), Pd(PPh₃)₄ (63 mg, 0.056 mmol), and THF (3 mL). After three cycles of vacuum/argon flash, the reaction mixture was heated at 100° C. for 1 hour under microwave irradiation. After cooling down, the mixture was filtered. The filtrate was concentrated under reduced pressure. The residue was purified by prep-TLC eluting with petroleum ether/ethyl acetate (1:1) to afford the title compound as a yellow solid (80 mg, 35% yield over two steps).

Step 4—Synthesis of N-(4,4-difluorocyclohexyl)-3-(pyridin-2-yl)-1H-pyrazolo[4,3-c]pyridin-4-amine: A microwave vial equipped with a magnetic stirrer was charged with N-(4,4-difluorocyclohexyl)-1-(4-methoxybenzyl)-3-(pyridin-2-yl)-1H-pyrazolo[4,3-c]pyridin-4-amine (80.0 mg, 0.178 mmol) and TFA (3 mL). The reaction mixture was heated at 120° C. for 2 h under microwave irradiation. After cooling down, the mixture was concentrated under reduced pressure. The residue was purified by reverse-phase prep-HPLC to afford the title compound as a white solid (15 mg, 26%). ¹H NMR (500 MHz, CDCl₃) δ 8.56 (d, J=6.0 Hz, 1H), 8.41 (d, J=8.0 Hz, 1H), 7.91-7.88 (m, 2H), 7.39-7.35 (m, 1H), 6.68-6.66 (m, 1H), 4.43 (br s, 3H), 2.28-2.17 (m, 6H), 1.87-1.79 (m, 2H).

### N-(4,4-difluorocyclohexyl)-3-(pyrimidin-4-yl)-1H-pyrazolo[4,3-c]pyridin-4-amine

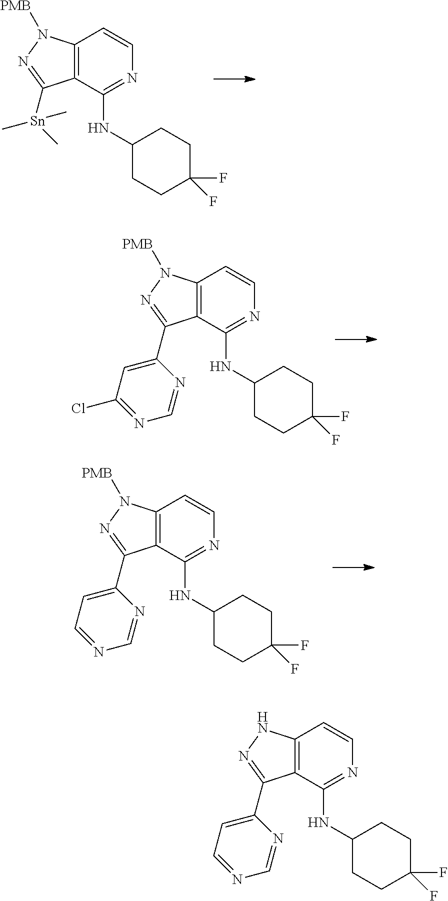

Step 1—Synthesis of 3-(6-chloropyrimidin-4-yl)-N-(4,4-difluorocyclohexyl)-1-(4-methoxybenz-y-1)-1H-pyrazolo[4,3-c]pyridin-4-amine: A microwave vial equipped with a magnetic stirrer was charged with N-(4,4-difluorocyclohexyl)-1-(4-methoxybenzyl)-3-(trimethylstannyl)-1H-pyrazolo[4,3-c]pyridin-4-amine (300 mg, 0.561 mmol), 4,6-dichloropyrimidine (166 mg, 1.12 mmol), LiCl (94.0 mg, 2.24 mmol), CuI (11 mg, 0.056 mmol), Pd(PPh₃)₄ (63 mg, 0.056 mmol), and THF (3 mL). After three cycles of vacuum/argon flash, the reaction mixture was heated at 100° C. for 1 hour under microwave irradiation. After cooling down, the mixture was filtered. The filtrate was concentrated under reduced pressure. The residue was purified by prep-TLC eluting with petroleum ether/ethyl acetate (1:1) to afford the title compound as a yellow solid (110 mg, 41%).

Step 2—Synthesis of N-(4,4-difluorocyclohexyl)-1-(4-methoxybenzyl)-3-(pyrimidin-4-yl)-1H-pyrazolo[4,3-c]pyridin-4-amine: A mixture of 3-(6-chloropyrimidin-4-yl)-N-(4,4-difluorocyclohexyl)-1-(4-methoxybenzyl)-1H-pyrazolo[4,3-c]pyridin-4-amine (110 mg, 0.227 mmol) and Pd/C (20 mg) in methanol (20 mL) was stirred at 50° C. under H₂ for 10 h. The insoluble was filtered off and the filtrate was concentrated under reduced pressure to give the title compound as white oil (90 mg, 88%).

Step 3—Synthesis of N-(4,4-difluorocyclohexyl)-3-(pyrimidin-4-yl)-1H-pyrazolo[4,3-c]pyridin-4-amine: A microwave vial equipped with a magnetic stirrer was charged with N-(4,4-difluorocyclohexyl)-1-(4-methoxybenzyl)-3-(pyrimidin-4-yl)-1H-pyrazolo[4,3-c]pyridin-4-amine (90 mg, 0.20 mmol) and TFA (3 mL). The reaction mixture was heated at 120° C. for 2 h under microwave irradiation. After cooling down, the mixture was concentrated under reduced pressure. The residue was purified by reverse-phase prep-HPLC to afford the title compound as a yellow solid (18 mg, 27%). ¹H NMR (500 MHz, CD₃OD) δ 9.22 (d, J=1.0 Hz, 1H), 8.86 (d, J=6.0 Hz, 1H), 8.42 (d, J=3.5 Hz, 1H), 7.76 (d, J=6.0 Hz, 1H), 6.73 (d, J=6.0 Hz, 1H), 4.17-4.15 (m, 1H), 2.26-2.21 (m, 2H), 2.19-2.09 (m, 2H), 1.87-1.62 (m, 2H), 1.84-1.81 (m, 2H).

### 3-(6-methyl-4-(trifluoromethyl)pyridin-2-yl)-N-(tetrahydro-2H-pyran-4-yl)-1H-pyrazolo[4,3-c]pyridin-4-amine

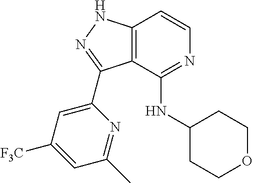 [SMILES: Cc1cc(C(F)(F)F)cc(-c2n[nH]c3ccnc(NC4CCOCC4)c23)n1]

Synthesis of 3-(6-methyl-4-(trifluoromethyl)pyridin-2-yl)-N-(tetrahydro-2H-pyran-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### 3-(2-methyl-6-(trifluoromethyl)pyrimidin-4-yl)-N-(tetrahydro-2H-pyran-4-yl)-1H-pyrazolo[4,3-c]pyridin-4-amine

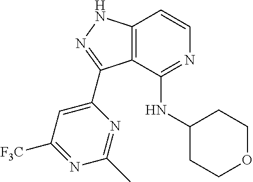 [SMILES: Cc1nc(-c2n[nH]c3ccnc(NC4CCOCC4)c23)cc(C(F)(F)F)n1]

Synthesis of 3-(2-methyl-6-(trifluoromethyl)pyrimidin-4-yl)-N-(tetrahydro-2H-pyran-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-(dicyclopropylmethyl)-3-(6-(trifluoromethyl)pyrimidin-4-yl)-1H-pyrazolo[4,3-c]pyridin-4-amine

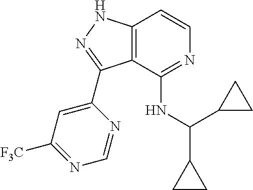 [SMILES: FC(F)(F)c1cc(-c2n[nH]c3ccnc(NC(C4CC4)C4CC4)c23)ncn1]

Synthesis of N-(dicyclopropylmethyl)-3-(6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-(cyclopropylmethyl)-3-(6-(trifluoromethyl)pyrimidin-4-yl)-1H-pyrazolo[4,3-c]pyridin-4-amine

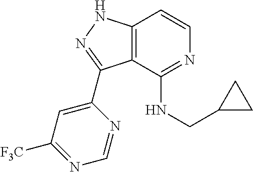 [SMILES: FC(F)(F)c1cc(-c2n[nH]c3ccnc(NCC4CC4)c23)ncn1]

Synthesis of N-(cyclopropylmethyl)-3-(6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-(4,4-difluorocyclohexyl)-3-(6-(trifluoromethyl)pyrimidin-4-yl)-1H-pyrazolo[4,3-c]pyridin-4-amine

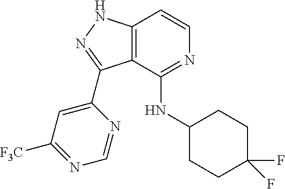 [SMILES: FC1(F)CCC(Nc2nccc3[nH]nc(-c4cc(C(F)(F)F)ncn4)c23)CC1]

Synthesis of N-(4,4-difluorocyclohexyl)-3-(6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-(4,4-difluorocyclohexyl)-3-(2-methyl-6-(trifluoromethyl)pyrimidin-4-yl)-1H-pyrazolo[4,3-c]pyridin-4-amine

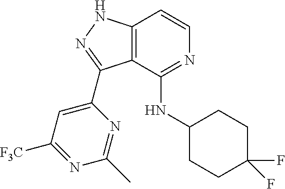 [SMILES: Cc1nc(-c2n[nH]c3ccnc(NC4CCC(F)(F)CC4)c23)cc(C(F)(F)F)n1]

Synthesis of N-(4,4-difluorocyclohexyl)-3-(2-methyl-6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-((2R,6S)-2,6-dimethyltetrahydro-2H-pyran-4-yl)-3-(6-(trifluoromethyl)pyrimidin-4-yl)-1H-pyrazolo[4,3-c]pyridin-4-amine

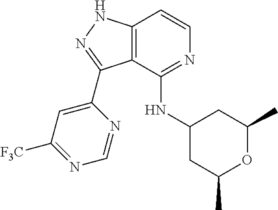 [SMILES: C[C@@H]1CC(Nc2nccc3[nH]nc(-c4cc(C(F)(F)F)ncn4)c23)C[C@H](C)O1]

Synthesis of N-((2R,6S)-2,6-dimethyltetrahydro-2H-pyran-4-yl)-3-(6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-(8-oxabicyclo[3.2.1]octan-3-yl)-3-(6-(trifluoromethyl)pyrimidin-4-yl)-1H-pyrazolo[4,3-c]pyridin-4-amine

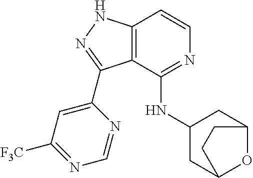 [SMILES: FC(F)(F)c1cc(-c2n[nH]c3ccnc(NC4CC5CCC(C4)O5)c23)ncn1]

Synthesis of N-(8-oxabicyclo[3.2.1]octan-3-yl)-3-(6-(trifluoromethyl)pyrimidin-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-isopropyl-3-(4-(methylsulfonyl)pyridin-2-yl)-1H-pyrazolo[4,3-c]pyridin-4-amine

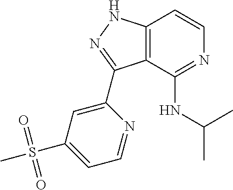 [SMILES: CC(C)Nc1nccc2[nH]nc(-c3cc(S(C)(=O)=O)ccn3)c12]

Synthesis of N-isopropyl-3-(4-(methylsulfonyl)pyridin-2-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### 3-(4-(methylsulfonyl)pyridin-2-yl)-N-(tetrahydro-2H-pyran-4-yl)-1H-pyrazolo[4,3-c]pyridin-4-amine

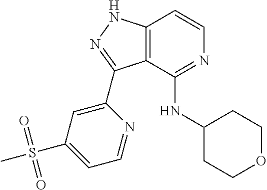 [SMILES: CS(=O)(=O)c1ccnc(-c2n[nH]c3ccnc(NC4CCOCC4)c23)c1]

Synthesis of 3-(4-(methylsulfonyl)pyridin-2-yl)-N-(tetrahydro-2H-pyran-4-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### N-isopropyl-3-(4-(isopropylsulfonyl)pyridin-2-yl)-1H-pyrazolo[4,3-c]pyridin-4-amine

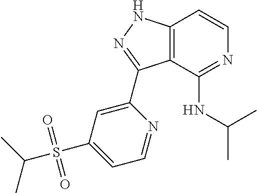 [SMILES: CC(C)Nc1nccc2[nH]nc(-c3cc(S(=O)(=O)C(C)C)ccn3)c12]

Synthesis of N-isopropyl-3-(4-(isopropylsulfonyl)pyridin-2-yl)-1H-pyrazolo[4,3-c]pyridin-4-amine: The compound was prepared according to the general procedure described in the synthesis of N-isopropyl-3-(pyridin-2-yl)-1H-pyrazolo[4,3-c]pyridin-4-amine to give the title compound.

### 3-(5-cyclopropyl-4-methyl-4H-1,2,4-triazol-3-yl)-N-isopropyl-1H-pyrazolo[4,3-c]pyridin-4-amine and 3-(5-cyclopropyl-1-methyl-1H-1,2,4-triazol-3-yl)-N-isopropyl-1H-pyrazolo[4,3-c]pyridin-4-amine

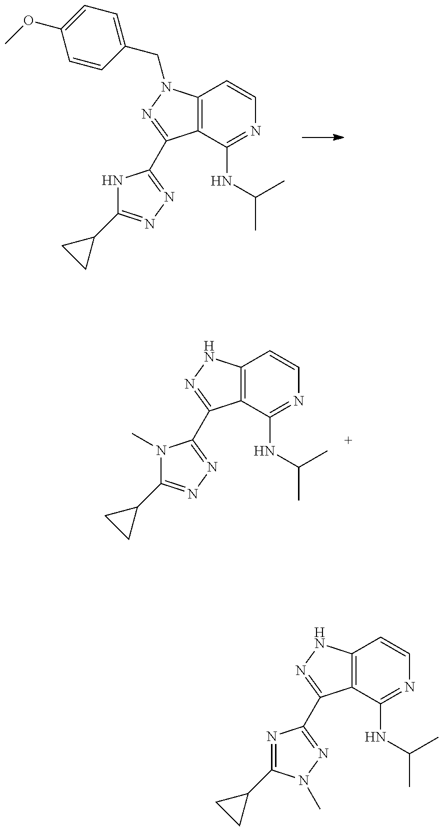 [SMILES: COc1ccc(Cn2nc(-c3nnc(C4CC4)[nH]3)c3c(NC(C)C)nccc32)cc1 ; CC(C)Nc1nccc2[nH]nc(-c3nnc(C4CC4)n3C)c12 ; CC(C)Nc1nccc2[nH]nc(-c3nc(C4CC4)n(C)n3)c12]

Synthesis of 3-(5-cyclopropyl-4-methyl-4H-1,2,4-triazol-3-yl)-N-isopropyl-1H-pyrazolo[4,3-c]pyridin-4-amine and 3-(5-cyclopropyl-1-methyl-1H-1,2,4-triazol-3-yl)-N-isopropyl-1H-pyrazolo[4,3-c]pyridin-4-amine: To a vial was added 3-(5-cyclopropyl-4H-1,2,4-triazol-3-yl)-N-isopropyl-1-(4-methoxybenzyl)-1H-pyrazolo[4,3-c]pyridin-4-amine (48.5 mg; 0.120 mmol), cesium carbonate (58.7 mg; 0.180 mmol) and dimethylformamide (1.8 mL, 22.8 mmol). Iodomethane, (0.011 mL, 0.180 mmol) was added, the vial was capped and the reaction was heated to 90° C. for 2 h. The reaction mixture was diluted with DCM and water and the layers were separated. The aqueous layer was extracted with DCM (2×), the combined organics were dried over sodium sulfate, filtered and concentrated in vacuo. The crude residue was purified by column chromatography (0-100% EtOAc in DCM). Recovered two regioisomers (20.0 mg, 40%; 12.9 mg, 26%). The separated regioisomers were deprotected according to the procedure described for 3-(5-isopropyl-4H-1,2,4-triazol-3-yl)-N-(tetrahydro-2H-pyran-4-yl)-1H-pyrazolo[4,3-c]pyridin-4-amine to give the title, putatively assigned regioisomeric compounds. ¹H NMR (400 MHz, DMSO) δ 13.21 (s, 1H), 9.09 (d, J=7.1 Hz, 1H), 7.71 (d, J=6.0 Hz, 1H), 6.57 (d, J=6.0 Hz, 1H), 4.40-4.30 (m, 1H), 3.98 (s, 3H), 2.32-2.23 (m, 1H), 1.27 (s, 3H), 1.26 (s, 3H), 1.22-1.13 (m, 2H), 1.06-0.99 (m, 2H) and δ 13.60 (s, 1H), 9.43 (d, J=7.2 Hz, 1H), 7.78 (d, J=6.0 Hz, 1H), 6.64 (d, J=6.0 Hz, 1H), 4.44-4.30 (m, 1H), 2.13-2.03 (m, 1H), 1.27 (d, J=6.5 Hz, 6H), 1.08-0.99 (m, 2H), 0.92-0.85 (m, 2H).

### 3-(5-ethyl-1-methyl-1H-pyrazol-3-yl)-N-(tetrahydro-2H-pyran-4-yl)-1H-pyrazolo[4,3-c]pyridin-4-amine

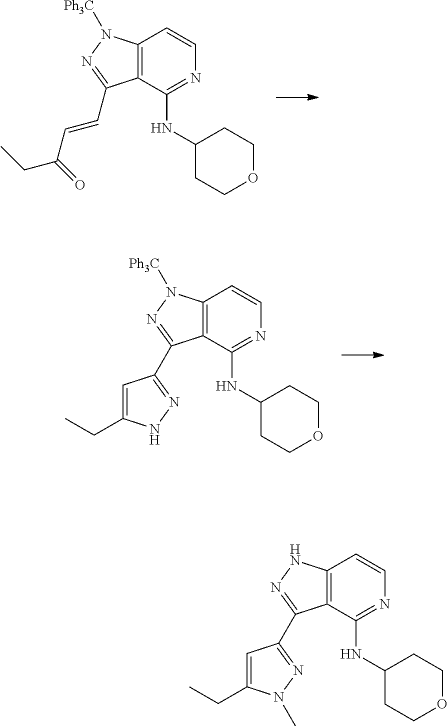

Step 1—Synthesis of 3-(5-ethyl-1H-pyrazol-3-yl)-N-(tetrahydro-2H-pyran-4-yl)-1-trityl-1H-pyrazolo[4,3-c]pyridin-4-amine: To a microwave vial was added 1-(4-((tetrahydro-2H-pyran-4-yl)amino)-1-trityl-1H-pyrazolo[4,3-c]pyridin-3-yl)pent-1-en-3-one (0.114 g, 0.210 mmol) followed by N,N-dimethylformamide (2.43 mL, 31.4 mmol). Hydrazine hydrate (0.0409 mL, 0.840 mmol) was added and the reaction was heated to 180° C. for 10 mins under microwave heating. The reaction mixture was poured into water and extracted with EtOAc (3×), dried over sodium sulfate, filtered and concentrated in vacuo. The crude residue was then dissolved dichloromethane (1 mL, 15 mmol) and 2,3-dichloro-5,6-dicyano-1,4-benzoquinone (0.143 g, 0.630 mmol) was added. The reaction was stirred overnight at room temp. The reaction mixture was filtered through Celite and concentrated in vacuo. The crude residue was flashed with 0-100% EtOAc in DCM to give the title compound (117 mg, 98%).

Step 2—Synthesis of 3-(5-ethyl-1-methyl-1H-pyrazol-3-yl)-N-(tetrahydro-2H-pyran-4-yl)-1H-pyrazolo[4,3-c]pyridin-4-amine: To a solution of 3-(5-ethyl-1H-pyrazol-3-yl)-N-(tetrahydro-2H-pyran-4-yl)-1-trityl-1H-pyrazolo[4,3-c]pyridin-4-amine (0.114 g, 0.206 mmol) in N,N-dimethylformamide (3.0 mL, 39 mmol) at rt was added cesium carbonate (0.100 g, 0.308 mmol) and methyl iodide (0.0192 mL, 0.308 mmol). The solution was stirred for 2 h at 60° C. The reaction mixture was diluted with EtOAc and water, the layers were separated, and the aqueous layer was extracted with EtOAc (2×). The combined organics extracts were dried over sodium sulfate, filtered and concentrated in vacuo. The residue was purified by column chromatography (0-100% EtOAc in DCM). The combined product fractions were concentrated in vacuo and dissolved in dichloromethane (3.00 mL, 46.8 mmol). Triethylsilane (0.131 mL, 0.823 mmol) and trifluoroacetic acid (3.00 mL, 38.9 mmol) were added and the reaction was stirred at room temperature for 10 mins. The reaction was concentrated in vacuo and the crude residue was purified by reverse-phase HPLC to give the title compound (16.6 mg, 25%). ¹H NMR (400 MHz, DMSO) δ 12.99 (s, 1H), 9.44 (d, J=6.6 Hz, 1H), 7.69 (d, J=6.0 Hz, 1H), 6.59-6.54 (m, 2H), 4.28-4.17 (m, 1H), 3.96-3.88 (m, 2H), 3.85 (s, 3H), 3.55-3.45 (m, 2H), 2.69 (q, J=7.5 Hz, 2H), 2.14-2.02 (m, 2H), 1.64-1.51 (m, 2H), 1.26 (t, J=7.6 Hz, 3H).

### 3-(5-ethyl-1-methyl-pyrazol-3-yl)-N-isopropyl-1-[(4-methoxyphenyl)methyl]pyrazolo[4,3-c]pyridin-4-amine

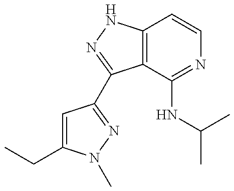 [SMILES: CCc1cc(-c2n[nH]c3ccnc(NC(C)C)c23)nn1C]

Synthesis of 3-(5-ethyl-1-methyl-pyrazol-3-yl)-N-isopropyl-1-[(4-methoxyphenyl)methyl]pyrazolo[4,3-c]pyridin-4-amine: The compound was prepared according to the general procedure described for 3-(5-ethyl-1-methyl-1H-pyrazol-3-yl)-N-(tetrahydro-2H-pyran-4-yl)-1H-pyrazolo[4,3-c]pyridin-4-amine. ¹H NMR (400 MHz, DMSO) δ 12.96 (s, 1H), 9.39 (d, J=6.6 Hz, 1H), 7.69 (d, J=6.0 Hz, 1H), 6.56 (s, 1H), 6.53 (d, J=6.0 Hz, 1H), 4.25 (hept, J=6.5 Hz, 1H), 3.84 (s, 3H), 2.69 (q, J=7.5 Hz, 2H), 1.30 (d, J=6.5 Hz, 6H), 1.25 (t, J=7.5 Hz, 3H).

### 3-(1-isopropyl-1H-imidazol-4-yl)-N-(tetrahydro-2H-pyran-4-yl)-1H-pyrazolo[4,3-c]pyridin-4-amine

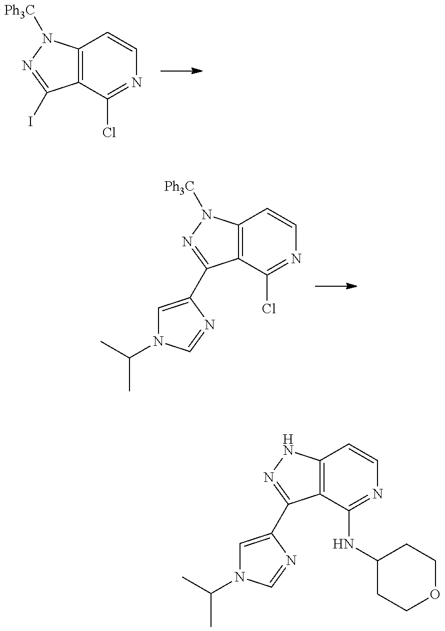

Step 1—Synthesis of 4-chloro-3-(1-isopropyl-1H-imiazol-4-yl)-1-trityl-1H-pyrazolo[4,3-c]pyridine: To a microwave vial was added 4-chloro-3-iodo-1-trityl-1H-pyrazolo[4,3-c]pyridine (0.44 g, 0.00085 mol), 1-isopropyl-4-(tributylstannyl)-1H-imidazole (0.338 g, 0.847 mmol), [1,1′-bis(diphenylphosphino)ferrocene]dichloropalladium(II) (0.069 g, 0.085 mmol), copper(I) thiophene-2-carboxylate (0.242 g, 1.27 mmol), cesium fluoride (0.26 g, 1.70 mmol). Toluene (17.9 mL, 168 mmol) was added and nitrogen was bubbled through the mixture for 2 mins. The reaction mixture was heated to 100° C. under microwave irradiation for 10 mins then filtered through Celite eluting with DCM and concentrated in vacuo. The crude mixture was used without further purification in the next step.

Step 2—Synthesis of 3-(1-isopropyl-1H-imidazol-4-yl)-N-(tetrahydro-2H-pyran-4-yl)-1H-pyrazolo[4,3-c]pyridin-4-amine: To a microwave vial was added crude 4-chloro-3-(1-isopropyl-1H-imidazol-4-yl)-1-trityl-1H-pyrazolo[4,3-c]pyridine (0.064 g, 0.13 mmol), 4-aminotetrahydropyran (0.0154 mL, 0.152 mmol), BrettPhos palladium pre-catalyst (0.0101 g, 0.0127 mmol), BrettPhos (0.00682 g, 0.0127 mmol) and sodium tert-butoxide (0.0293 g, 0.305 mmol). The vial was purged with nitrogen and 1,4-dioxane (1.4 mL, 18 mmol) was added. Nitrogen was then bubbled through the reaction mixture for 5 mins then the reaction mixture was heated in an oil bath to 110° C. overnight. The reaction was then filtered through Celite, eluting with DCM, concentrated in vacuo and purified by column chromatography (0-10% DCM in MeOH). The combined product fractions were concentrated in vacuo and dissolved in dichloromethane (1.8 mL, 28 mmol). Triethylsilane (0.0811 mL, 0.508 mmol) and trifluoroacetic acid (1.8 mL, 23 mmol) were added and the reaction was stirred at room temperature for 15 mins. The reaction was concentrated in vacuo and the crude residue was purified by reverse-phase HPLC to give the title compound (4.6 mg, 11% over two steps). ¹H NMR (400 MHz, DMSO) δ 12.83 (s, 1H), 10.42 (d, J=6.8 Hz, 1H), 7.99 (s, 1H), 7.77 (s, 1H), 7.66 (d, J=6.0 Hz, 1H), 6.52 (d, J=5.9 Hz, 1H), 4.58-4.43 (m, 1H), 4.34-4.22 (m, 1H), 3.95-3.87 (m, 2H), 3.58-3.47 (m, 2H), 2.06-1.95 (m, 2H), 1.63-1.51 (m, 2H), 1.48 (d, J=6.7 Hz, 6H).

### N-isopropyl-3-(1-isopropyl-1H-1,2,3-triazol-4-yl)-1H-pyrazolo[4,3-c]pyridin-4-amine

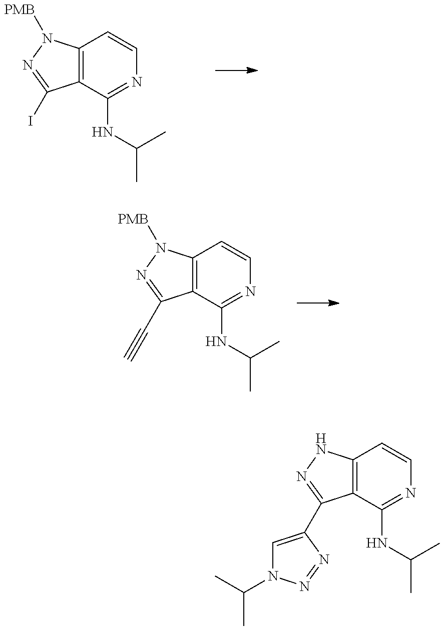

Step 1—Synthesis of 3-ethynyl-N-isopropyl-1-(4-methoxybenzyl)-1H-pyrazolo[4,3-c]pyridin-4-amine: To a microwave vial was added 3-iodo-N-isopropyl-1-(4-methoxybenzyl)-1H-pyrazolo[4,3-c]pyridin-4-amine (0.350 g, 0.829 mmol), bis(triphenylphosphine)palladium(II) chloride (0.0451 g, 0.0643 mmol), copper(I) iodide (0.0122 g, 0.0643 mmol). The vial was purged with nitrogen and acetonitrile (2.2 mL, 43 mmol), triethylamine (0.470 mL, 3.37 mol), and (trimethylsilyl)acetylene (0.136 mL, 0.964 mmol) were added sequentially. The vial was sealed and stirred at rt for 1 h. The reaction mixture was filtered through Celite, eluting with DCM and concentrated in vacuo. The residue was purified by column chromatography (0-5% EtOAc in DCM). The combined product fractions were concentrated in vacuo and dissolved in methanol (60.2 mL, 149 mmol). Potassium carbonate (0.358 g, 2.59 mmol) was added and the reaction mixture was stirred at rt for 30 mins. The mixture was concentrated in vacuo and redissolved in dichloromethane and water. The layers were separated and the aqueous layer was extracted with DCM (2×). The combined organic extracts were dried over sodium sulfate, filtered and concentrated in vacuo to give the title compound which was used directly in the next step.

Step 2—Synthesis of N-isopropyl-3-(1-isopropyl-1H-1,2,3-triazol-4-yl)-1H-pyrazolo[4,3-c]pyridin-4-amine: To a vial was added 3-ethynyl-N-isopropyl-1-(4-methoxybenzyl)-1H-pyrazolo[4,3-c]pyridin-4-amine (0.0907 g, 0.283 mmol), sodium azide (0.0221 g, 0.340 mmol), and copper(I) iodide (0.00539 g, 0.0283 mmol). Tert-butyl alcohol (0.1 mL, 1 mmol), water (0.56 mL, 31 mmol) and isopropyl iodide (0.0340 mL, 0.340 mmol) were added and the reaction was heated to 100° C. in an oil bath overnight. The reaction mixture was poured into sat. aq. ammonium chloride and extracted with DCM (3×). The combined organic extracts were dried over sodium sulfate, filtered and concentrated in vacuo. The residue was purified by column chromatography (0-100% EtOAc in DCM). The combined product fractions were concentrated in vacuo and dissolved in methylene chloride (4.00 mL, 62.4 mmol). Trifluoromethanesulfonic acid (0.15 mL, 1.7 mmol) was added and the reaction mixture was stirred for 1 h at room temp. The reaction mixture was diluted with DCM and water and made basic by slow addition of sat. aq. sodium bicarbonate. The organic layer was separated and the aqueous layer was extracted with CHCl₃: ⁱPrOH (5:1) (3×). The combined organic extracts were dried over sodium sulfate, filtered and concentrated in vacuo. The crude residue was purified by reverse-phase HPLC to give the title compound (25.9 mg, 32% over two steps). ¹H NMR (400 MHz, DMSO) δ 13.18 (s, 1H), 9.30 (d, J=6.6 Hz, 1H), 8.70 (s, 1H), 7.74 (d, J=6.0 Hz, 1H), 6.59 (d, J=6.0 Hz, 1H), 4.99-4.88 (m, 1H), 4.38-4.25 (m, 1H), 1.58 (d, J=6.7 Hz, 6H), 1.28 (d, J=6.5 Hz, 6H).

### N-isopropyl-3-(1-methyl-1H-1,2,3-triazol-4-yl)-1H-pyrazolo[4,3-c]pyridin-4-amine

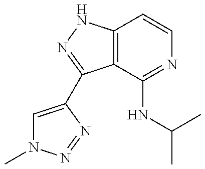 [SMILES: CC(C)Nc1nccc2[nH]nc(-c3cn(C)nn3)c12]

Synthesis of N-isopropyl-3-(1-methyl-1H-1,2,3-triazol-4-yl)-1H-pyrazolo[4,3-c]pyridin-4-amine: The compound was prepared according to the general procedure described for N-isopropyl-3-(1-isopropyl-1H-1,2,3-triazol-4-yl)-1H-pyrazolo[4,3-c]pyridin-4-amine. ¹H NMR (400 MHz, DMSO) δ 13.16 (s, 1H), 9.22 (d, J=6.8 Hz, 1H), 8.57 (s, 1H), 7.74 (d, J=6.0 Hz, 1H), 6.59 (d, J=6.0 Hz, 1H), 4.37-4.25 (m, 1H), 4.16 (s, 3H), 1.27 (d, J=6.5 Hz, 6H).

### N-ethyl-3-(1-isopropyl-1H-1,2,3-triazol-4-yl)-1H-pyrazolo[4,3-c]pyridin-4-amine

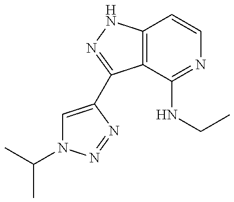 [SMILES: CCNc1nccc2[nH]nc(-c3cn(C(C)C)nn3)c12]

Synthesis of N-ethyl-3-(1-isopropyl-1H-1,2,3-triazol-4-yl)-1H-pyrazolo[4,3-c]pyridin-4-amine: The compound was prepared according to the general procedure described for N-isopropyl-3-(1-isopropyl-1H-1,2,3-triazol-4-yl)-1H-pyrazolo[4,3-c]pyridin-4-amine. ¹H NMR (400 MHz, DMSO) δ 13.19 (s, 1H), 9.28 (t, J=4.9 Hz, 1H), 8.71 (s, 1H), 7.74 (d, J=6.0 Hz, 1H), 6.60 (d, J=6.0 Hz, 1H), 4.93 (hept, J=6.8 Hz, 1H), 3.59-3.47 (m, 2H), 1.58 (d, J=6.7 Hz, 6H), 1.26 (t, J=7.2 Hz, 3H).

### N-ethyl-3-(1-methyl-1H-1,2,3-triazol-4-yl)-1H-pyrazolo[4,3-c]pyridin-4-amine

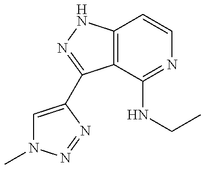 [SMILES: CCNc1nccc2[nH]nc(-c3cn(C)nn3)c12]

Synthesis of N-ethyl-3-(1-methyl-1H-1,2,3-triazol-4-yl)-1H-pyrazolo[4,3-c]pyridin-4-amine: The compound was prepared according to the general procedure described for N-isopropyl-3-(1-isopropyl-1H-1,2,3-triazol-4-yl)-1H-pyrazolo[4,3-c]pyridin-4-amine. ¹H NMR (400 MHz, DMSO) δ 13.18 (s, 1H), 9.20 (t, J=4.9 Hz, 1H), 8.58 (s, 1H), 7.74 (d, J=6.0 Hz, 1H), 6.61 (d, J=6.0 Hz, 1H), 4.16 (s, 3H), 3.59-3.48 (m, 2H), 1.26 (t, J=7.2 Hz, 3H).

### N-ethyl-3-[1-(2-methoxyethyl)triazol-4-yl]-1H-pyrazolo[4,3-c]pyridin-4-amine

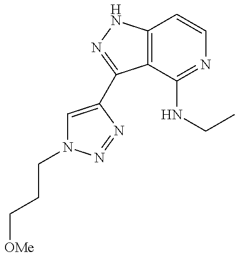 [SMILES: CCNc1nccc2[nH]nc(-c3cn(CCCOC)nn3)c12]

Synthesis of N-ethyl-3-[1-(2-methoxyethyl)triazol-4-yl]-1H-pyrazolo[4,3-c]pyridin-4-amine: The compound was prepared according to the general procedure described for N-isopropyl-3-(1-isopropyl-1H-1,2,3-triazol-4-yl)-1H-pyrazolo[4,3-c]pyridin-4-amine. ¹H NMR (400 MHz, DMSO) δ 13.20 (s, 1H), 9.22 (t, J=4.7 Hz, 1H), 8.59 (s, 1H), 7.75 (d, J=6.0 Hz, 1H), 6.61 (d, J=6.0 Hz, 1H), 4.65 (t, J=5.2 Hz, 2H), 3.83 (t, J=5.2 Hz, 2H), 3.58-3.49 (m, 2H), 3.28 (s, 3H), 1.26 (t, J=7.2 Hz, 3H).

### N-isopropyl-1-[(4-methoxyphenyl)methyl]-3-[4-(trifluoromethyl)pyrazol-1-yl]pyrazolo[4,3-c]pyridin-4-amine

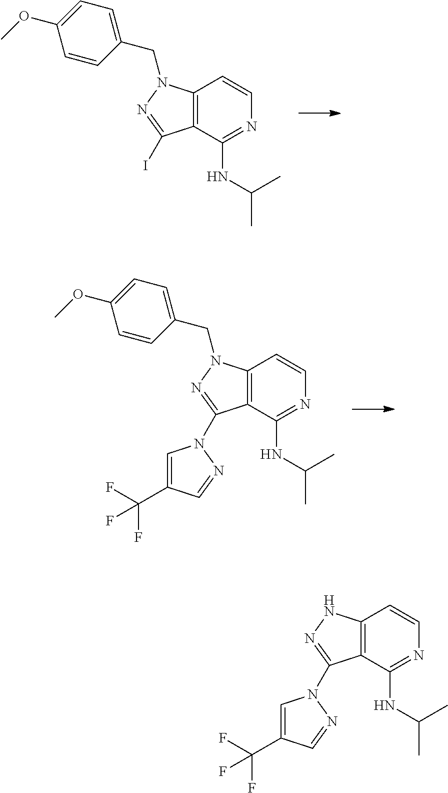

Step 1—Synthesis of N-isopropyl-1-(4-methoxybenzyl)-3-(4-(trifluoromethyl)-1H-pyrazol-1-yl)-1H-pyrazolo[4,3-c]pyridin-4-amine: To a vial was added 3-iodo-N-isopropyl-1-[(4-methoxyphenyl)methyl]pyrazolo[4,3-c]pyridin-4-amine (100 mg, 0.237 mmol), 4-(trifluoromethyl)-1H-pyrazole (30.6 mg, 0.225 mmol), copper(I) iodide (11.3 mg, 0.059 mmol), potassium carbonate (57.8 mg, 0.414 mmol). The vial was purged with nitrogen and toluene (0.47 mL, 4.45 mmol) and trans-N,N′-dimethylcyclohexane-1,2-diamine, (0.02 mL, 0.12 mmol) were added. The vial was sealed and heated to 110° C. for 24 h. The reaction mixture was filtered through Celite, eluting with DCM and concentrated in vacuo. The crude residue was purified by column chromatography (0-20% EtOAc in DCM) to give the title compound which was used directly in the next step.

Step 2—Synthesis of N-isopropyl-1-[(4-methoxyphenyl)methyl]-3-[4-(trifluoromethyl)pyrazol-1-yl]pyrazolo[4,3-c]pyridin-4-amine: To a solution of N-isopropyl-1-(4-methoxybenzyl)-3-(4-(trifluoromethyl)-1H-pyrazol-1-yl)-1H-pyrazolo[4,3-c]pyridin-4-amine (15.2 mg, 0.035 mmol) in dichloromethane (1.0 mL; 16 mmol) was added trifluoromethanesulfonic acid (0.032 mL, 0.35 mmol). The reaction mixture was stirred at rt for 30 mins then diluted with DCM and water and made basic by slow addition of sodium bicarbonate. The layers were separated and the aqueous was extracted with DCM (2×). The combined organic extracts were dried over sodium sulfate, filtered, concentrated in vacuo. The crude residue was purified by reverse-phase HPLC to give the title compound (mg, 9% over two steps). ¹H NMR (400 MHz, DMSO) δ 13.30 (s, 1H), 9.13 (s, 1H), 8.48 (s, 1H), 8.32 (d, J=6.8 Hz, 1H), 7.80 (d, J=6.0 Hz, 1H), 6.64 (d, J=6.0 Hz, 1H), 4.33-4.20 (m, 1H), 1.24 (d, J=6.5 Hz, 6H).

The person skilled in the art will appreciate that it may be necessary or desirable to modify the conditions for each specific compound, such as changing the number or equivalents of reagents, changing the solvent, changing the temperature, changing the reaction time. In the case of palladium catalysed reactions, using a different palladium salt, ligand or base. It may also be necessary or desirable to employ different work-up or purification techniques.

The compounds described in the above examples, as well as additional compounds made using these procedures, are shown in Table 1, together with their LRRK2 affinity values.

Various modifications and variations of the described aspects of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

e1: Structures and K_{I} (μM) values for selected compounds according to the invention

| | Name | Structure | K_{I} (μM) |
|---|---|---|---|
| 1 | 3-(5-ethyl-1-methyl-1H- pyrazol-3-yl)-N-(tetrahydro- 2H-pyran-4-yl)-1H- pyrazolo[4,3-c]pyridin-4- amine | 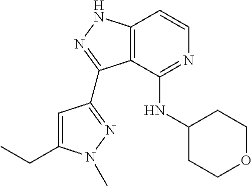 [SMILES: CCc1cc(-c2n[nH]c3ccnc(NC4CCOCC4)c23)nn1C] | 0.0005 |
| 2 | N-isopropyl-3-(4- morpholinopyrimidin-2-yl)- 1H-pyrazolo[4,3-c]pyridin-4- amine | 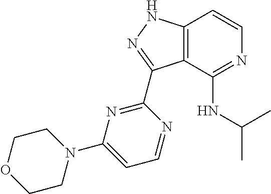 [SMILES: CC(C)Nc1nccc2[nH]nc(-c3nccc(N4CCOCC4)n3)c12] | 0.0785 |
| 3 | N-isopropyl-3-(4- morpholinopyridin-2-yl)-1H- pyrazolo[4,3-c]pyridin-4- amine | 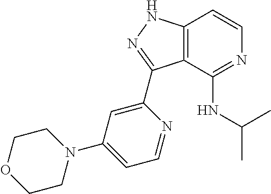 [SMILES: CC(C)Nc1nccc2[nH]nc(-c3cc(N4CCOCC4)ccn3)c12] | 0.0016 |
| 4 | N-isopropyl-3-(6- morpholinopyrimidin-4-yl)- 1H-pyrazolo[4,3-c]pyridin-4- amine | 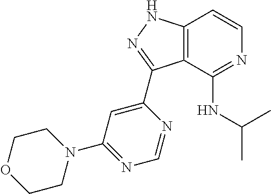 [SMILES: CC(C)Nc1nccc2[nH]nc(-c3cc(N4CCOCC4)ncn3)c12] | 0.0011 |
| 5 | 3-(1-isopropyl-1H-1,2,3- triazol-4-yl)-N-(tetrahydro- 2H-pyran-4-yl)-1H- pyrazolo[4,3-c]pyridin-4- amine | 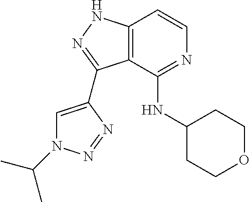 [SMILES: CC(C)n1cc(-c2n[nH]c3ccnc(NC4CCOCC4)c23)nn1] | 0.0017 |
| 6 | 3-(1-isopropyl-1H-imidazol- 4-yl)-N-(tetrahydro-2H- pyran-4-yl)-1H-pyrazolo[4,3- c]pyridin-4-amine | 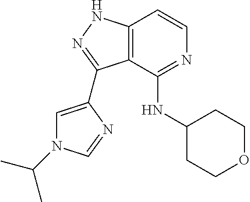 [SMILES: CC(C)n1cnc(-c2n[nH]c3ccnc(NC4CCOCC4)c23)c1] | 0.0033 |
| 7 | N-isopropyl-3-(1-isopropyl- 1H-1,2,3-triazol-4-yl)-1H- pyrazolo[4,3-c]pyridin-4- amine | 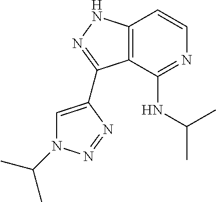 [SMILES: CC(C)Nc1nccc2[nH]nc(-c3cn(C(C)C)nn3)c12] | 0.0044 |
| 8 | N-isopropyl-3-(1-methyl- 1H-1,2,3-triazol-4-yl)-1H- pyrazolo[4,3-c]pyridin-4- amine | 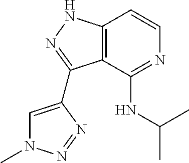 [SMILES: CC(C)Nc1nccc2[nH]nc(-c3cn(C)nn3)c12] | 0.0052 |
| 9 | N-ethyl-3-(1-isopropyl-1H- 1,2,3-triazol-4-yl)-1H- pyrazolo[4,3-c]pyridin-4- amine | 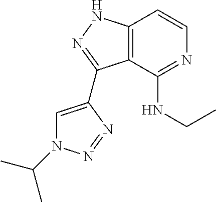 [SMILES: CCNc1nccc2[nH]nc(-c3cn(C(C)C)nn3)c12] | 0.0131 |
| 10 | N-ethyl-3-(1-methyl-1H- 1,2,3-triazol-4-yl)-1H- pyrazolo[4,3-c]pyridin-4- amine | 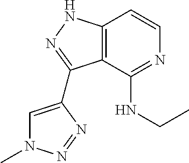 [SMILES: CCNc1nccc2[nH]nc(-c3cn(C)nn3)c12] | 0.0145 |
| 11 | 3-(5-ethyl-1-methyl-1H- pyrazol-3-yl)-N-isopropyl- 1H-pyrazolo[4,3-c]pyridin-4- amine | 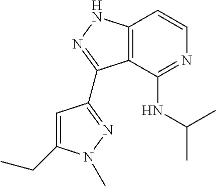 [SMILES: CCc1cc(-c2n[nH]c3ccnc(NC(C)C)c23)nn1C] | 0.0035 |
| 12 | N-ethyl-3-(1-methyl-1H- imidazol-4-yl)-1H- pyrazolo[4,3-c]pyridin-4- amine | 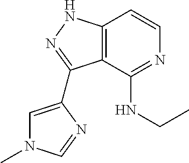 [SMILES: CCNc1nccc2[nH]nc(-c3cn(C)cn3)c12] | 0.0337 |
| 13 | N-isopropyl-3-(pyridin-2- yl)-1H-pyrazolo[4,3- c]pyridin-4-amine | 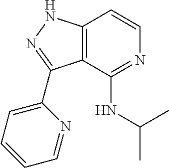 [SMILES: CC(C)Nc1nccc2[nH]nc(-c3ccccn3)c12] | 0.011 |
| 14 | N-ethyl-3-(1-(2- methoxyethyl)-1H-1,2,3- triazol-4-yl)-1H- pyrazolo[4,3-c]pyridin-4- amine | 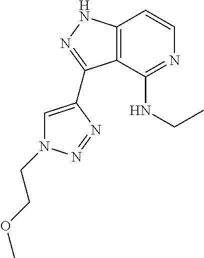 [SMILES: CCNc1nccc2[nH]nc(-c3cn(CCOC)nn3)c12] | 0.0225 |
| 15 | N-isopropyl-3-(pyrimidin-4- yl)-1H-pyrazolo[4,3- c]pyridin-4-amine | 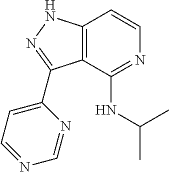 [SMILES: CC(C)Nc1nccc2[nH]nc(-c3ccncn3)c12] | 0.0209 |
| 16 | N-ethyl-3-(pyridin-2-yl)-1H- pyrazolo[4,3-c]pyridin-4- amine | 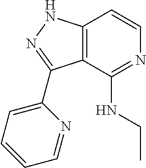 [SMILES: CCNc1nccc2[nH]nc(-c3ccccn3)c12] | 0.0664 |
| 17 | 3-(pyridin-2-yl)-N- (tetrahydro-2H-pyran-4-yl)- 1H-pyrazolo[4,3-c]pyridin-4- amine | 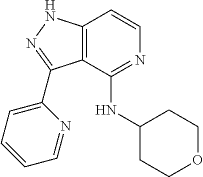 [SMILES: c1ccc(-c2n[nH]c3ccnc(NC4CCOCC4)c23)nc1] | 0.0031 |
| 18 | 3-(pyrimidin-4-yl)-N- (tetrahydro-2H-pyran-4-yl)- 1H-pyrazolo[4,3-c]pyridin-4- amine | 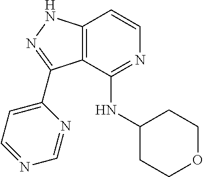 [SMILES: c1cc(-c2n[nH]c3ccnc(NC4CCOCC4)c23)ncn1] | 0.0060 |
| 19 | N-ethyl-3-(pyrimidin-4-yl)- 1H-pyrazolo[4,3-c]pyridin-4- amine | 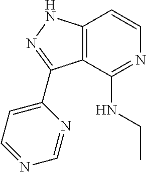 [SMILES: CCNc1nccc2[nH]nc(-c3ccncn3)c12] | 0.152 |
| 20 | N-isopropyl-3-(4- (trifluoromethyl)pyridin-2- yl)-1H-pyrazolo[4,3- c]pyridin-4-amine | 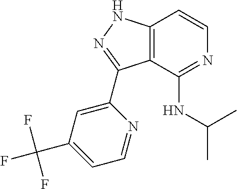 [SMILES: CC(C)Nc1nccc2[nH]nc(-c3cc(C(F)(F)F)ccn3)c12] | 0.0016 |
| 21 | N-ethyl-3-(4- (trifluoromethyl)pyridin-2- yl)-1H-pyrazolo[4,3- c]pyridin-4-amine | 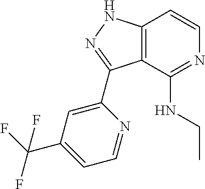 [SMILES: CCNc1nccc2[nH]nc(-c3cc(C(F)(F)F)ccn3)c12] | 0.0094 |
| 22 | N-(tetrahydro-2H-pyran-4- yl)-3-(6- (trifluoromethyl)pyrimidin-4- yl)-1H-pyrazolo[4,3- c]pyridin-4-amine | 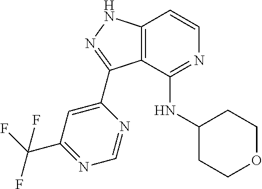 [SMILES: FC(F)(F)c1cc(-c2n[nH]c3ccnc(NC4CCOCC4)c23)ncn1] | 0.0029 |
| 23 | N-(tetrahydro-2H-pyran-4- yl)-3-(4- (trifluoromethyl)pyridin-2- yl)-1H-pyrazolo[4,3- c]pyridin-4-amine | 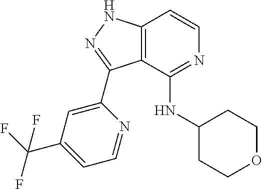 [SMILES: FC(F)(F)c1ccnc(-c2n[nH]c3ccnc(NC4CCOCC4)c23)c1] | 0.0004 |
| 24 | N-isopropyl-3-(6- (trifluoromethyl)pyrimidin-4- yl)-1H-pyrazolo[4,3- c]pyridin-4-amine | 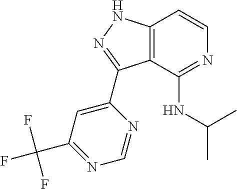 [SMILES: CC(C)Nc1nccc2[nH]nc(-c3cc(C(F)(F)F)ncn3)c12] | 0.0097 |
| 25 | 2-(4-(ethylamino)-1H- pyrazolo[4,3-c]pyridin-3- yl)isonicotinonitrile | 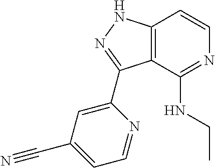 [SMILES: CCNc1nccc2[nH]nc(-c3cc(C#N)ccn3)c12] | 0.0142 |
| 26 | 2-(4-((tetrahydro-2H-pyran- 4-yl)amino)-1H- pyrazolo[4,3-c]pyridin-3- yl)isonicotinonitrile | 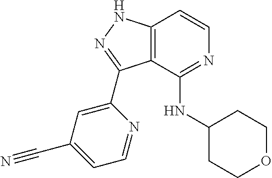 [SMILES: N#Cc1ccnc(-c2n[nH]c3ccnc(NC4CCOCC4)c23)c1] | 0.0038 |
| 27 | N-ethyl-3-(6- (trifluoromethyl)pyrimidin-4- yl)-1H-pyrazolo[4,3- c]pyridin-4-amine | 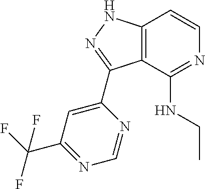 [SMILES: CCNc1nccc2[nH]nc(-c3cc(C(F)(F)F)ncn3)c12] | 0.0318 |
| 28 | 3-(6-methylpyridin-2-yl)-N- (tetrahydro-2H-pyran-4-yl)- 1H-pyrazolo[4,3-c]pyridin-4- amine | 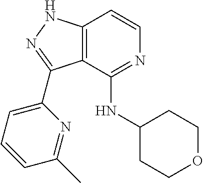 [SMILES: Cc1cccc(-c2n[nH]c3ccnc(NC4CCOCC4)c23)n1] | 0.0018 |
| 29 | 3-(4-chloropyridin-2-yl)-N- (tetrahydro-2H-pyran-4-yl)- 1H-pyrazolo[4,3-c]pyridin-4- amine | 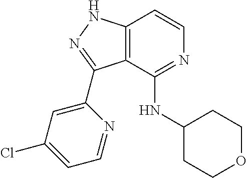 [SMILES: Clc1ccnc(-c2n[nH]c3ccnc(NC4CCOCC4)c23)c1] | 0.0007 |
| 30 | 3-(4-chloropyridin-2-yl)-N- isopropyl-1H-pyrazolo[4,3- c]pyridin-4-amine | 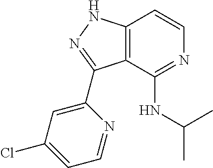 [SMILES: CC(C)Nc1nccc2[nH]nc(-c3cc(Cl)ccn3)c12] | 0.0025 |
| 31 | 2-(4-(isopropylamino)-1H- pyrazolo[4,3-c]pyridin-3- yl)isonicotinonitrile | 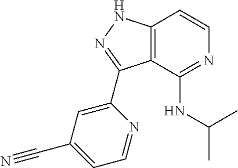 [SMILES: CC(C)Nc1nccc2[nH]nc(-c3cc(C#N)ccn3)c12] | 0.0023 |
| 32 | N-isopropyl-3-(4- (trifluoromethyl)-1H- pyrazol-1-yl)-1H- pyrazolo[4,3-c]pyridin-4- amine | 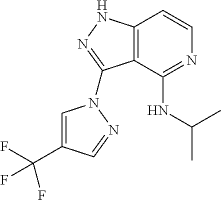 [SMILES: CC(C)Nc1nccc2[nH]nc(-n3cc(C(F)(F)F)cn3)c12] | 0.0091 |
| 33 | 3-(2-methylpyrimidin-4-yl)- N-(tetrahydro-2H-pyran-4- yl)-1H-pyrazolo[4,3- c]pyridin-4-amine | 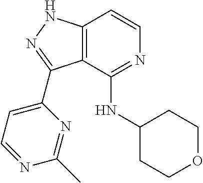 [SMILES: Cc1nccc(-c2n[nH]c3ccnc(NC4CCOCC4)c23)n1] | 0.0010 |
| 34 | N-(4,4-difluorocyclohexyl)- 3-(pyridin-2-yl)-1H- pyrazolo[4,3-c]pyridin-4- amine | 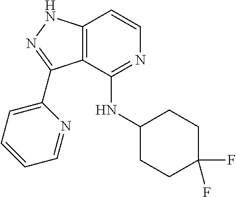 [SMILES: FC1(F)CCC(Nc2nccc3[nH]nc(-c4ccccn4)c23)CC1] | 0.0064 |
| 35 | N-isopropyl-3-(4-(1-methyl- 1H-pyrazol-4-yl)pyridin-2- yl)-1H-pyrazolo[4,3- c]pyridin-4-amine | 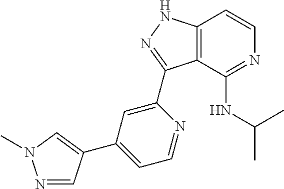 [SMILES: CC(C)Nc1nccc2[nH]nc(-c3cc(-c4cnn(C)c4)ccn3)c12] | 0.0008 |
| 36 | N-isopropyl-3-(4-(1-methyl- 1H-pyrazol-5-yl)pyridin-2- yl)-1H-pyrazolo[4,3- c]pyridin-4-amine | 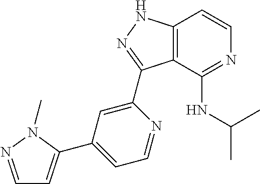 [SMILES: CC(C)Nc1nccc2[nH]nc(-c3cc(-c4ccnn4C)ccn3)c12] | 0.0037 |
| 37 | 3-(2-fluoro-[3,4′-bipyridin]- 2′-yl)-N-isopropyl-1H- pyrazolo[4,3-c]pyridin-4- amine | 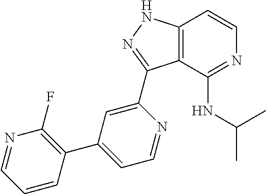 [SMILES: CC(C)Nc1nccc2[nH]nc(-c3cc(-c4cccnc4F)ccn3)c12] | 0.0031 |
| 38 | N-(4,4-difluorocyclohexyl)- 3-(pyrimidin-4-yl)-1H- pyrazolo[4,3-c]pyridin-4- amine | 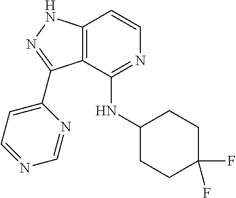 [SMILES: FC1(F)CCC(Nc2nccc3[nH]nc(-c4ccncn4)c23)CC1] | 0.0164 |
| 39 | N-isopropyl-3-(4- (tetrahydro-2H-pyran-4- yl)pyridin-2-yl)-1H- pyrazolo[4,3-c]pyridin-4- amine | 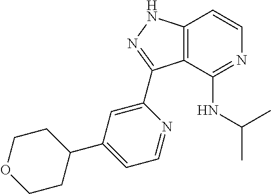 [SMILES: CC(C)Nc1nccc2[nH]nc(-c3cc(C4CCOCC4)ccn3)c12] | 0.0039 |
| 40 | N-isopropyl-3-(4-(1- methylpiperidin-4-yl)pyridin- 2-yl)-1H-pyrazolo[4,3- c]pyridin-4-amine | 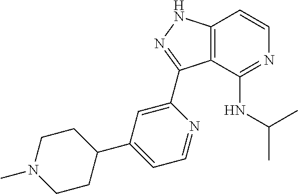 [SMILES: CC(C)Nc1nccc2[nH]nc(-c3cc(C4CCN(C)CC4)ccn3)c12] | 0.0084 |
| 41 | 3-(5-cyclopropyl-4-methyl- 4H-1,2,4-triazol-3-yl)-N- isopropyl-1H-pyrazolo[4,3- c]pyridin-4-amine | 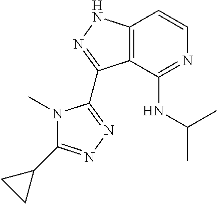 [SMILES: CC(C)Nc1nccc2[nH]nc(-c3nnc(C4CC4)n3C)c12] | 0.151 |
| 42 | 3-(5-cyclopropyl-1-methyl- 1H-1,2,4-triazol-3-yl)-N- isopropyl-1H-pyrazolo[4,3- c]pyridin-4-amine | 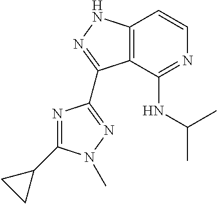 [SMILES: CC(C)Nc1nccc2[nH]nc(-c3nc(C4CC4)n(C)n3)c12] | 0.213 |
| 43 | 3-(pyridin-2-yl)-N-(2,2,2- trifluoroethyl)-1H- pyrazolo[4,3-c]pyridin-4- amine | 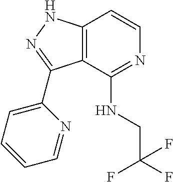 [SMILES: FC(F)(F)CNc1nccc2[nH]nc(-c3ccccn3)c12] | 0.0088 |
| 44 | 3-(4-(1,5-dimethyl-1H- pyrazol-4-yl)pyridin-2-yl)-N- isopropyl-1H-pyrazolo[4,3- c]pyridin-4-amine | 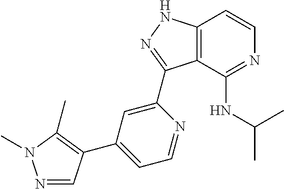 [SMILES: Cc1c(-c2ccnc(-c3n[nH]c4ccnc(NC(C)C)c34)c2)cnn1C] | 0.0011 |
| 45 | 3-(4-(1,3-dimethyl-1H- pyrazol-4-yl)pyridin-2-yl)-N- isopropyl-1H-pyrazolo[4,3- c]pyridin-4-amine | 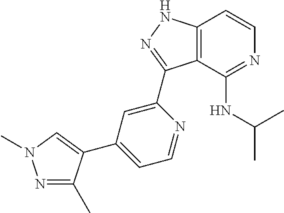 [SMILES: Cc1nn(C)cc1-c1ccnc(-c2n[nH]c3ccnc(NC(C)C)c23)c1] | 0.0013 |
| 46 | N-isopropyl-3-(4-(1-methyl- 1H-pyrazol-3-yl)pyridin-2- yl)-1H-pyrazolo[4,3- c]pyridin-4-amine | 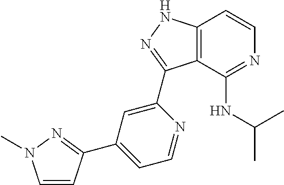 [SMILES: CC(C)Nc1nccc2[nH]nc(-c3cc(-c4ccn(C)n4)ccn3)c12] | 0.0010 |
| 47 | N-isopropyl-3-(6- (tetrahydro-2H-pyran-4- yl)pyrimidin-4-yl)-1H- pyrazolo[4,3-c]pyridin-4- amine | 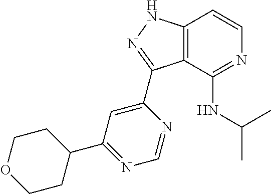 [SMILES: CC(C)Nc1nccc2[nH]nc(-c3cc(C4CCOCC4)ncn3)c12] | 0.0038 |
| 48 | N-isopropyl-3-(6- (tetrahydrofuran-3- yl)pyrimidin-4-yl)-1H- pyrazolo[4,3-c]pyridin-4- amine | 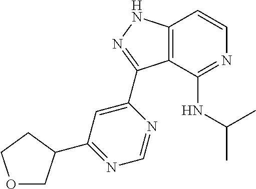 [SMILES: CC(C)Nc1nccc2[nH]nc(-c3cc(C4CCOC4)ncn3)c12] | 0.012 |
| 49 | N-isopropyl-3-(4- (tetrahydrofuran-3- yl)pyridin-2-yl)-1H- pyrazolo[4,3-c]pyridin-4- amine | 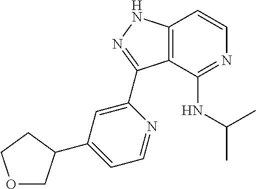 [SMILES: CC(C)Nc1nccc2[nH]nc(-c3cc(C4CCOC4)ccn3)c12] | 0.0046 |
| 50 | 3-(4-ethoxypyridin-2-yl)-N- (tetrahydro-2H-pyran-4-yl)- 1H-pyrazolo[4,3-c]pyridin-4- amine | 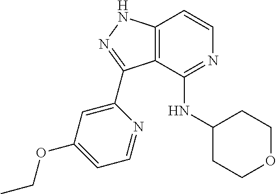 [SMILES: CCOc1ccnc(-c2n[nH]c3ccnc(NC4CCOCC4)c23)c1] | 0.0022 |
| 51 | 6-methyl-N-(tetrahydro-2H- pyran-4-yl)-3-(4- (trifluoromethyl)pyridin-2- yl)-1H-pyrazolo[4,3- c]pyridin-4-amine | 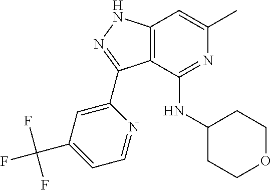 [SMILES: Cc1cc2[nH]nc(-c3cc(C(F)(F)F)ccn3)c2c(NC2CCOCC2)n1] | 0.0399 |
| 52 | N,N-dimethyl-2-(4- ((tetrahydro-2H-pyran-4- yl)amino)-1H-pyrazolo[4,3- c]pyridin-3- yl)isonicotinamide | 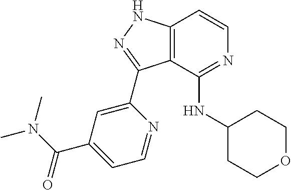 [SMILES: CN(C)C(=O)c1ccnc(-c2n[nH]c3ccnc(NC4CCOCC4)c23)c1] | 0.0372 |
| 53 | N-isopropyl-3-(4- (tetrahydrofuran-2- yl)pyridin-2-yl)-1H- pyrazolo[4,3-c]pyridin-4- amine | 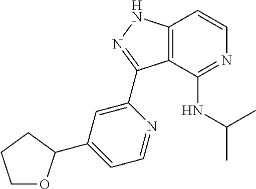 [SMILES: CC(C)Nc1nccc2[nH]nc(-c3cc(C4CCCO4)ccn3)c12] | 0.0044 |
| 54 | N-isopropyl-3-(6- (tetrahydrofuran-2- yl)pyrimidin-4-yl)-1H- pyrazolo[4,3-c]pyridin-4- amine | 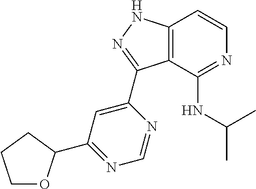 [SMILES: CC(C)Nc1nccc2[nH]nc(-c3cc(C4CCCO4)ncn3)c12] | 0.0287 |
| 55 | 2-(4-(isopropylamino)-1H- pyrazolo[4,3-c]pyridin-3-yl)- N,N- dimethylisonicotinamide | 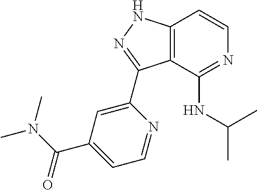 [SMILES: CC(C)Nc1nccc2[nH]nc(-c3cc(C(=O)N(C)C)ccn3)c12] | 0.0469 |
| 56 | (2-(4-(isopropylamino)-1H- pyrazolo[4,3-c]pyridin-3- yl)pyridin-4- yl)(morpholino)methanone | 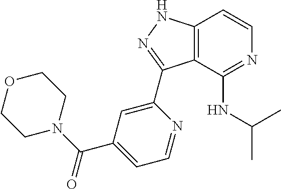 [SMILES: CC(C)Nc1nccc2[nH]nc(-c3cc(C(=O)N4CCOCC4)ccn3)c12] | 0.0803 |
| 57 | 6-methyl-N-(tetrahydro-2H- pyran-4-yl)-3-(6- (trifluoromethyl)pyrimidin-4- yl)-1H-pyrazolo[4,3- c]pyridin-4-amine | 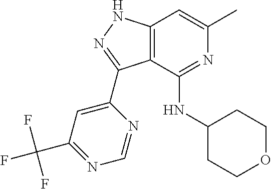 [SMILES: Cc1cc2[nH]nc(-c3cc(C(F)(F)F)ncn3)c2c(NC2CCOCC2)n1] | 0.0663 |
| 58 | 3-(6-ethoxypyrimidin-4-yl)- 6-methyl-N-(tetrahydro-2H- pyran-4-yl)-1H-pyrazolo[4,3- c]pyridin-4-amine | 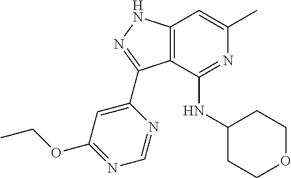 [SMILES: CCOc1cc(-c2n[nH]c3cc(C)nc(NC4CCOCC4)c23)ncn1] | 0.0391 |
| 59 | 3-(4-((3R,4R)-3,4- difluoropyrrolidin-1- yl)pyridin-2-yl)-N-isopropyl- 1H-pyrazolo[4,3-c]pyridin-4- amine | 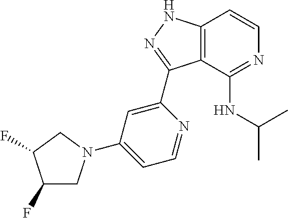 [SMILES: CC(C)Nc1nccc2[nH]nc(-c3cc(N4C[C@@H](F)[C@H](F)C4)ccn3)c12] | 0.0039 |
| 60 | N,N-dimethyl-2-(6-methyl- 4-((tetrahydro-2H-pyran-4- yl)amino)-1H-pyrazolo[4,3- c]pyridin-3- yl)isonicotinamide | 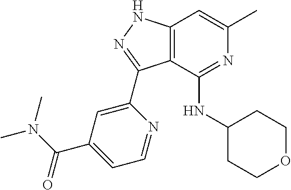 [SMILES: Cc1cc2[nH]nc(-c3cc(C(=O)N(C)C)ccn3)c2c(NC2CCOCC2)n1] | 0.644 |
| 61 | 3-(4-(2,2,6,6-tetrafluoro- morpholino)pyridin- 2-yl)-N-(tetrahydro-2H- pyran-4-yl)-1H-pyrazolo[4,3- c]pyridin-4-amine | 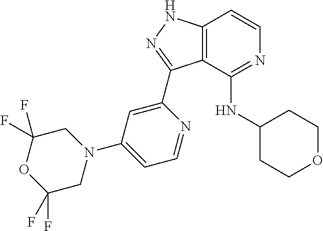 [SMILES: FC1(F)CN(c2ccnc(-c3n[nH]c4ccnc(NC5CCOCC5)c34)c2)CC(F)(F)O1] | 0.0014 |
| 62 | 6-(4-(isopropylamino)-1H- pyrazolo[4,3-c]pyridin-3-yl)- N,N-dimethylpyrimidine-4- carboxamide | 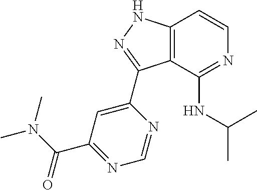 [SMILES: CC(C)Nc1nccc2[nH]nc(-c3cc(C(=O)N(C)C)ncn3)c12] | 0.193 |
| 63 | azetidin-1-yl(2-(4- ((tetrahydro-2H-pyran-4- yl)amino)-1H-pyrazolo[4,3- c]pyridin-3-yl)pyridin-4- yl)methanone | 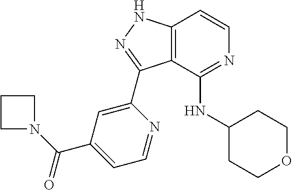 [SMILES: O=C(c1ccnc(-c2n[nH]c3ccnc(NC4CCOCC4)c23)c1)N1CCC1] | 0.0147 |
| 64 | N,N,2-trimethyl-6-(4- ((tetrahydro-2H-pyran-4- yl)amino)-1H-pyrazolo[4,3- c]pyridin-3- yl)isonicotinamide | 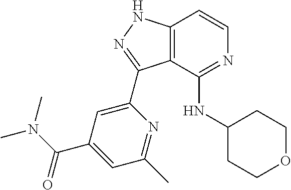 [SMILES: Cc1cc(C(=O)N(C)C)cc(-c2n[nH]c3ccnc(NC4CCOCC4)c23)n1] | 0.0166 |
| 65 | N-(cyclopropylmethyl)-3-(6- (trifluoromethyl)pyrimidin-4- yl)-1H-pyrazolo[4,3- c]pyridin-4-amine | 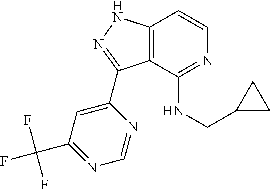 [SMILES: FC(F)(F)c1cc(-c2n[nH]c3ccnc(NCC4CC4)c23)ncn1] | 0.0202 |
| 66 | N-(4,4-difluorocyclohexyl)- 3-(6-(trifluoro- methyl)pyrimidin-4- yl)-1H-pyrazolo[4,3- c]pyridin-4-amine | 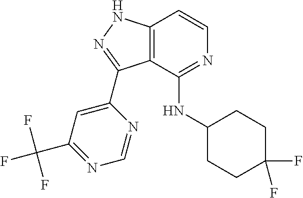 [SMILES: FC1(F)CCC(Nc2nccc3[nH]nc(-c4cc(C(F)(F)F)ncn4)c23)CC1] | 0.0229 |
| 67 | N-(4,4-difluorocyclohexyl)- 3-(2-methyl-6- (trifluoromethyl)pyrimidin-4- yl)-1H-pyrazolo[4,3- c]pyridin-4-amine | 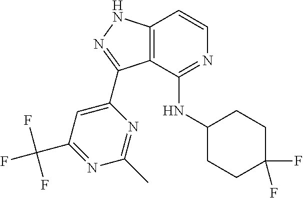 [SMILES: Cc1nc(-c2n[nH]c3ccnc(NC4CCC(F)(F)CC4)c23)cc(C(F)(F)F)n1] | 0.0203 |
| 68 | N-(2,6-dimethyltetrahydro- 2H-pyran-4-yl)-3-(6- (trifluoromethyl)pyrimidin-4- yl)-1H-pyrazolo[4,3- c]pyridin-4-amine | 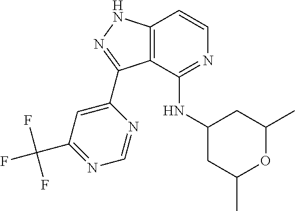 [SMILES: CC1CC(Nc2nccc3[nH]nc(-c4cc(C(F)(F)F)ncn4)c23)CC(C)O1] | 0.0233 |
| 69 | 3-(4- (methylsulfonyl)pyridin-2- yl)-N-(tetrahydro-2H-pyran- 4-yl)-1H-pyrazolo[4,3- c]pyridin-4-amine | 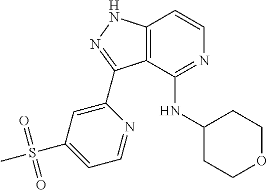 [SMILES: CS(=O)(=O)c1ccnc(-c2n[nH]c3ccnc(NC4CCOCC4)c23)c1] | 0.0068 |
| 70 | 3-(6-methyl-4- (trifluoromethyl)pyridin-2- yl)-N-(tetrahydro-2H-pyran- 4-yl)-1H-pyrazolo[4,3- c]pyridin-4-amine | 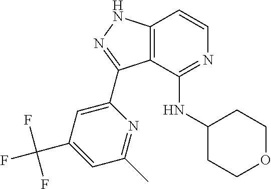 [SMILES: Cc1cc(C(F)(F)F)cc(-c2n[nH]c3ccnc(NC4CCOCC4)c23)n1] | 0.0007 |
| 71 | 3-(2-methyl-6- (trifluoromethyl)pyrimidin-4- yl)-N-(tetrahydro-2H-pyran- 4-yl)-1H-pyrazolo[4,3- c]pyridin-4-amine | 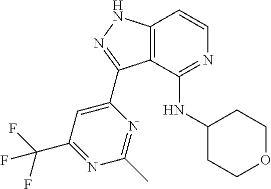 [SMILES: Cc1nc(-c2n[nH]c3ccnc(NC4CCOCC4)c23)cc(C(F)(F)F)n1] | 0.0067 |
| 72 | N-isopropyl-3-(4- (methylsulfonyl)pyridin-2- yl)-1H-pyrazolo[4,3- c]pyridin-4-amine | 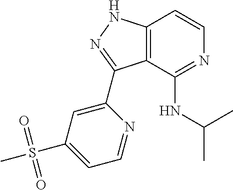 [SMILES: CC(C)Nc1nccc2[nH]nc(-c3cc(S(C)(=O)=O)ccn3)c12] | 0.0060 |
| 73 | N-(dicyclopropylmethyl)- 3-(6-(trifluoro- methyl)pyrimidin-4- yl)-1H-pyrazolo[4,3- c]pyridin-4-amine | 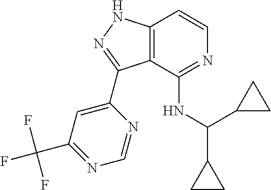 [SMILES: FC(F)(F)c1cc(-c2n[nH]c3ccnc(NC(C4CC4)C4CC4)c23)ncn1] | 0.0025 |
| 74 | N-isopropyl-3-(4- (isopropylsulfonyl)pyridin-2- yl)-1H-pyrazolo[4,3- c]pyridin-4-amine | 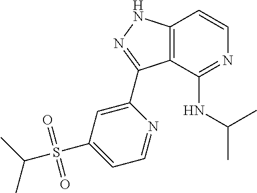 [SMILES: CC(C)Nc1nccc2[nH]nc(-c3cc(S(=O)(=O)C(C)C)ccn3)c12] | 0.0258 |
| 75 | 3-(4- (isopropylsulfonyl)pyridin-2- yl)-N-(tetrahydro-2H-pyran- 4-yl)-1H-pyrazolo[4,3- c]pyridin-4-amine | 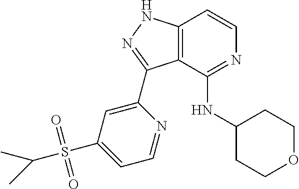 [SMILES: CC(C)S(=O)(=O)c1ccnc(-c2n[nH]c3ccnc(NC4CCOCC4)c23)c1] | 0.0232 |
| 76 | N-isopropyl-3-(6-methyl-4- (methylsulfonyl)pyridin-2- yl)-1H-pyrazolo[4,3- c]pyridin-4-amine | 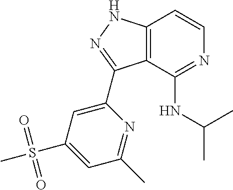 [SMILES: Cc1cc(S(C)(=O)=O)cc(-c2n[nH]c3ccnc(NC(C)C)c23)n1] | 0.0045 |
| 77 | N-(8-oxabicyclo[3.2.1]octan- 3-yl)-3-(6- (trifluoromethyl)pyrimidin-4- yl)-1H-pyrazolo[4,3- c]pyridin-4-amine | 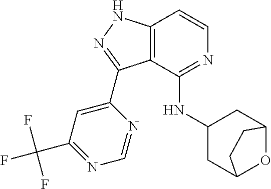 [SMILES: FC(F)(F)c1cc(-c2n[nH]c3ccnc(NC4CC5CCC(C4)O5)c23)ncn1] | 0.0226 |

TABLE 2
Structures and K_{I}(μM) values for selected compounds according to the prior art

| Compound | Structure | Ki (μM) |
|---|---|---|
| Example 154 WO 2010106333 | 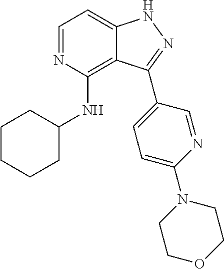 [SMILES: c1cc2[nH]nc(-c3ccc(N4CCOCC4)nc3)c2c(NC2CCCCC2)n1] | ** |
| Example 160 WO 2010106333 | 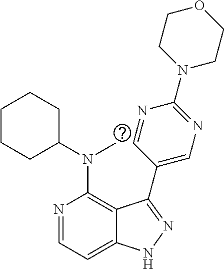 [SMILES: CN(c1nccc2[nH]nc(-c3cnc(N4CCOCC4)nc3)c12)C1CCCCC1] | ** |
| Example 162 WO 2010106333 | 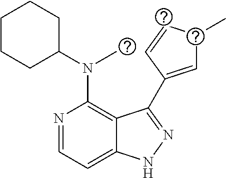 [SMILES: CC1C=CC(c2n[nH]c3ccnc(N(C)C4CCCCC4)c23)=C1] | ** |
| Example 164 WO 2010106333 | 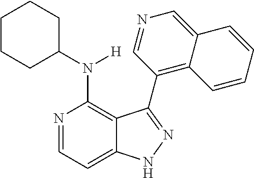 [SMILES: c1ccc2c(-c3n[nH]c4ccnc(NC5CCCCC5)c34)cncc2c1] | * |
| Example 233 WO 2010106333 | 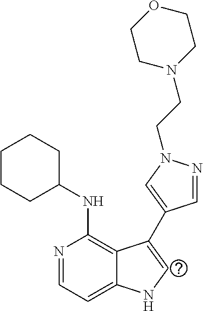 [SMILES: c1cc2[nH]cc(-c3cnn(CCN4CCOCC4)c3)c2c(NC2CCCCC2)n1] | ** |
| Example 234 WO 2010106333 | 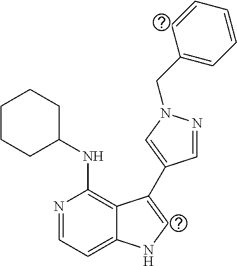 [SMILES: c1ccc(Cn2cc(-c3c[nH]c4ccnc(NC5CCCCC5)c34)cn2)cc1] | ** |
| Example 235 WO 2010106333 | 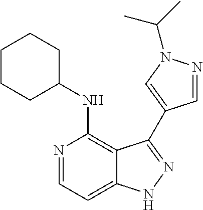 [SMILES: CC(C)n1cc(-c2n[nH]c3ccnc(NC4CCCCC4)c23)cn1] | *** |
| Example 16 WO 2011141756 | 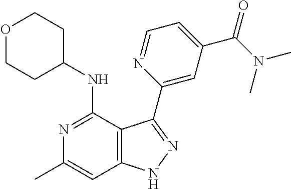 [SMILES: Cc1cc2[nH]nc(-c3cc(C(=O)N(C)C)ccn3)c2c(NC2CCOCC2)n1] | ** |
| Example 17 WO 2011141756 | 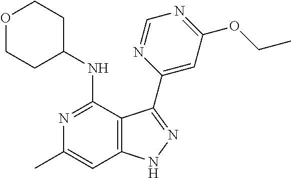 [SMILES: CCOc1cc(-c2n[nH]c3cc(C)nc(NC4CCOCC4)c23)ncn1] | *** |
| Example 18 WO 2011141756 | 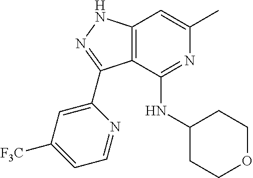 [SMILES: Cc1cc2[nH]nc(-c3cc(C(F)(F)F)ccn3)c2c(NC2CCOCC2)n1] | *** |
| Example 19 WO 2011141756 | 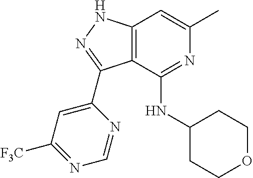 [SMILES: Cc1cc2[nH]nc(-c3cc(C(F)(F)F)ncn3)c2c(NC2CCOCC2)n1] | *** |

*** = LRRK2 IC50 < 100 nM
** = LRRK2 IC50 between 100 nM and 1 μM
* = LRRK2 IC50 between 1 μM and 10 μM

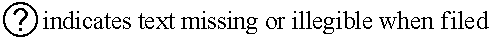

1. A compound of formula (IA) or (IB), or a pharmaceutically acceptable salt thereof, 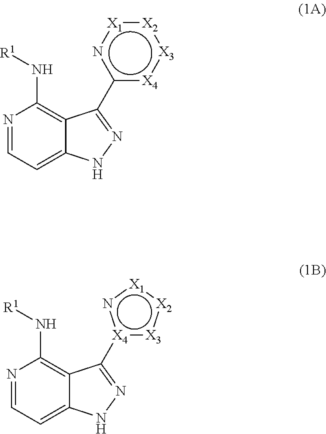
wherein:
R¹ is a group selected from alkyl, monocyclic heterocycloalkyl, bicyclic heterocycloalkyl and cycloalkyl, each of which is optionally substituted by one or more groups selected from alkyl, halo and cycloalkyl;
where for formula (1A):
one, two or three of X₁, X₂, X₃ and X₄ are N, and the remainder are each independently CR²; or
X₁, X₂, X₃ and X₄ are each independently CR²; or
where for formula (1B):
X⁴ is C or N; and
one or two of X₁, X₂ and X₃ are independently selected from N and NR⁸, and the remainder are each independently CR²;
such that X₁, X₂, X₃, X₄ and N form a heteroaryl group;
each R² is independently selected from H, alkyl, CN, halo, heteroaryl, heterocycloalkyl, cycloalkyl, OR⁴, CONR⁵R⁶ and SO₂R⁷, wherein said alkyl, heteroaryl, heterocycloalkyl and cycloalkyl groups are each optionally further substituted by one or more groups selected from alkyl, halo and OR⁹;
each R⁸ is independently selected from H and alkyl, wherein said alkyl group is optionally further substituted by one or more groups selected from CN, halo, heteroaryl, heterocycloalkyl, cycloalkyl, OR⁴, CONR⁵R⁶ and SO₂R⁷;
R⁴, R⁵, R⁶, R⁷ and R⁹ are each independently selected from H and alkyl; or
R⁵ and R⁶ together with the nitrogen to which they are attached are linked to form a cyclic group which optionally further comprises one or more heteroatoms selected from O, N and S;
with the proviso that when the compound is of formula (IB), the compound is other than 3-(5-isopropyl-4H-1,2,4-triazol-3-yl)-N-(tetrahydro-2H-pyran-4-yl)-1H-pyrazolo[4,3-c]pyridin-4-amine or 3-(5-cyclopropyl-4H-1,2,4-triazol-3-yl)-N-(tetrahydro-2H-pyran-4-yl)-1H-pyrazolo[4,3-c]pyridin-4-amine.

2. A compound according to claim 1 of formula (IA) or (IB), or a pharmaceutically acceptable salt thereof, 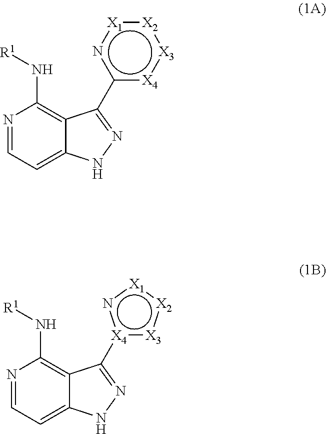
wherein:
R¹ is a group selected from alkyl, monocyclic heterocycloalkyl, bicyclic heterocycloalkyl and cycloalkyl, each of which is optionally substituted by one or more groups selected from alkyl, halo and cycloalkyl;
where for formula (1A):
one, two or three of X₁, X₂, X₃ and X₄ are N, and the remainder are each independently CR²; or
where for formula (1B):
X⁴ is C or N; and
one or two of X₁, X₂ and X₃ are independently selected from N and NR⁸, and the remainder are each independently CR²;
such that X₁, X₂, X₃, X₄ and N form a heteroaryl group;
each R² is independently selected from H, alkyl, CN, halo, heteroaryl, heterocycloalkyl, cycloalkyl, OR⁴, CONR⁵R⁶ and SO₂R⁷, wherein said alkyl, heteroaryl, heterocycloalkyl and cycloalkyl groups are each optionally further substituted by one or more groups selected from alkyl, halo and OR⁹;
each R⁸ is independently selected from H and alkyl, wherein said alkyl group is optionally further substituted by one or more groups selected from CN, halo, heteroaryl, heterocycloalkyl, cycloalkyl, OR⁴, CONR⁵R⁶ and SO₂R⁷;
R⁴, R⁵, R⁶, R⁷ and R⁹ are each independently selected from H and alkyl; or
R⁵ and R⁶ together with the nitrogen to which they are attached are linked to form a cyclic group which optionally further comprises one or more heteroatoms selected from O, N and S;
with the proviso that when the compound is of formula (IB), the compound is other than 3-(5-isopropyl-4H-1,2,4-triazol-3-yl)-N-(tetrahydro-2H-pyran-4-yl)-1H-pyrazolo[4,3-c]pyridin-4-amine or 3-(5-cyclopropyl-4H-1,2,4-triazol-3-yl)-N-(tetrahydro-2H-pyran-4-yl)-1H-pyrazolo[4,3-c]pyridin-4-amine.

3. A compound according to claim 1 which is of formula (1A), wherein
X₄ is N and X₁, X₂ and X₃ are each independently CR².

4. A compound according to claim 1 which is of formula (1A), wherein
X₂ is N and X₁, X₃ and X₄ are each independently CR².

5. A compound according to claim 1 which is of formula (1A), wherein
X₁, X₂, X₃ and X₄ are each independently CR².

6. A compound according to claim 1 which is of formula (1A), wherein:
(i) X₄ is N, X₁ and X₂ are CH, and X₃ is CR²;
(ii) X₂ is N, X₁ and X₄ are CH, and X₃ is CR²; or
(iii) X₁, X₂ and X₄ are CH and X₃ is CR².

7. A compound according to claim 1 which is of formula (1B), wherein
X⁴ is C, X₁ is NR⁸, and X₂ and X₃ are each independently CR².

8. A compound according to claim 1 which is of formula (1B), wherein
X⁴ is C, X₃ is CR², one of X₁ and X₂ is N and the other is NR⁸.

9. A compound according to claim 1 which is of formula (1B), wherein
X⁴ is C, X₂ is NR⁸, and X₁ and X₃ are each independently CR².

10. A compound according to claim 1 which is of formula (1B), wherein
X⁴ is N, and X₁, X₂ and X₃ are each independently CR².

11. A compound according to claim 1 which is of formula (1B), wherein
X⁴ is C, X₂ is CR², one of X₁ and X₃ is N and the other is NR⁸.

12. A compound according to claim 1 which is of formula (1B), wherein:
(i) X⁴ is C, X₁ is NR⁸, and X₃ is CH and X₂ is CR²;
(ii) X⁴ is C, X₃ is CH, X₁ is N and X₂ is NR⁸;
(iii) X⁴ is C, X₂ is NR⁸, and X₁ and X₃ are each independently CH;
(iv) X⁴ is N, X₁, and X₃ are CH and X₂ is CR²; or
(v) X⁴ is C, X₂ is CR², one of X₁ and X₃ is N and the other is NR⁸.

13. A compound according to claim 1 wherein
R¹ is selected from:
(i) an isopropyl or ethyl group, each of which is optionally substituted by a group selected from halo and cyclopropyl;
(ii) a tetrahydropyranyl group or 8-oxabicyclo[3.2.1]octan-3-yl group, each of which is optionally substituted by one or more alkyl groups;
(ii) a cyclohexyl group optionally substituted by one or more halo groups.

14. A compound according to claim 1 wherein
R² is selected from:
(i) an alkyl group optionally substituted by one or more halo or OR⁴ groups, where R⁴ is alkyl;
(ii) a heteroaryl group optionally substituted by one or more groups selected from alkyl and halo;
(iii) a heterocycloalkyl group optionally substituted by one or more groups selected from alkyl and halo;
(iv) a cycloalkyl group optionally substituted by one or more groups selected from alkyl and halo;
(v) CN;
(vi) halo;
(vii) SO₂R⁷ where R⁷ is alkyl;
(viii) OR⁴, where R⁴ is alkyl;
(ix) CONR⁵R⁶ where R⁵ and R⁶ are both alkyl, or R⁵ and R⁶ together with the nitrogen to which they are attached are linked to form a 4, 5 or 6-membered heterocycloalkyl group.

15. A compound according to claim 13 wherein R² is selected from:
(i) a methyl, ethyl or isopropyl group optionally substituted by one or more halo or OR⁴ groups, where R⁴ is alkyl;
(ii) a heteroaryl group selected from a pyrazolyl group and a pyridinyl group, each of which is optionally substituted by one or more alkyl or halo groups;
(iii) a heterocycloalkyl group selected from morpholinyl, tetrahydropyranyl, piperidinyl and tetrahydrofuranyl, each of which is optionally substituted by one or more alkyl or halo groups;
(iv) a cyclopropyl group optionally substituted by one or more alkyl or halo groups;
(v) CN;
(vi) halo;
(vii) SO₂R⁷ where R⁷ is methyl or isopropyl;
(viii) OR⁴, where R⁴ is ethyl;
(ix) CONR⁵R⁶ where R⁵ and R⁶ are both methyl, or R⁵ and R⁶ together with the nitrogen to which they are attached are linked to form a morpholinyl group, or R⁵ and R⁶ together with the nitrogen to which they are attached are linked to form an azetidinyl group.

16. A compound according to claim 1 which is selected from the following:

| | Name | Structure |
|---|---|---|
| 1 | 3-(5-ethyl-1-methyl-1H-pyrazol-3-yl)-N- (tetrahydro-2H-pyran-4-yl)-1H-pyrazolo[4,3- c]pyridin-4-amine | 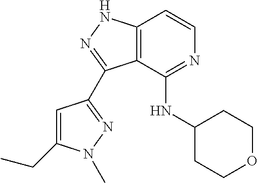 [SMILES: CCc1cc(-c2n[nH]c3ccnc(NC4CCOCC4)c23)nn1C] |
| 2 | N-isopropyl-3-(4-morpholinopyrimidin-2-yl)- 1H-pyrazolo[4,3-c]pyridin-4-amine | 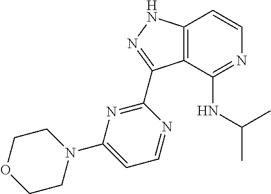 [SMILES: CC(C)Nc1nccc2[nH]nc(-c3nccc(N4CCOCC4)n3)c12] |
| 3 | N-isopropyl-3-(4-morpholinopyridin-2-yl)-1H- pyrazolo[4,3-c]pyridin-4-amine | 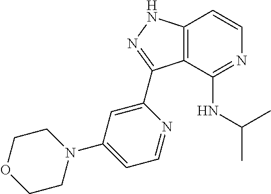 [SMILES: CC(C)Nc1nccc2[nH]nc(-c3cc(N4CCOCC4)ccn3)c12] |
| 4 | N-isopropyl-3-(6-morpholinopyrimidin-4-yl)- 1H-pyrazolo[4,3-c]pyridin-4-amine | 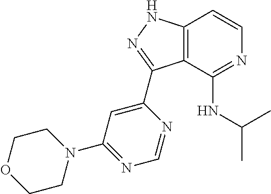 [SMILES: CC(C)Nc1nccc2[nH]nc(-c3cc(N4CCOCC4)ncn3)c12] |
| 5 | 3-(1-isopropyl-1H-1,2,3-triazol-4-yl)-N- (tetrahydro-2H-pyran-4-yl)-1H-pyrazolo[4,3- c]pyridin-4-amine | 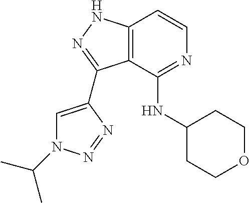 [SMILES: CC(C)n1cc(-c2n[nH]c3ccnc(NC4CCOCC4)c23)nn1] |
| 6 | 3-(1-isopropyl-1H-imidazol-4-yl)-N-(tetrahydro- 2H-pyran-4-yl)-1H-pyrazolo[4,3-c]pyridin-4- amine | 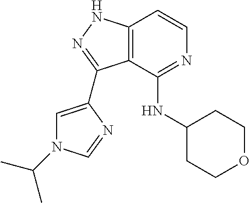 [SMILES: CC(C)n1cnc(-c2n[nH]c3ccnc(NC4CCOCC4)c23)c1] |
| 7 | N-isopropyl-3-(1-isopropyl-1H-1,2,3-triazol-4- yl)-1H-pyrazolo[4,3-c]pyridin-4-amine | 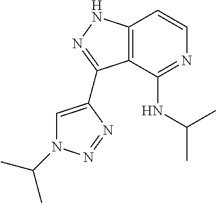 [SMILES: CC(C)Nc1nccc2[nH]nc(-c3cn(C(C)C)nn3)c12] |
| 8 | N-isopropyl-3-(1-methyl-1H-1,2,3-triazol-4-yl)- 1H-pyrazolo[4,3-c]pyridin-4-amine | 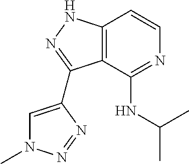 [SMILES: CC(C)Nc1nccc2[nH]nc(-c3cn(C)nn3)c12] |
| 9 | N-ethyl-3-(1-isopropyl-1H-1,2,3-triazol-4-yl)- 1H-pyrazolo[4,3-c]pyridin-4-amine | 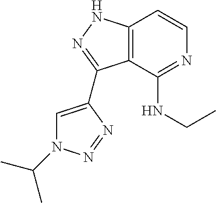 [SMILES: CCNc1nccc2[nH]nc(-c3cn(C(C)C)nn3)c12] |
| 10 | N-ethyl-3-(1-methyl-1H-1,2,3-triazol-4-yl)-1H- pyrazolo[4,3-c]pyridin-4-amine | 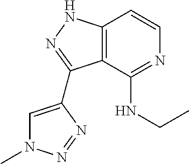 [SMILES: CCNc1nccc2[nH]nc(-c3cn(C)nn3)c12] |
| 11 | 3-(5-ethyl-1-methyl-1H-pyrazol-3-yl)-N- isopropyl-1H-pyrazolo[4,3-c]pyridin-4-amine | 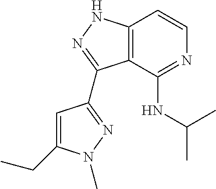 [SMILES: CCc1cc(-c2n[nH]c3ccnc(NC(C)C)c23)nn1C] |
| 12 | N-ethyl-3-(1-methyl-1H-imidazol-4-yl)-1H- pyrazolo[4,3-c]pyridin-4-amine | 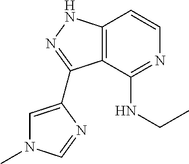 [SMILES: CCNc1nccc2[nH]nc(-c3cn(C)cn3)c12] |
| 13 | N-isopropyl-3-(pyridin-2-yl)-1H-pyrazolo[4,3- c]pyridin-4-amine | 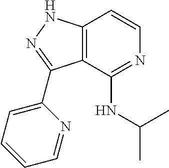 [SMILES: CC(C)Nc1nccc2[nH]nc(-c3ccccn3)c12] |
| 14 | N-ethyl-3-(1-(2-methoxyethyl)-1H-1,2,3-triazol- 4-yl)-1H-pyrazolo[4,3-c]pyridin-4-amine | 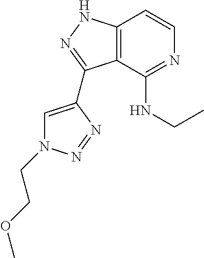 [SMILES: CCNc1nccc2[nH]nc(-c3cn(CCOC)nn3)c12] |
| 15 | N-isopropyl-3-(pyrimidin-4-yl)-1H- pyrazolo[4,3-c]pyridin-4-amine | 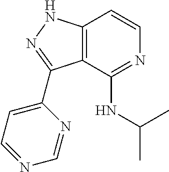 [SMILES: CC(C)Nc1nccc2[nH]nc(-c3ccncn3)c12] |
| 16 | N-ethyl-3-(pyridin-2-yl)-1H-pyrazolo[4,3- c]pyridin-4-amine | 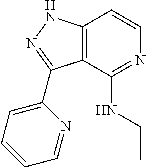 [SMILES: CCNc1nccc2[nH]nc(-c3ccccn3)c12] |
| 17 | 3-(pyridin-2-yl)-N-(tetrahydro-2H-pyran-4-yl)- 1H-pyrazolo[4,3-c]pyridin-4-amine | 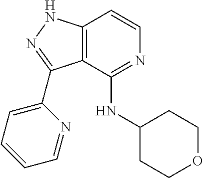 [SMILES: c1ccc(-c2n[nH]c3ccnc(NC4CCOCC4)c23)nc1] |
| 18 | 3-(pyrimidin-4-yl)-N-(tetrahydro-2H-pyran-4- yl)-1H-pyrazolo[4,3-c]pyridin-4-amine | 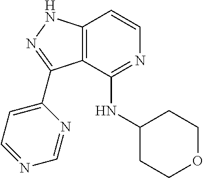 [SMILES: c1cc(-c2n[nH]c3ccnc(NC4CCOCC4)c23)ncn1] |
| 19 | N-ethyl-3-(pyrimidin-4-yl)-1H-pyrazolo[4,3- c]pyridin-4-amine | 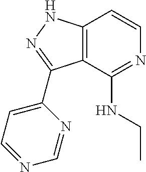 [SMILES: CCNc1nccc2[nH]nc(-c3ccncn3)c12] |
| 20 | N-isopropyl-3-(4-(trifluoromethyl)pyridin-2-yl)- 1H-pyrazolo[4,3-c]pyridin-4-amine | 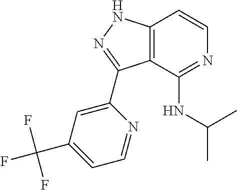 [SMILES: CC(C)Nc1nccc2[nH]nc(-c3cc(C(F)(F)F)ccn3)c12] |
| 21 | N-ethyl-3-(4-(trifluoromethyl)pyridin-2-yl)-1H- pyrazolo[4,3-c]pyridin-4-amine | 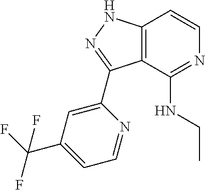 [SMILES: CCNc1nccc2[nH]nc(-c3cc(C(F)(F)F)ccn3)c12] |
| 22 | N-(tetrahydro-2H-pyran-4-yl)-3-(6- (trifluoromethyl)pyrimidin-4-yl)-1H- pyrazolo[4,3-c]pyridin-4-amine | 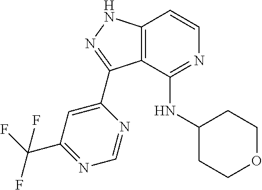 [SMILES: FC(F)(F)c1cc(-c2n[nH]c3ccnc(NC4CCOCC4)c23)ncn1] |
| 23 | N-(tetrahydro-2H-pyran-4-yl)-3-(4- (trifluoromethyl)pyridin-2-yl)-1H-pyrazolo[4,3- c]pyridin-4-amine | 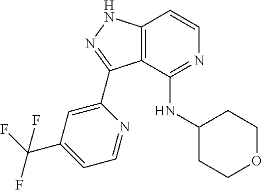 [SMILES: FC(F)(F)c1ccnc(-c2n[nH]c3ccnc(NC4CCOCC4)c23)c1] |
| 24 | N-isopropyl-3-(6-(trifluoromethyl)pyrimidin-4- yl)-1H-pyrazolo[4,3-c]pyridin-4-amine | 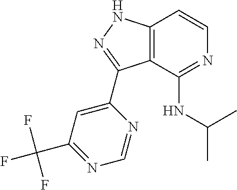 [SMILES: CC(C)Nc1nccc2[nH]nc(-c3cc(C(F)(F)F)ncn3)c12] |
| 25 | 2-(4-(ethylamino)-1H-pyrazolo[4,3-c]pyridin-3- yl)isonicotinonitrile | 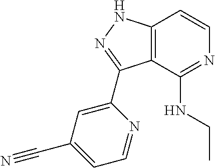 [SMILES: CCNc1nccc2[nH]nc(-c3cc(C#N)ccn3)c12] |
| 26 | 2-(4-((tetrahydro-2H-pyran-4-yl)amino)-1H- pyrazolo[4,3-c]pyridin-3-yl)isonicotinonitrile | 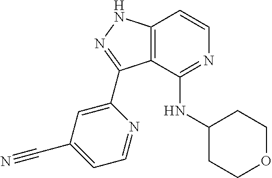 [SMILES: N#Cc1ccnc(-c2n[nH]c3ccnc(NC4CCOCC4)c23)c1] |
| 27 | N-ethyl-3-(6-(trifluoromethyl)pyrimidin-4-yl)- 1H-pyrazolo[4,3-c]pyridin-4-amine | 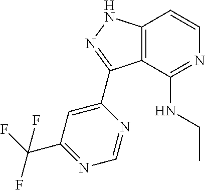 [SMILES: CCNc1nccc2[nH]nc(-c3cc(C(F)(F)F)ncn3)c12] |
| 28 | 3-(6-methylpyridin-2-yl)-N-(tetrahydro-2H- pyran-4-yl)-1H-pyrazolo[4,3-c]pyridin-4-amine | 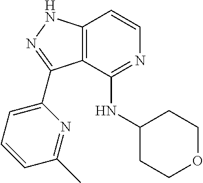 [SMILES: Cc1cccc(-c2n[nH]c3ccnc(NC4CCOCC4)c23)n1] |
| 29 | 3-(4-chloropyridin-2-yl)-N-(tetrahydro-2H- pyran-4-yl)-1H-pyrazolo[4,3-c]pyridin-4-amine | 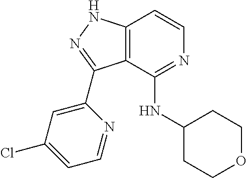 [SMILES: Clc1ccnc(-c2n[nH]c3ccnc(NC4CCOCC4)c23)c1] |
| 30 | 3-(4-chloropyridin-2-yl)-N-isopropyl-1H- pyrazolo[4,3-c]pyridin-4-amine | 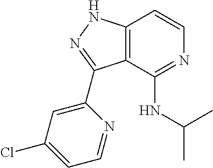 [SMILES: CC(C)Nc1nccc2[nH]nc(-c3cc(Cl)ccn3)c12] |
| 31 | 2-(4-(isopropylamino)-1H-pyrazolo[4,3- c]pyridin-3-yl)isonicotinonitrile | 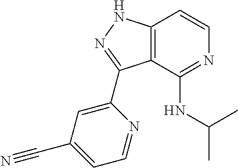 [SMILES: CC(C)Nc1nccc2[nH]nc(-c3cc(C#N)ccn3)c12] |
| 32 | N-isopropyl-3-(4-(trifluoromethyl)-1H-pyrazol- 1-yl)-1H-pyrazolo[4,3-c]pyridin-4-amine | 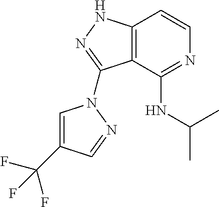 [SMILES: CC(C)Nc1nccc2[nH]nc(-n3cc(C(F)(F)F)cn3)c12] |
| 33 | 3-(2-methylpyrimidin-4-yl)-N-(tetrahydro-2H- pyran-4-yl)-1H-pyrazolo[4,3-c]pyridin-4-amine | 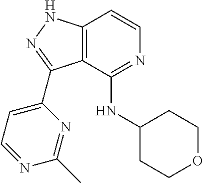 [SMILES: Cc1nccc(-c2n[nH]c3ccnc(NC4CCOCC4)c23)n1] |
| 34 | N-(4,4-difluorocyclohexyl)-3-(pyridin-2-yl)-1H- pyrazolo[4,3-c]pyridin-4-amine | 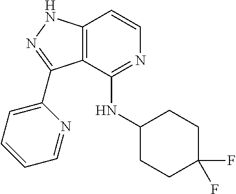 [SMILES: FC1(F)CCC(Nc2nccc3[nH]nc(-c4ccccn4)c23)CC1] |
| 35 | N-isopropyl-3-(4-(1-methyl-1H-pyrazol-4- yl)pyridin-2-yl)-1H-pyrazolo[4,3-c]pyridin-4- amine | 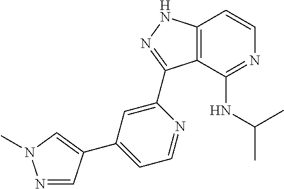 [SMILES: CC(C)Nc1nccc2[nH]nc(-c3cc(-c4cnn(C)c4)ccn3)c12] |
| 36 | N-isopropyl-3-(4-(1-methyl-1H-pyrazol-5- yl)pyridin-2-yl)-1H-pyrazolo[4,3-c]pyridin-4- amine | 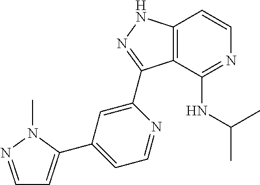 [SMILES: CC(C)Nc1nccc2[nH]nc(-c3cc(-c4ccnn4C)ccn3)c12] |
| 37 | 3-(2-fluoro-[3,4′-bipyridin]-2′-yl)-N-isopropyl- 1H-pyrazolo[4,3-c]pyridin-4-amine | 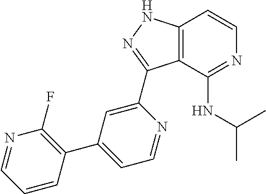 [SMILES: CC(C)Nc1nccc2[nH]nc(-c3cc(-c4cccnc4F)ccn3)c12] |
| 38 | N-(4,4-difluorocyclohexyl)-3-(pyrimidin-4-yl)- 1H-pyrazolo[4,3-c]pyridin-4-amine | 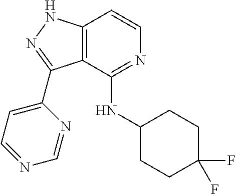 [SMILES: FC1(F)CCC(Nc2nccc3[nH]nc(-c4ccncn4)c23)CC1] |
| 39 | N-isopropyl-3-(4-(tetrahydro-2H-pyran-4- yl)pyridin-2-yl)-1H-pyrazolo[4,3-c]pyridin-4- amine | 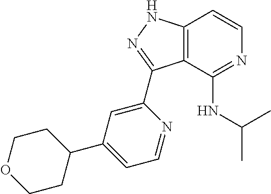 [SMILES: CC(C)Nc1nccc2[nH]nc(-c3cc(C4CCOCC4)ccn3)c12] |
| 40 | N-isopropyl-3-(4-(1-methylpiperidin-4- yl)pyridin-2-yl)-1H-pyrazolo[4,3-c]pyridin-4- amine | 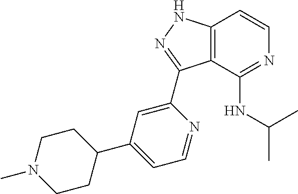 [SMILES: CC(C)Nc1nccc2[nH]nc(-c3cc(C4CCN(C)CC4)ccn3)c12] |
| 41 | 3-(5-cyclopropyl-4-methyl-4H-1,2,4-triazol-3- yl)-N-isopropyl-1H-pyrazolo[4,3-c]pyridin-4- amine | 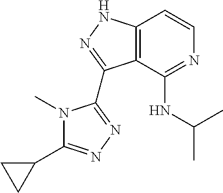 [SMILES: CC(C)Nc1nccc2[nH]nc(-c3nnc(C4CC4)n3C)c12] |
| 42 | 3-(5-cyclopropyl-1-methyl-1H-1,2,4-triazol-3- yl)-N-isopropyl-1H-pyrazolo[4,3-c]pyridin-4- amine | 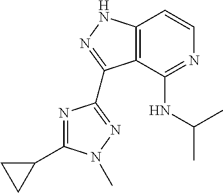 [SMILES: CC(C)Nc1nccc2[nH]nc(-c3nc(C4CC4)n(C)n3)c12] |
| 43 | 3-(pyridin-2-yl)-N-(2,2,2-trifluoroethyl)-1H- pyrazolo[4,3-c]pyridin-4-amine | 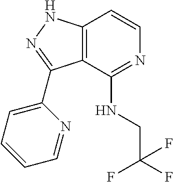 [SMILES: FC(F)(F)CNc1nccc2[nH]nc(-c3ccccn3)c12] |
| 44 | 3-(4-(1,5-dimethyl-1H-pyrazol-4-yl)pyridin-2- yl)-N-isopropyl-1H-pyrazolo[4,3-c]pyridin-4- amine | 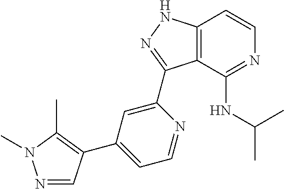 [SMILES: Cc1c(-c2ccnc(-c3n[nH]c4ccnc(NC(C)C)c34)c2)cnn1C] |
| 45 | 3-(4-(1,3-dimethyl-1H-pyrazol-4-yl)pyridin-2- yl)-N-isopropyl-1H-pyrazolo[4,3-c]pyridin-4- amine | 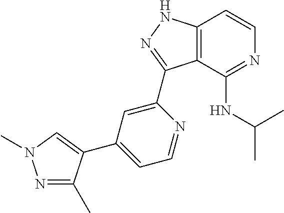 [SMILES: Cc1nn(C)cc1-c1ccnc(-c2n[nH]c3ccnc(NC(C)C)c23)c1] |
| 46 | N-isopropyl-3-(4-(1-methyl-1H-pyrazol-3- yl)pyridin-2-yl)-1H-pyrazolo[4,3-c]pyridin-4- amine | 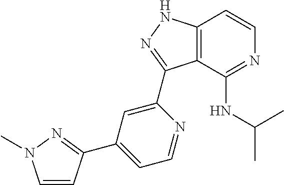 [SMILES: CC(C)Nc1nccc2[nH]nc(-c3cc(-c4ccn(C)n4)ccn3)c12] |
| 47 | N-isopropyl-3-(6-(tetrahydro-2H-pyran-4- yl)pyrimidin-4-yl)-1H-pyrazolo[4,3-c]pyridin-4- amine | 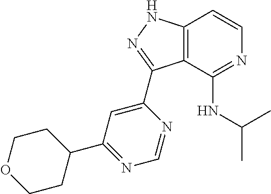 [SMILES: CC(C)Nc1nccc2[nH]nc(-c3cc(C4CCOCC4)ncn3)c12] |
| 48 | N-isopropyl-3-(6-(tetrahydrofuran-3- yl)pyrimidin-4-yl)-1H-pyrazolo[4,3-c]pyridin-4- amine | 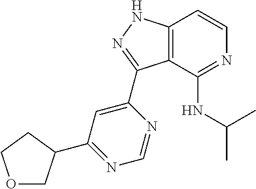 [SMILES: CC(C)Nc1nccc2[nH]nc(-c3cc(C4CCOC4)ncn3)c12] |
| 49 | N-isopropyl-3-(4-(tetrahydrofuran-3-yl)pyridin- 2-yl)-1H-pyrazolo[4,3-c]pyridin-4-amine | 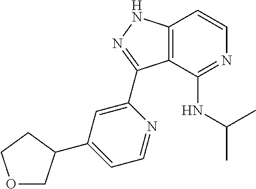 [SMILES: CC(C)Nc1nccc2[nH]nc(-c3cc(C4CCOC4)ccn3)c12] |
| 50 | 3-(4-ethoxypyridin-2-yl)-N-(tetrahydro-2H- pyran-4-yl)-1H-pyrazolo[4,3-c]pyridin-4-amine | 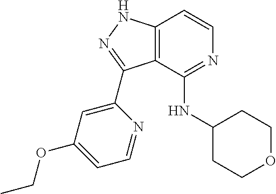 [SMILES: CCOc1ccnc(-c2n[nH]c3ccnc(NC4CCOCC4)c23)c1] |
| 51 | 6-methyl-N-(tetrahydro-2H-pyran-4-yl)-3-(4- (trifluoromethyl)pyridin-2-yl)-1H-pyrazolo[4,3- c]pyridin-4-amine | 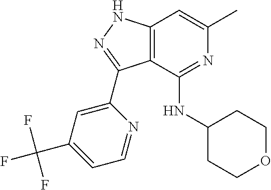 [SMILES: Cc1cc2[nH]nc(-c3cc(C(F)(F)F)ccn3)c2c(NC2CCOCC2)n1] |
| 52 | N,N-dimethyl-2-(4-((tetrahydro-2H-pyran-4- yl)amino)-1H-pyrazolo[4,3-c]pyridin-3- yl)isonicotinamide | 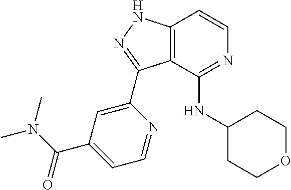 [SMILES: CN(C)C(=O)c1ccnc(-c2n[nH]c3ccnc(NC4CCOCC4)c23)c1] |
| 53 | N-isopropyl-3-(4-(tetrahydrofuran-2-yl)pyridin- 2-yl)-1H-pyrazolo[4,3-c]pyridin-4-amine | 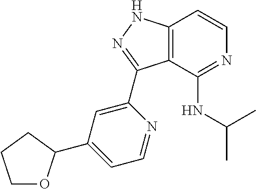 [SMILES: CC(C)Nc1nccc2[nH]nc(-c3cc(C4CCCO4)ccn3)c12] |
| 54 | N-isopropyl-3-(6-(tetrahydrofuran-2- yl)pyrimidin-4-yl)-1H-pyrazolo[4,3-c]pyridin-4- amine | 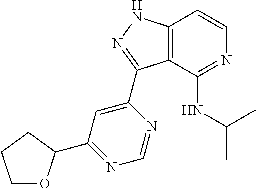 [SMILES: CC(C)Nc1nccc2[nH]nc(-c3cc(C4CCCO4)ncn3)c12] |
| 55 | 2-(4-(isopropylamino)-1H-pyrazolo[4,3- c]pyridin-3-yl)-N,N-dimethylisonicotinamide | 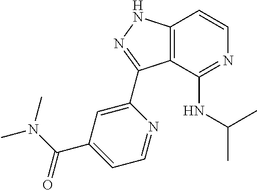 [SMILES: CC(C)Nc1nccc2[nH]nc(-c3cc(C(=O)N(C)C)ccn3)c12] |
| 56 | (2-(4-(isopropylamino)-1H-pyrazolo[4,3- c]pyridin-3-yl)pyridin-4- yl)(morpholino)methanone | 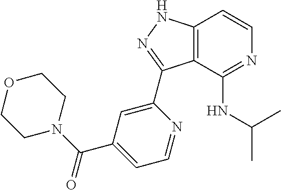 [SMILES: CC(C)Nc1nccc2[nH]nc(-c3cc(C(=O)N4CCOCC4)ccn3)c12] |
| 57 | 6-methyl-N-(tetrahydro-2H-pyran-4-yl)-3-(6- (trifluoromethyl)pyrimidin-4-yl)-1H- pyrazolo[4,3-c]pyridin-4-amine | 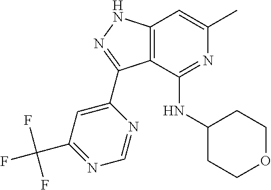 [SMILES: Cc1cc2[nH]nc(-c3cc(C(F)(F)F)ncn3)c2c(NC2CCOCC2)n1] |
| 58 | 3-(6-ethoxypyrimidin-4-yl)-6-methyl-N- (tetrahydro-2H-pyran-4-yl)-1H-pyrazolo[4,3- c]pyridin-4-amine | 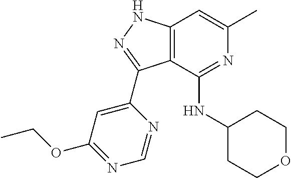 [SMILES: CCOc1cc(-c2n[nH]c3cc(C)nc(NC4CCOCC4)c23)ncn1] |
| 59 | 3-(4-((3R,4R)-3,4-difluoropyrrolidin-1- yl)pyridin-2-yl)-N-isopropyl-1H-pyrazolo[4,3- c]pyridin-4-amine | 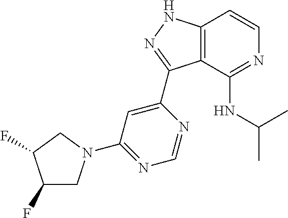 [SMILES: CC(C)Nc1nccc2[nH]nc(-c3cc(N4C[C@@H](F)[C@H](F)C4)ncn3)c12] |
| 60 | N,N-dimethyl-2-(6-methyl-4-((tetrahydro-2H- pyran-4-yl)amino)-1H-pyrazolo[4,3-c]pyridin-3- yl)isonicotinamide | 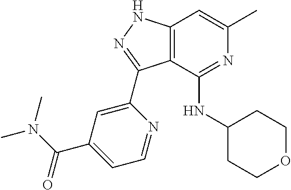 [SMILES: Cc1cc2[nH]nc(-c3cc(C(=O)N(C)C)ccn3)c2c(NC2CCOCC2)n1] |
| 61 | 3-(4-(2,2,6,6-tetrafluoromorpholino)pyridin-2- yl)-N-(tetrahydro-2H-pyran-4-yl)-1H- pyrazolo[4,3-c]pyridin-4-amine | 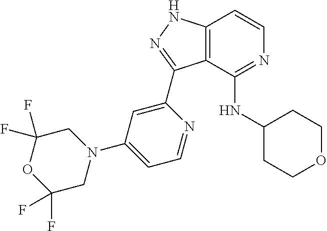 [SMILES: FC1(F)CN(c2ccnc(-c3n[nH]c4ccnc(NC5CCOCC5)c34)c2)CC(F)(F)O1] |
| 62 | 6-(4-(isopropylamino)-1H-pyrazolo[4,3- c]pyridin-3-yl)-N,N-dimethylpyrimidine-4- carboxamide | 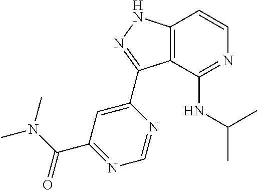 [SMILES: CC(C)Nc1nccc2[nH]nc(-c3cc(C(=O)N(C)C)ncn3)c12] |
| 63 | azetidin-1-yl(2-(4-((tetrahydro-2H-pyran-4- yl)amino)-1H-pyrazolo[4,3-c]pyridin-3- yl)pyridin-4-yl)methanone | 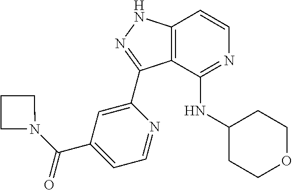 [SMILES: O=C(c1ccnc(-c2n[nH]c3ccnc(NC4CCOCC4)c23)c1)N1CCC1] |
| 64 | N,N,2-trimethyl-6-(4-((tetrahydro-2H-pyran-4- yl)amino)-1H-pyrazolo[4,3-c]pyridin-3- yl)isonicotinamide | 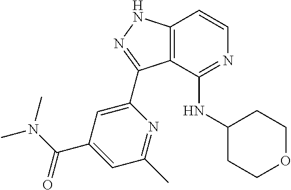 [SMILES: Cc1cc(C(=O)N(C)C)cc(-c2n[nH]c3ccnc(NC4CCOCC4)c23)n1] |
| 65 | N-(cyclopropylmethyl)-3-(6- (trifluoromethyl)pyrimidin-4-yl)-1H- pyrazolo[4,3-c]pyridin-4-amine | 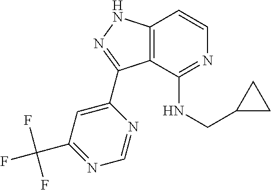 [SMILES: FC(F)(F)c1cc(-c2n[nH]c3ccnc(NCC4CC4)c23)ncn1] |
| 66 | N-(4,4-difluorocyclohexyl)-3-(6- (trifluoromethyl)pyrimidin-4-yl)-1H- pyrazolo[4,3-c]pyridin-4-amine | 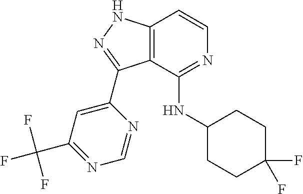 [SMILES: FC1(F)CCC(Nc2nccc3[nH]nc(-c4cc(C(F)(F)F)ncn4)c23)CC1] |
| 67 | N-(4,4-difluorocyclohexyl)-3-(2-methyl-6- (trifluoromethyl)pyrimidin-4-yl)-1H- pyrazolo[4,3-c]pyridin-4-amine | 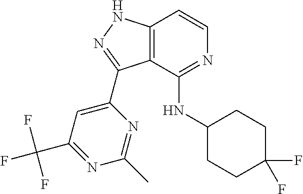 [SMILES: Cc1nc(-c2n[nH]c3ccnc(NC4CCC(F)(F)CC4)c23)cc(C(F)(F)F)n1] |
| 68 | N-(2,6-dimethyltetrahydro-2H-pyran-4-yl)-3-(6- (trifluoromethyl)pyrimidin-4-yl)-1H- pyrazolo[4,3-c]pyridin-4-amine | 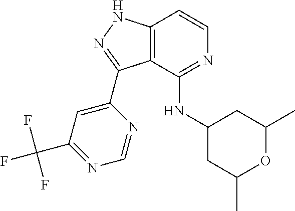 [SMILES: CC1CC(Nc2nccc3[nH]nc(-c4cc(C(F)(F)F)ncn4)c23)CC(C)O1] |
| 69 | 3-(4-(methylsulfonyl)pyridin-2-yl)-N- (tetrahydro-2H-pyran-4-yl)-1H-pyrazolo[4,3- c]pyridin-4-amine | 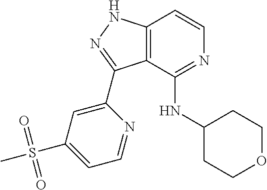 [SMILES: CS(=O)(=O)c1ccnc(-c2n[nH]c3ccnc(NC4CCOCC4)c23)c1] |
| 70 | 3-(6-methyl-4-(trifluoromethyl)pyridin-2-yl)-N- (tetrahydro-2H-pyran-4-yl)-1H-pyrazolo[4,3- c]pyridin-4-amine | 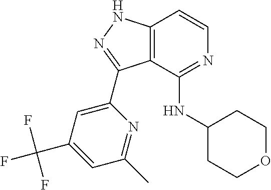 [SMILES: Cc1cc(C(F)(F)F)cc(-c2n[nH]c3ccnc(NC4CCOCC4)c23)n1] |
| 71 | 3-(2-methyl-6-(trifluoromethyl)pyrimidin-4-yl)- N-(tetrahydro-2H-pyran-4-yl)-1H-pyrazolo[4,3- c]pyridin-4-amine | 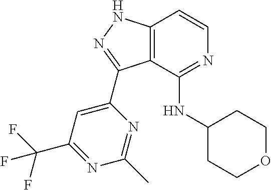 [SMILES: Cc1nc(-c2n[nH]c3ccnc(NC4CCOCC4)c23)cc(C(F)(F)F)n1] |
| 72 | N-isopropyl-3-(4-(methylsulfonyl)pyridin-2-yl)- 1H-pyrazolo[4,3-c]pyridin-4-amine | 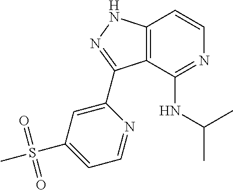 [SMILES: CC(C)Nc1nccc2[nH]nc(-c3cc(S(C)(=O)=O)ccn3)c12] |
| 73 | N-(dicyclopropylmethyl)-3-(6- (trifluoromethyl)pyrimidin-4-yl)-1H- pyrazolo[4,3-c]pyridin-4-amine | 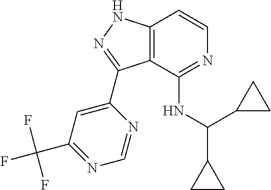 [SMILES: FC(F)(F)c1cc(-c2n[nH]c3ccnc(NC(C4CC4)C4CC4)c23)ncn1] |
| 74 | N-isopropyl-3-(4-(isopropylsulfonyl)pyridin-2- yl)-1H-pyrazolo[4,3-c]pyridin-4-amine | 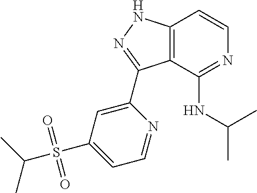 [SMILES: CC(C)Nc1nccc2[nH]nc(-c3cc(S(=O)(=O)C(C)C)ccn3)c12] |
| 75 | 3-(4-(isopropylsulfonyl)pyridin-2-yl)-N- (tetrahydro-2H-pyran-4-yl)-1H-pyrazolo[4,3- c]pyridin-4-amine | 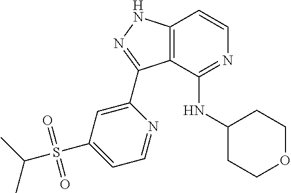 [SMILES: CC(C)S(=O)(=O)c1ccnc(-c2n[nH]c3ccnc(NC4CCOCC4)c23)c1] |
| 76 | N-isopropyl-3-(6-methyl-4- (methylsulfonyl)pyridin-2-yl)-1H-pyrazolo[4,3- c]pyridin-4-amine | 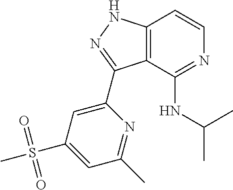 [SMILES: Cc1cc(S(C)(=O)=O)cc(-c2n[nH]c3ccnc(NC(C)C)c23)n1] |
| 77 | N-(8-oxabicyclo[3.2.1]octan-3-yl)-3-(6- (trifluoromethyl)pyrimidin-4-yl)-1H- pyrazolo[4,3-c]pyridin-4-amine | 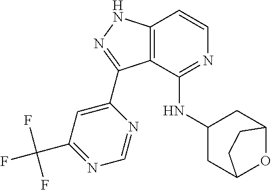 [SMILES: FC(F)(F)c1cc(-c2n[nH]c3ccnc(NC4CC5CCC(C4)O5)c23)ncn1] |

and pharmaceutically acceptable salts thereof.

17. A pharmaceutical composition comprising a compound according to claim 1 and a pharmaceutically acceptable carrier, diluent or excipient.

18. A method of treating a mammal having a disease state alleviated by the inhibition of LRRK2, wherein the method comprises administering to a mammal a therapeutically effective amount of a compound according to claim 1.

19. A process for preparing a compound of formula (1A) or (1B) as defined in claim 1, said process comprising the steps of:
(i) converting a compound of formula (4) into a compound of formula (3), where PG is a protecting group;
(ii) reacting said compound of formula (3) with a compound of formula R¹—NH₂ to form a compound of formula (2);
(iii) converting said compound of formula (2) to a compound of formula (1A) or (1B)

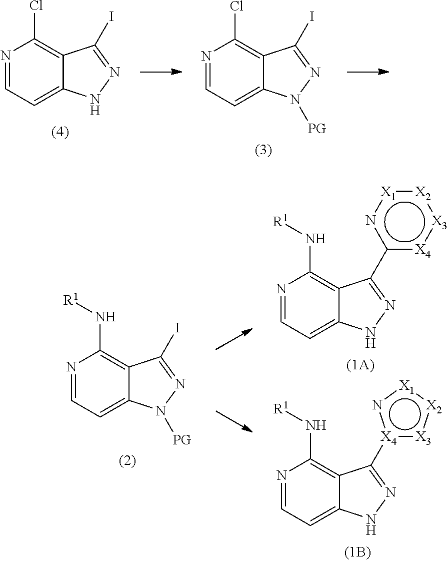

20. A process according to claim 24 wherein step (iii) comprises reacting said compound of formula (2) with 1,1,1,2,2,2-hexamethyldistannane and trans-Pd(PPh₃)₂Cl₂ to form an intermediate of formula (2A): 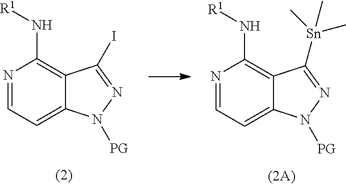
and converting said compound of formula (2A) into a compound of formula (1A) or (1B).
